(19) United States
(12) Patent Application Publication
(10) Pub. No.: US 2024/0344004 A1
(43) Pub. Date: Oct. 17, 2024

(54) HUMAN-ON-CHIP OPERATING SYSTEM

(71) Applicant: DLOC BIOSYSTEMS, INC., Middletown, DE (US)

(72) Inventors: Waddah MALAEB, London (GB); Giulia GRIMALDI, Oslo (NO); Mustafa AMMOURI, Kaifoun (LB); Farid MALAEB, Baissour (LB); Bahaa Eddine EL ARIDI, Baissour (LB)

(21) Appl. No.: 18/751,005

(22) Filed: Jun. 21, 2024

Related U.S. Application Data
(63) Continuation-in-part of application No. PCT/US2022/054004, filed on Dec. 23, 2022.
(60) Provisional application No. 63/293,422, filed on Dec. 23, 2021.

(51) Int. Cl. C12M 3/00; B01L 3/00; C12M 1/36; C12M 3/06

(52) U.S. Cl. CPC C12M 21/08; B01L 3/502715; C12M 23/16; C12M 41/48; B01L 2200/10; B01L 2300/0819; B01L 2400/0694

(57) ABSTRACT

A human-on-chip plate. The human-on-chip plate may include, at least one 3D culture biochip, at least one micro-valve, at least one microfluidic channel, at least one inlet or outlet plate port. The outlet ports from one chip may be connected to another chip by routing the valves. Multiple chips may be connected in parallel, in series or in combination and one or more chips in the human-on-chip plate may be bypassed. Each fluid connection may be altered independently and at any time. The at least one valving system connects or separates different compartments of and between the organ-on-chip system. The at least one inlet and one outlet ports to access and block the microchannels. The at least one valving system could allow for sampling, changing the model flow map, and introduce or reduce at least one fluidic chamber.

### CROSS-REFERENCE TO RELATED APPLICATIONS

This application is a continuation-in-part of PCT/US2022/054004, entitled “HUMAN-ON-CHIP OPERATING SYSTEM”, filed Dec. 23, 2022, which claims priority to and the benefit of U.S. Provisional Patent Application No. 63/293,422 filed Dec. 23, 2021, the entire contents of which are incorporated by reference herein.

### BACKGROUND

Developing a successful pharmaceutical drug is expensive and time-consuming. Additionally, many drugs fail in the late stages of the development process, causing significant sunk costs. One contributing reason for drugs failing in late developmental stages is that pharmaceutical companies typically test drugs on cells grown on cell culture platforms that do not sufficiently well replicate human tissue. The 3D organ-on-chip (OOC) technology emerged to create better resembling tissue models solving the structural and 3D tissue assembly problem. However, there persists the problem of operating systems in a way that replicates the mechanics, and the fluid dynamics of operating system advanced 3D models while feeding, maintaining, and growing the tissues, and real-time measuring & analyzing the aimed parameters in operating system advanced systems.

Current cell culture experiments often do not accurately mimic physiological conditions as they use simplistic approaches, which do not reproduce the entire system, but rather only a few aspects. Multiple factors go hand-in-hand in developing & operating 3D advanced cell culture models. Operating system factors span a wide range of design parameters, including the level of complexity of the model, the connection mechanism between the different organ models, the pumping, seeding, and sampling mechanisms, and also the measurement, sensing & imaging mechanisms, followed by the analysis and prediction techniques. A holistic system spanning all later factors is needed to get precise drug prediction from preclinical testing.

One main enduring issue facing the current tissue culture semi-automated operating systems is the need for relatively large volumes, tens of milliliters, of materials that are wasted just to seed and use only a few microliters of cells and biological materials. This limits the culturing and modeling of tissues using expensive or scarce materials such as biopsies.

Another aspect of the operation of a complex 3D culture model is the analytical read-outs which limit high-throughput analysis as well as real-time data collection. Furthermore, often accessing cell and/or media samples for analysis at different time points is challenging in the current organ-on-chips. Media is defined as any fluid containing any of the following: therapeutic agents, growth factors, nutrients, chemicals, cells in suspension, and/or any other molecule, biomolecule, or substance flowing through the channels.

Typical complex 3D cell culture platforms are incapable of real-time imaging or measuring biological parameters of separate tissues connected into a human-on-chip (HOC) model while operating and flowing media into the biochip. One of the limitations is in the size of the design that prevents the placement of one or more analytical devices in proximity to the biochips. For example, to model the metastasis of cancer from one tissue to another through 3D vasculature, one cannot image both tissues simultaneously while pumping the fluids through the blood vessel. Similarly, much of the valuable data generated is, in most cases, restricted to a few data points rather than continuous measurements.

One additional aspect of the typical biochip designs, and the connectivity method used between the organs, is the inability to set up complex experiments having multi-organs while preserving accurate sensing and communication. These systems lack the communication and sensing that is found in the human body, as well as the ability to set up any experiment by a biochip pattern.

One main limitation these systems face while sampling and inserting the media is the need for high volumes of fluids to compensate for the volume of the tubes in the system. The usage of ultra-thin tubes or micro-channels prevents the need for a dilution factor, which is used in other systems to move the fluids. However, such low-diameter tubes maintain high capillary forces and a higher surface tension inside the tubes, wherein a low fluid volume can be transferred to a further destination. Also, not having a dilution factor ensures a highly concentrated sample with the molecules that need to be detect, thus, more accurate measurements and results.

One important aspect of a cell culture experiment is the ability to measure and read physiologically relevant data accurately. Microscopy, spectroscopy, and Fourier Transform Infrared Spectroscopy (FTIR) are some of the widely used techniques to perform quantitative measurements on biological materials, which indicate cell viability, functionality, and metabolism. Most human-on-chip operating systems currently found in the market depend on external measuring devices to perform such assays. These tests will require the operator to halt the experiment and transfer it to the measuring device. This causes missing valuable observations and data in between the set time points. Performing such manipulation also increases the number of variables, which may cause an unplanned effect on the experiment results. There is a dire need for implementing multiplexed real-time measurements on running cell-culture experiments.

### SUMMARY

The present disclosure provides a new and innovative biochip operating system that acts as a cell culture platform that models and operates on multiple human organs simultaneously. The operating system contains and operates on a human-on-chip plate, which is an array of multiple biochips in fluid communication via ducts modeling multiple organs of the human body. Cells can be inserted into the biochip and grow into 3-dimensional tissues to be used in drug testing. Each biochip may contain one or multiple ultra-thin porous plastic cylindrical-shaped ducts. Each duct can be accessed from the inside or from the area surrounding the ducts through microfluidic channels.

The present disclosure provides, due to the biochips, a means to replicate a ductal organoid microenvironment by growing the ductal cells on the inner walls of each duct and growing the surrounding tissue from the outside by seeding cells and delivering media components through a gel from the other side surrounding the ducts. Each biochip may be utilized to replicate ductal or non-ductal tissues including, but not limited to: pancreatic, renal, hepatic, breast, brain, lung, vasculature, prostate, fallopian tube, testicular, and lymphatic. The human-on-chip plate is an array of biochips, each growing and modeling a specific tissue and in fluid communication, either directly or indirectly, with the other biochips through one or more ducts of each biochip. The biochip ducts may be separated by a valving system that allows for the connection between different biochips. These biochips can be connected in series, in parallel, or in any combination of those. Furthermore, the valving system allows for these connections to be altered at any point. The biochips may also be connected to a sampling chamber, the ATR-FTIR, or a waste chamber.

The present disclosure provides a biochip operating system comprising a semi- or fully-automated device that operates on the human-on-chip plate to pump and control the flow of fluids containing cell growth media, chemicals, and cells into each of the biochip's micro-channels. The biochip operating system may accomplish this without diluting the sample. The human operating system (HOS) also contains sensing and measurement devices to measure and control different biological parameters of the tissues grown inside the biochips. The fluidic valves and pumps actuation and the sensing and imaging outputs, are fed into a computer software that automates the cell culture and measurement process. The human operating system could be used by, but is not limited to, researchers, pharmaceutical companies, and clinical physicians to test their drugs or other components on full human models made of cells from biopsies, primary cells, stem cells, and/or cell lines. This system may be configured to perform, control, and measure full cell culture experiments of a wide range of applications.

The present disclosure provides a system containing devices and methods to pump and control the flow of very small volumes (as low as 0.1 μl) of fluids containing cells, media, therapeutic agents, chemicals, reagents, and other biological components into each channel inside every individual biochip. The disclosure allows operating precise, repeatable, and reproducible biological experiments on tissue models of very high resemblance.

The present disclosure also provides implementing multiplexed real-time measurements on running cell-culture experiments. For instance, implementing an onboard triple measuring system consisting of microscopy, spectroscopy, and Attenuated Total Reflectance sampling for FTIR (ATR-FTIR) with tight integration with the operating system will provide more valuable information and a better representation of the experiment while eliminating any variables that might form due to the culture relocation. The importance of having multiple measurement techniques performing various readings simultaneously while being connected to the same data reader is that it allows for obtaining a multiplexed analysis and conclusions impossible otherwise.

In another aspect of the present disclosure, which may be used in combination with any other aspect or combination of aspects listed herein, a biochip operating system includes at least one biological system on the biochip plate, at least one valving means, at least one actuation means for actuating the valving means, at least one fluidic handling means, at least one analytical device, and; a control system for controlling the pumping means, the actuation means, and valving means, and the analytical device.

In another aspect of the present disclosure, which may be used in combination with any other aspect or combination of aspects listed herein, the analytical device may inspect biochips utilizing at least one of the microscope images or spectral measurements.

In another aspect of the present disclosure, which may be used in combination with any other aspect or combination of aspects listed herein, the analytical device may inspect media in the fluidic handling means or fluidic pathways.

In another aspect of the present disclosure, which may be used in combination with any other aspect or combination of aspects listed herein, the operating system facilitates, semi-automates, or automates at least one aspect of the experimental setup, including, but not limited to, at least one of the cleaning, sterilizing or preparing the system; priming the system with a biocompatible fluid; populating the biochips with the correct cell types in the correct locations as required for the desired experiment; selectively connecting the biochips in the correct arrangement for the desired experiment; and/or metering the correct amount of media or other compounds into the system.

In another aspect of the present disclosure, which may be used in combination with any other aspect or combination of aspects listed herein, the operating system facilitates, semi-automates, or automates at least one aspect of experimental execution, including, but not limited to, at least one of circulating the media between biochips as required for the experiment; controlling flow rates of fluidic movements; extracting samples, possibly at specific times or time intervals; inspecting using analytical means at specific times or time intervals and/or isolating and fixing biochips for later analysis. Samples may be extracted on-the-spot.

In another aspect of the present disclosure, which may be used in combination with any other aspect or combination of aspects listed herein, the operating system further comprises a motion stage to move the biological system on the biochip plate relative to the analytical device and/or actuation means.

In another aspect of the present disclosure, which may be used in combination with any other aspect or combination of aspects listed herein, a biochip for growing multi-ductal tissue includes a chassis, a plurality of internal regions, wherein the chassis contains the plurality of the internal regions, wherein at least one of the internal regions is in permeable or semipermeable communication with at least one other internal region and a plurality of fluidic media.

In another aspect of the present disclosure, which may be used in combination with any other aspect or combination of aspects listed herein, a plurality of internal regions comprise tubular ducts.

In another aspect of the present disclosure, which may be used in combination with any other aspect or combination of aspects listed herein, at least two tubular ducts are arranged with an inlet and outlet to permit a through-flow of the first fluidic medium.

In another aspect of the present disclosure, which may be used in combination with any other aspect or combination of aspects listed herein, at least two tubular ducts are arranged with an inlet and outlet distinct from those of a first duct to permit a through-flow of the second fluidic medium that may be distinct from the first fluidic medium.

In another aspect of the present disclosure, which may be used in combination with any other aspect or combination of aspects listed herein, at least one region comprises a stromal compartment disposed within the biochip chassis and further disposed externally to the at least two tubular ducts.

In another aspect of the present disclosure, which may be used in combination with any other aspect or combination of aspects listed herein, the plurality of fluidic media are the same media.

In another aspect of the present disclosure, which may be used in combination with any other aspect or combination of aspects listed herein, the plurality of fluidic media are different media.

In another aspect of the present disclosure, which may be used in combination with any other aspect or combination of aspects listed herein, the plurality of fluidic media is a combination of fluidic media types.

In another aspect of the present disclosure, which may be used in combination with any other aspect or combination of aspects listed herein, a biochip fluidic control includes at least one human-on-chip containing at least one organ-on-chip system, wherein the at least one organ-on-chip system containing at least one ductal scaffold interfacing at least one surrounding compartment, at least one valving system controlling the flow to each compartment, at least one micro-pumping mechanism that can pump and control the flow to at least one compartment, at least one actuator controlling the valves on the biochip and leading to the biochip, at least one actuator controlling the fluid pumping to the biochip compartments, wherein the at least one valving system could open or close and shift the flow pathways to every compartment.

In another aspect of the present disclosure, which may be used in combination with any other aspect or combination of aspects listed herein, the at least one valving mechanism may be at least one of hydraulically, pneumatically, mechanically, electrically, and fluidically actuated.

In another aspect of the present disclosure, which may be used in combination with any other aspect or combination of aspects listed herein, the at least one valving mechanism connects the at least one biochip to the operating system.

In another aspect of the present disclosure, which may be used in combination with any other aspect or combination of aspects listed herein, the at least one valving mechanism connects the at least two biochips in a human-on-chip connection plate.

In another aspect of the present disclosure, which may be used in combination with any other aspect or combination of aspects listed herein, the at least one valving mechanism controls the at least two biochips in the human-on-chip connection plate.

In another aspect of the present disclosure, which may be used in combination with any other aspect or combination of aspects listed herein, the at least one valving mechanism controls the at least one inlet and outlet of the biochip.

In another aspect of the present disclosure, which may be used in combination with any other aspect or combination of aspects listed herein, the at least one valving mechanism connects and controls the connection between the at least one duct to another duct.

In another aspect of the present disclosure, which may be used in combination with any other aspect or combination of aspects listed herein, the at least one valving mechanism connects the at least one stroma to another stroma.

In another aspect of the present disclosure, which may be used in combination with any other aspect or combination of aspects listed herein, the at least one valving mechanism connects and controls the connection between at least one duct to at least one stroma.

In another aspect of the present disclosure, which may be used in combination with any other aspect or combination of aspects listed herein, the at least one valving mechanism connects the at least one biochip to at least one measuring device.

In another aspect of the present disclosure, which may be used in combination with any other aspect or combination of aspects listed herein, the at least one valving mechanism contains at least one bistable valve.

In another aspect of the present disclosure, which may be used in combination with any other aspect or combination of aspects listed herein, the at least one valving mechanism contains at least one open-close valve.

In another aspect of the present disclosure, which may be used in combination with any other aspect or combination of aspects listed herein, the at least one valving mechanism contains at least one bistable and open-close valves.

In another aspect of the present disclosure, which may be used in combination with any other aspect or combination of aspects listed herein, the at least one bistable valve maintains a state position by a beam, spring, magnet, or any drilled geometrical shape, or the usage of smart materials such as memory shape alloy.

In another aspect of the present disclosure, which may be used in combination with any other aspect or combination of aspects listed herein, the at least one open close valve maintains a state position by a drilled geometrical shape.

In another aspect of the present disclosure, which may be used in combination with any other aspect or combination of aspects listed herein, the at least one valving mechanism controls the media insertion in at least one biochip.

In another aspect of the present disclosure, which may be used in combination with any other aspect or combination of aspects listed herein, the at least one valving mechanism controls the cell insertion in at least one biochip.

In another aspect of the present disclosure, which may be used in combination with any other aspect or combination of aspects listed herein, the at least one valving mechanism maintains a cell culture by media insertion.

In another aspect of the present disclosure, which may be used in combination with any other aspect or combination of aspects listed herein, a human-on-chip plate including at least one 3D culture biochip, at least one micro-valve, at least one micro-fluidic channel, at least one inlet or outlet plate port, wherein the outlet ports from one biochip may be connected to another biochip by routing the valves, wherein multiple biochips may be connected in parallel, in series or in any combination of those and one or more biochips in the human-on-chip plate can be bypassed, wherein each fluid connection may be altered independently and at any time, wherein the at least one valving system connecting or separating different compartments of and between the organ-on-chip system, wherein at least one inlet and one outlet ports to access and block the micro-channels, wherein the at least one valving system could allow for sampling, changing the model flow map, and introduce or reduce at least one fluidic chamber, wherein the valves, which may be controlled in a bistable position by the actuators and the sensors, all positioned at the channel ports of the biochips wherein, the valves are positioned as immediately adjacent to the biochips and may be controlled in a bistable position.

In another aspect of the present disclosure, which may be used in combination with any other aspect or combination of aspects listed herein, the human-on-chip plate includes at least one organ-on-chip system including at least one ductal scaffold interfacing at least one surrounding compartment, wherein, at least one organ-on-chip system containing at least one ductal scaffold interfacing at least one surrounding compartment.

In another aspect of the present disclosure, which may be used in combination with any other aspect or combination of aspects listed herein, the human-on-chip plate may be rapidly connected from a side, bottom, or upper side, wherein fluids will be exchanged.

In another aspect of the present disclosure, which may be used in combination with any other aspect or combination of aspects listed herein, the human-on-chip plate is interchangeable, wherein ports on the human-on-chip will align with fittings in the biochip, while the valves are fixed or removable.

In another aspect of the present disclosure, which may be used in combination with any other aspect or combination of aspects listed herein, the human-on-chip plate includes interconnected built-in biochips.

In another aspect of the present disclosure, which may be used in combination with any other aspect or combination of aspects listed herein, the valves are fixed or removable.

In another aspect of the present disclosure, which may be used in combination with any other aspect or combination of aspects listed herein, the human-on-chip allows for the fluid to be accessed from at least one of a top, bottom, or the side.

In another aspect of the present disclosure, which may be used in combination with any other aspect or combination of aspects listed herein, a multiplex logic micro-valve system includes a plurality of micro-valves, at least one fluidic pathway including at least one channel, an inlet and outlet port, at least one bistable mechanism, at least one actuating mechanism, at least one structure which encloses the other elements of the micro-valve, wherein the fluid pathway has no dead volumes, wherein multiplexing between the plurality of valves reduces the amount of actuating inputs and wherein the valves may control the fluid within a channel so that the flow can be either hydrostatic or in motion.

In another aspect of the present disclosure, which may be used in combination with any other aspect or combination of aspects listed herein, at least one logic multiplex micro-valve is used to control fluid in the systems.

In another aspect of the present disclosure, which may be used in combination with any other aspect or combination of aspects listed herein, the valves may direct a sample to at least one sampling port.

In another aspect of the present disclosure, which may be used in combination with any other aspect or combination of aspects listed herein, the bistable mechanism may be a bistable beam on a flexible tube valve.

In another aspect of the present disclosure, which may be used in combination with any other aspect or combination of aspects listed herein, the fluidic pathway may connect at least one biochip to a plurality of components within a human-on-chip system.

In another aspect of the present disclosure, which may be used in combination with any other aspect or combination of aspects listed herein, the bistable valves have at least two stable positions.

In another aspect of the present disclosure, which may be used in combination with any other aspect or combination of aspects listed herein, at least one channel contains a balloon that is at least one of hydraulically, pneumatically, mechanically, electrically, and fluidically actuated.

In another aspect of the present disclosure, which may be used in combination with any other aspect or combination of aspects listed herein, the system may only require power when switching between states, and once actuated, the bistable beam will remain in position.

In another aspect of the present disclosure, which may be used in combination with any other aspect or combination of aspects listed herein, the micro-valve may be a bistable magnetic drilled-piston valve.

In another aspect of the present disclosure, which may be used in combination with any other aspect or combination of aspects listed herein, the bistable mechanism may contain magnetic channels.

In another aspect of the present disclosure, which may be used in combination with any other aspect or combination of aspects listed herein, the bistable beam of the micro-valve may be at least one of solenoidally, hydraulically, pneumatically, mechanically, electrically, and fluidically actuated.

In another aspect of the present disclosure, which may be used in combination with any other aspect or combination of aspects listed herein, a hybrid human-on-chip tissue culture platform for a human model is provided, wherein the hybrid human-on-chip tissue culture platform can be used by itself and/or with the aid of an operating system. The platform may be configured to control multiple OOCs, which may be isolated or connected to each other in customizable permutations that may be changed during an experiment. The platform may supply each of the connected OOCs with similar or different fluid components, flow rates, pressures, or shear forces.

In another aspect of the present disclosure, which may be used in combination with any other aspect or combination of aspects listed herein, a manufacturing and assembly process of a 2-duct, 1-stroma biochip, with three parallel channels is provided, wherein a side of a membrane is tightly bonded to a lower chassis, and a rod is placed inside a ductal area over the membrane allowing the membrane to rotate around the rod in a cylindrical manner of at least 180° orientation.

In another aspect of the present disclosure, which may be used in combination with any other aspect or combination of aspects listed herein, a 2-duct, 1-stroma biochip with three parallel channels is provided. The biochip may comprise a lower and upper chassis, two porous cylindrical ductal scaffolds channels that are surrounded by one stroma and covered with a bottom glass cover and an upper glass cover. Two ducts with porous membranes may be surrounded by one stroma, in which upper middle ducts are connected to a stroma chamber, wherein these two holes can be used for stroma feeding. In an additional embodiment, two types of epithelial or endothelial cells may be cultured in the ducts, and at least one of stromal, endothelial, or cancerous cells may be cultured in a stroma.

In another aspect of the present disclosure, which may be used in combination with any other aspect or combination of aspects listed herein, the biochip may include two porous cylindrical ductal scaffolds channels surrounded by a stroma chamber having a stroma feeding channel, wherein fluids insertion into the stroma may be performed by a perforated tube that enables direct feeding to the stroma, and wide pores in the tube may increase the diffusion rate between the perforated tube and the stroma.

In another aspect of the present disclosure, which may be used in combination with any other aspect or combination of aspects listed herein, a stroma chamber further comprises side pins and may be covered by a bottom glass cover and an upper glass cover.

In another aspect of the present disclosure, which may be used in combination with any other aspect or combination of aspects listed herein, the individual biochip entities are incorporated as an integrated human-on-chip. In a further aspect, a cylindrical rotational mechanism valve may connect several integrated biochips, wherein an integrated biochip comprises a membrane sandwiched in between top and bottom plates, wherein the membrane may include at least one porous cylindrical ductal scaffold channel surrounded by at least one stroma chamber, having a stroma feeding channel inlet and outlet, and air filtering areas, and a stroma area covered using an upper and lower glass cover. In another aspect, fluid is pumped through the side ducts of the human-on-chip plate and from one duct to another through a valving system.

In another aspect of the present disclosure, which may be used in combination with any other aspect or combination of aspects listed herein, biochip entities are incorporated as one human-on-chip plate that comprises several individual biochips where each individual biochip consists of two identical sections, inlet section and outlet section, and each part comprises a first duct, one inlet/outlet hole and a separation valve, and a second duct, a second inlet/outlet hole and a second separation valve, wherein these entities connect the biochips to each other. In a further aspect, the biochip inlet section comprises a duct one valves set and duct two valves set, wherein each set comprises three pneumatically actuated valves, where a main valve controls the sample taken from the duct inlet, and if opened, two valves control the flow of the sample towards the waste, or to the duct one inlet to operating system and duct two inlet to operating system. In a further aspect, the biochip comprises a stroma inlet/outlet and a controlling valve, and a pneumatically actuated valve controls the sample taken from the stroma inlet, and if opened, the flow of the sample is controlled by two pneumatically actuated valves, a waste valve that takes the sample to the waste, and a valve to the stroma inlet to operating system.

In another aspect of the present disclosure, which may be used in combination with any other aspect or combination of aspects listed herein, individual biochip entities are incorporated as one human-on-chip plate where a cylindrical rotational mechanism connects a chassis and a valving mechanism to connect individual biochip components, and fluid is pumped through a duct from the top of the human-on-chip plate and from one duct to another through a valving system.

In another aspect of the present disclosure, which may be used in combination with any other aspect or combination of aspects listed herein, a multiplex logic micro-valve system can actuate separate groups of valves, in a logic-coded format. In a further aspect, in-plate valves that connect channels of adjacent chips within a HOC plate can be micro-channeled and multiplexed together. In a further aspect, another system can run in parallel to multiplex the valves in the HOC that pump fluid from micro media chambers to individual chips in the HOC.

In another aspect of the present disclosure, which may be used in combination with any other aspect or combination of aspects listed herein, in-plate valves that connect the channels of adjacent chips within a HOC plate are actuated through a multiplex logic micro-valve system. In a further aspect, multiplexing of microvalves may be based on multi-signal microvalves that require multiple separate signals to open, and a matrix of microchannels passing signals to the microvalves.

In another aspect of the present disclosure, which may be used in combination with any other aspect or combination of aspects listed herein, in a multiplex logic micro-valve system, a bistable beam one is indicates the position of the valve on a specific row. In another aspect, in all the valves in row one, the first beam is connected through the plate channels to one signal indicating row one. In another aspect, another signal, row two, goes to the first beam of the valves in row two. In another aspect, a third signal, row three, connects the first beam of valves in row three. In another aspect, the second beam and third beam of the valves in this embodiment are connected to the channels along columns one or two and three or four respectively. In another aspect, beam four may be connected to valves channeled to either of the biochip micro-channels types, duct one, duct two, or stroma feeding channel.

In another aspect of the present disclosure, which may be used in combination with any other aspect or combination of aspects listed herein, in a multiplex logic micro-valve system, beams where no logical signal is acted on them are all signaled to open together in one signal after initiating valves by normally closing them using a common bottom balloon channel signal.

In another aspect of the present disclosure, which may be used in combination with any other aspect or combination of aspects listed herein, in a multiplex logic micro-valve system, all of a plurality of signals work together in a logic sequence, such that to selectively open only the valve of row one, column one, duct one, a signal coded (1, 1, P, 1) is initiated so that all beams on a valve are open and a flow can be accessed through this valve. In a further aspect, the code indicates the beams position on each valve (beam one, beam two, beam three, beam four), and the signal indicates the signal acted on the coded valves for each beam. In another aspect, those four signals can trigger and open a selected valve through the multiplex channeling through a code of (Row number, Column one or two number, Column three or four number, Chip channel number). In another aspect, row number could be one, two, or three acting on beam one. In another aspect, beam two column number could be one, two, or “P” acting on beam two. In another aspect, beam three column number could be three, four, or “P” acting on beam three. In another aspect, chip channel number could be one, two, or three acting on beam four. In another aspect, the row and column number indicates the position of the valve on the plate in a 2D view, the biochip channel number indicates which valve at this location is to be triggered (duct 1, duct 2, or stroma), and “P” indicates the valves that are initially opened at the initiation of the valves after normally closing them in the bottom balloon valves signal.

In another aspect of the present disclosure, which may be used in combination with any other aspect or combination of aspects listed herein, in a multiplex logic micro-valve system, the logic system is made to reduce the number of signals to actuate as many valves as possible allowing for fitting and controlling a large number of valves with a minimum number of actuators.

In another aspect of the present disclosure, which may be used in combination with any other aspect or combination of aspects listed herein, a heating plate contains an integrated heating element for thermal control, in order to control, increase, decrease, or maintain, a temperature of a cell culture.

In another aspect of the present disclosure, which may be used in combination with any other aspect or combination of aspects listed herein, a media may be stored in a mixing chamber. In another aspect, an air trap placed directly after the mixing chamber is configured to reduce the risk of bubbles formation blocking a flow. In another aspect, a set of 2/2 bistable pneumatically actuated valves are configured to control the flow from the mixing chamber to a media chamber. In another aspect, a fluid may pass through a pneumatically actuated piston that may be configured to extend and retract to pump the fluid. In another aspect, the 2/2 bistable pneumatically actuated valves are configured to actuate between an open/close position to prevent backflow of the fluid. In a further aspect, a two-duct biochip may be connected to a one-duct biochip through a 3/3 spring-loaded pneumatically actuated valve. In another aspect, the 3/3 spring-loaded pneumatically actuated valves connect all different aspects of a human operating system, and the media will be pumped by the pneumatically actuated piston from the media chamber through the 3/3 spring-loaded pneumatically actuated valves that actuated in the open position into a biochip. In another aspect, a sample may be extracted from the biochip into the media chamber and travel through multiple 2/2 bistable pneumatically actuated valves and 3/3 spring-loaded pneumatically actuated valves into different biochips found on a human-on-chip plate.

In another aspect of the present disclosure, which may be used in combination with any other aspect or combination of aspects listed herein, pressure sensors at each of a plurality of biochip inlet and outlet fluid ports are provided and may be configured to manipulate a pressure inside a plurality of channels of a plurality of biochips. In another aspect, using a pressure sensor and 2/2 bistable pneumatically actuated valve that may be situated at the inlet and outlet of the first duct, stoma, and second duct, a fluid is controllably perfused from the first duct or the second duct to the stoma or vice versa.

In another aspect of the present disclosure, which may be used in combination with any other aspect or combination of aspects listed herein, a mechanical micro-valve is provided.

In another aspect of the present disclosure, which may be used in combination with any other aspect or combination of aspects listed herein, a mechanical micro-valve connects two adjacent biochips together by connecting the ducts outlets on a first biochip to next biochip inlets through inlet/outlet connectors.

In another aspect of the present disclosure, which may be used in combination with any other aspect or combination of aspects listed herein, a mechanical micro-valve allows exchange of any fluid passing through, or lets the fluid pass through without interventions.

In another aspect of the present disclosure, which may be used in combination with any other aspect or combination of aspects listed herein, a mechanical micro-valve has at least two states, the states switch between a valve allowing or blocking fluid flow passing through lower channels.

In another aspect of the present disclosure, which may be used in combination with any other aspect or combination of aspects listed herein, two interlinked pistons situated inside valve compartments move in opposite directions simultaneously controlling a state of the valve.

In another aspect of the present disclosure, which may be used in combination with any other aspect or combination of aspects listed herein, pistons may be hydraulically actuated.

In another aspect of the present disclosure, which may be used in combination with any other aspect or combination of aspects listed herein, a mechanical micro-valve connects two biochips through microfluidic channels.

In another aspect of the present disclosure, which may be used in combination with any other aspect or combination of aspects listed herein, a mechanical micro-valve contains two drilled rotating cylinders.

In another aspect of the present disclosure, which may be used in combination with any other aspect or combination of aspects listed herein, each cylinder has three channels, two channels located on a radial direction, and one axial channel located parallel to a cylinder axis.

In another aspect of the present disclosure, which may be used in combination with any other aspect or combination of aspects listed herein, an axial channel connects the ducts of two adjacent biochips through connection ports.

In another aspect of the present disclosure, which may be used in combination with any other aspect or combination of aspects listed herein, radial channels reach halfway through the cylinder.

In another aspect of the present disclosure, which may be used in combination with any other aspect or combination of aspects listed herein, radial channels of each cylinder connect fluid channels from an operating system to an inlet or outlet of two adjacent biochips, while blocking flow between the two adjacent biochips.

In another aspect of the present disclosure, which may be used in combination with any other aspect or combination of aspects listed herein, an angle of rotation of cylinders allows either a flow to pass from one biochip duct to a duct in at least one adjacent biochip or to block the flow between the ducts of the at least one adjacent biochip and give access to operating system channels to the biochip ducts separately.

In another aspect of the present disclosure, which may be used in combination with any other aspect or combination of aspects listed herein, a small hole is located on a radial side of a cylinder in between two radial channels and is used to control an angle of rotating cylinders.

In another aspect of the present disclosure, which may be used in combination with any other aspect or combination of aspects listed herein, rotating cylinders maneuver in two positions.

In another aspect of the present disclosure, which may be used in combination with any other aspect or combination of aspects listed herein, position one comprises duct one outlet from a first biochip in alignment with a channel in a rotating cylinder reaching to a radial channel aligned with operating system channels.

In another aspect of the present disclosure, which may be used in combination with any other aspect or combination of aspects listed herein, a fluid passes from an operating system through a duct without reaching an adjacent biochip.

In another aspect of the present disclosure, which may be used in combination with any other aspect or combination of aspects listed herein, position two comprises duct one outlet from a first biochip in alignment with an axial channel in the cylinder, where fluid is able to pass through a rotating cylinder to an adjacent biochip.

In another aspect of the present disclosure, which may be used in combination with any other aspect or combination of aspects listed herein, a pneumatically actuated drilled piston micro-valve is provided.

In another aspect of the present disclosure, which may be used in combination with any other aspect or combination of aspects listed herein, a pneumatically actuated drilled piston micro-valve is equipped with two top inlet ports that allow fluid flow into the duct port, this valve can be pneumatically actuated through an air inlet one, air inlet two, and air inlet three, wherein each air inlet controls a piston that is actuated between an open/closed position.

In another aspect of the present disclosure, which may be used in combination with any other aspect or combination of aspects listed herein, once a valve is actuated, three pistons are configured to move into an open position wherein drilled holes are configured to align allowing fluid flow.

In another aspect of the present disclosure, which may be used in combination with any other aspect or combination of aspects listed herein, a set of membranes above and below a plurality of pistons are housed inside a valve housing.

In another aspect of the present disclosure, which may be used in combination with any other aspect or combination of aspects listed herein, membranes allow airflow to actuate pistons between open/closed positions.

In another aspect of the present disclosure, which may be used in combination with any other aspect or combination of aspects listed herein, at a bottom side of a pneumatically actuated drilled piston micro-valve, a duct port connects two adjacent biochips together.

In another aspect of the present disclosure, which may be used in combination with any other aspect or combination of aspects listed herein, a pneumatically actuated drilled piston micro-valve is provided. In another aspect, a pneumatic actuated open-close membrane valve, a ring and a top housing lock a top membrane in position, so when pneumatically actuated, the piston is pushed by the membrane to align holes of the piston and a microfluidic channel. In another aspect, a lower membrane retracts the piston to its normal position. In another aspect, a bottom housing and a middle housing lock the lower membrane in position. In another aspect, when the hole of the drilled piston aligns with the hole in the valve, it opens to allow fluid flow. In another aspect, if the piston hole is not aligned it blocks the flow.

In another aspect of the present disclosure, which may be used in combination with any other aspect or combination of aspects listed herein, a pneumatically actuated flexible tube micro-valve connecting adjacent biochip channels in a human-on-chip plate is provided.

In another aspect of the present disclosure, which may be used in combination with any other aspect or combination of aspects listed herein, a valving mechanism connecting individual biochip channels is bistable, wherein each of a plurality of valves contains four bistable beams, opening or closing a flexible tube. In another aspect, once actuated, the bistable beam will stay in one of those two bistable positions. In another aspect, the tube is placed in between an upper plate and a bottom plate, the bistable beam is located and interfaced with openings on each plate. In another aspect, a bottom balloon channel is pneumatically actuated, wherein when air flows in a channel, it closes all of a plurality of channels in a micro-valve. In another aspect, an upper set of singular balloons are individually pneumatically actuated, each one actuates the bistable beam below it, opening a portion of the tube that is below it. In another aspect, this mechanism is enclosed inside two covers, which may support its interface with the human-on-chip plate. In another aspect, the valve has four upper singular balloons and a normally set bottom balloon channel to allow for a multiplex valving system to be actuated with a minimum number of actuators.

In another aspect of the present disclosure, which may be used in combination with any other aspect or combination of aspects listed herein, a bistable pneumatically actuated magnetic drilled-piston micro-valve, connecting adjacent biochips of a human-on-chip plate is provided. In an embodiment, a valving mechanism is bistable through an upper magnetic plate and a lower magnetic plate. In another aspect, once actuated, holes of the drilled pistons will align with a channel inside the valve allowing fluid flow across the valve. In another aspect, the valve has four pistons to allow for a multiplex valving system to be actuated with a minimum number of actuators.

In another aspect of the present disclosure, which may be used in combination with any other aspect or combination of aspects listed herein, a bistable mechanically actuated flexible tube micro-valve connecting adjacent biochip channels of a human-on-chip plate through the microfluidic channels is provided. In another aspect, a valving mechanism is bistable, where the micro-valve has two stable positions. In another aspect, at an initial position of the bistable beam the flexible tube is open and a solenoid is not actuated. In another aspect, in a second position, the solenoid pushes a piston that actuates the bistable beam positions closing the flexible tube and blocking the flow in the channels. In another aspect, at least one pin holds the bistable beams in position. In another aspect, the valve components are contained inside the valve housing. In another aspect, the micro-valve can be solenoidally or pneumatically actuated in order to move the main beam.

In another aspect of the present disclosure, which may be used in combination with any other aspect or combination of aspects listed herein, a mechanically actuated pinched ball open-close micro-valve connecting the individual biochip components of the human-on-chip plate is provided. In another aspect, the valving mechanism connects individual biochip components and is either open or closed, wherein a ball micro-valve, contained in a ball valve housing, is a drilled sphere that can be rotated to either allow a fluid to pass or block it. In another aspect, the ball-micro-valve is mechanically actuated by a motor controlled by a system that can be actuated by a robot that moves in a 3D plane and opens or closes the ball-micro-valve using, optionally, a torx bit head, or in a standalone system that can preset the micro-valves configurations in order to mimic an organ in the human body.

In another aspect of the present disclosure, which may be used in combination with any other aspect or combination of aspects listed herein, different permanent seal open-close micro-valve connecting the individual biochip components of the human-on-chip plate are provided. In another aspect, the valving mechanism which connects individual biochip components through its cylindrical holes is either open or closed, wherein a permanent seal is preset once to either have an opened or closed micro-valve, wherein a pin is permanently inserted into the channel, wherein the pin can be either a blocking pin or an opening pin.

In another aspect of the present disclosure, which may be used in combination with any other aspect or combination of aspects listed herein, fluids, cells, and other biological components leaving a system may be sampled and aliquoted at any designed time-lapses, by pumping, opening and/or closing their respective valves. In another aspect, tubes coming out of the system are interfaced with various measurement techniques, such as Fourier-Transform Infrared (FTIR) Spectroscopy devices, O2 & CO2 sensing devices, and pH sensing devices placed along the tubes coming out of the system.

In another aspect of the present disclosure, which may be used in combination with any other aspect or combination of aspects listed herein, the human-on-chip plate includes a manifold that ensures an internal space of the system remains at specific environmental conditions that can be modulated for specific testing protocols. In another aspect, heat dissipaters or heat generators may be utilized to mitigate undesirable temperature conditions.

In another aspect of the present disclosure, which may be used in combination with any other aspect or combination of aspects listed herein, the human-on-chip plate includes a fluid control system which is able to deliver and/or collect the necessary reagents, at precise times and channels of one or more biochips within the operating system, required to carry out analytical assays. In another aspect, the assays may be measured with an inbuilt microscope, spectrometer and/or FTIR, or with any other appropriate analytical device either inbuilt or not.

In another aspect of the present disclosure, which may be used in combination with any other aspect or combination of aspects listed herein, a human-on-chip plate includes a fluid control system which is able to deliver and/or collect liquid samples from any channel in any biochip within an operating system. In another aspect, the liquid samples collected or delivered may contain any required chemical or biological material, which includes but is not limited to cells, enzymes, drugs, growth factors, nutrients, etc. In another aspect, a volume of the sample delivered to each channel is between 0.5 and 25 μL.

In another aspect of the present disclosure, which may be used in combination with any other aspect or combination of aspects listed herein, a human-on-chip plate comprises a fluid control system which is configured to pump liquid samples from any channel in any biochip within an operating system. In another aspect, the liquid samples may be pumped either from top inlet holes or from a side. In another aspect, fluids delivered to the plurality of the channels within each biochip and/or across different biochips within the operating system may be the same or different. In another aspect, gasses may also be pumped in one or a plurality of channels instead of a fluid. In another aspect, liquids or gasses may also be pumped at different flow rates. In another aspect, liquids being delivered may contain chemicals, drugs, polymers, biological materials, cells, growth factors, nutrients, or any other substance.

In another aspect of the present disclosure, which may be used in combination with any other aspect or combination of aspects listed herein, a human-on-chip plate includes a fluid control system which is able to deliver a polymerizing substance which may contain cells or other biological or chemical substances to any or multiple channels (stromal, and/or ductal) of one or more biochips within the operating system. In another aspect, the control system may be coupled with temperature controllers to ensure that a desired temperature is maintained such that the polymerizing substance is in its liquid state until it is delivered to a desired duct. In another aspect, once the polymerizing substance has been delivered, the temperature will be raised to ensure gelification.

In another aspect of the present disclosure, which may be used in combination with any other aspect or combination of aspects listed herein, the assembled human operating system is a benchtop analytic operation device that consists of multiple interconnected subsystems. In another aspect, the system allows tissue culture of different organs within different arrangements, to mimic a human body. In another aspect, the microfluidic system designed in the assembled human operating system is configured to exchange any fluids in the system. In another aspect, the assembled human operating system may comprise a cover to preserve subsystems inside from any outside intervention, and a machine frame that holds all components together. In another aspect, the assembled human operating system may be equipped with an electrical enclosure that powers the entire system. In another aspect, biochips may be assembled on the human-on-chip plate which may be installed in the system using a sliding mechanism. In another aspect, a three-axis robot is configured to move freely in 3-dimensional space above any installed biochip. In another aspect, the system is equipped with multiple measuring and sensing devices that are fixed on the three-axis robot, the measurement and sensing devices used may include, but are not limited to, a spectrophotometer, oxygen and carbon dioxide sensor, and a microscope. In another aspect, the spectrophotometer uses biological assays to measure the cell's viability and functionality inside the biochips. In another aspect, the O2 and CO2 sensor is used to monitor dissolved gas concentration to maintain favorable conditions for cell seeding and proliferation or to measure induced hypoxic conditions. In another aspect, the microscope is used to examine and analyze morphology of cells inside the biochips. In another aspect, a fluidic operation system is configured to feed and exchange fluids inside the system using the microfluidic channels. In another aspect, a media storage compartment is configured to house discarded media after its use. In another aspect, ATR-FTIR is a measuring device to measure a composition of a molecule in a media sample. In another aspect, a mixing table contains the mixing chamber, which may house the cells and media that will be used by the human operating system.

In another aspect of the present disclosure, which may be used in combination with any other aspect or combination of aspects listed herein, a fluidic operating system includes ports that connect a human operating system with tubes that move waste fluid out of a biochip into a waste chamber, through waste chamber ports. In another aspect, the waste fluid is controlled by pneumatically actuated valves via inlets. In another aspect, inlet ports connect coolant or heated fluid between the fluidic operating system to a heat exchanger located above the human-on-chip plate. In another aspect, the human operating system is connected by ports with a pneumatic air signal inlet that is configured to actuate the valves allowing heat controlled fluid to pass via the inlets. In another aspect, the human operating system is equipped with media microchambers, that store media in a close proximity to the biochips. In another aspect, each media microchamber stores media and culture components that feed into individual microchannels within each biochip. In another aspect, the media microchambers are connected to the fluidic operating system through media microchambers inlet ports. In another aspect, the valves control seventy-two media microchambers and are actuated via sixteen multiplexed signals through the pneumatic air ports. In another aspect, thirty-three valves connecting the adjacent biochip microchannels within the human-on-chip plate are pneumatically actuated by eleven multiplex signals through the pneumatic air ports.

In another aspect of the present disclosure, which may be used in combination with any other aspect or combination of aspects listed herein, a fluidic operating system contains four ports that control different sensors located on a human-on-chip plate. In another aspect, temperature sensors are controlled by electric signals coming from an electrical enclosure through a temperature sensors port. In another aspect, flow and pressure sensors relay a feedback signal to the electrical enclosure through a flow and pressure sensors port. In another aspect, O2 sensors relay a feedback signal to the electrical enclosure through an O2 sensors port, and CO2 sensors relay a feedback signal to the electrical enclosure through a CO2 sensors port.

In another aspect of the present disclosure, which may be used in combination with any other aspect or combination of aspects listed herein, a media micro chamber is provided located in close proximity with respect to a human-on-chip, wherein decreasing the distance between the two different components allows the use of a few microliters of fluid, reducing a possibility of diluting samples. In another aspect, a 3/3 spring-loaded pneumatically actuated valve controls a flow of fluid from a mixing chamber into the media microchamber. In another aspect, a fluid will travel into a 72-channel manifold. In another aspect, each channel in the manifold leads to a single media micro-chamber that's equipped with an open-close gait allowing or restricting the fluid flow into the media microchamber.

In another aspect of the present disclosure, which may be used in combination with any other aspect or combination of aspects listed herein, an electro-mechanically controlled robotic arm is embedded and interlinked to an operating system, and may have at least one built-in pipette that can take samples from at least one sampling spot on the human-on-chip plate.

In another aspect of the present disclosure, which may be used in combination with any other aspect or combination of aspects listed herein, a human-operating-system is provided. In another aspect, a mixing chamber is divided into two main compartments that handle cells and media. In another aspect, for the cells, a pressure unit is connected to the cell chamber. In another aspect, a pressure unit will provide the cells with necessary gases for cell survival and move the cells from the cell chamber to a human-on-chip plate. In another aspect, the cells will pass through a 12/1-way valve into a flow meter, air trap, and pressure sensor that will monitor and report to the system. In another aspect, for the media, a pressure unit is connected to the media chamber which will be connected to a 4/1-way valve. In another aspect, the media will be monitored by a flow meter, air tap, and pressure sensor. In another aspect, both cells and media pass through a quick connect into a 3/3 spring-loaded pneumatically actuated valve that is configured to regulate cell and media flow into the biochip. In another aspect, in the human operating system assembly, a pneumatically actuated piston is configured to pump the mixture into a media chamber located in close proximity to the biochip. In another aspect, the system can control the mixture direction using a 3/3 spring-loaded pneumatically actuated valve that can direct the mixture either to a waste chamber or to a set of 3 2/2 bistable pneumatically actuated valve that operates in a pre-determined logic that allows the mixture to be sent to a first duct, second duct, stroma or all three sites at once.

In another aspect of the present disclosure, which may be used in combination with any other aspect or combination of aspects listed herein, a human operating system assembly contains measuring devices. In another aspect, the measuring devices comprise a microscope/spectroscope/O2 and CO2 assembly and an ATR-FTIR unit. In another aspect, the human operating system assembly is equipped with a sampling site and a 3-axis robotic external sampling pipette that can be used to extract samples from the system.

In another aspect of the present disclosure, which may be used in combination with any other aspect or combination of aspects listed herein, a human operating system is provided, wherein system components are stacked over each other in layers. In another aspect, an assembly of the human operating system is covered by a protective cover with a base plate that's held in place using bolts. In another aspect, a human-on-chip plate will be fed into the system from a side and a spectroscope sensing plate is located under the human-on-chip plate with a spectroscopy source plate on top. In another aspect, the human operating system comprises 12 stacked layers with each layer providing a certain set of functionalities, wherein the spectroscope source plate situated on top of the device is used as a measuring device to monitor cell functionality. In another aspect, underneath that, a top media sourcing plugs plate that stores media is provided and a top open-close valve plate controls media flow. In another aspect, a piston actuator plate and piston plate pump media from the top media source plugs plate into the micro media chamber plate. In another aspect, the media will go through an upper open/close valve plate, middle direction shift valve plate, and lower open/close valve plate that are configured to change a direction of the media, sending it to a specific biochip. In another aspect, an inlet/outlet valve actuator plate is configured to be a final media control point that allows the media to flow into/out of a biochip that is located on the human-on-chip plate. In another aspect, a spectroscope sensing plate will receive emitted light from a spectroscopy source plate through the biochip found on the human-on-chip plate. In another aspect, the spectroscopy source plate is configured to move aside allowing a microscopic array plate to have a clear line of sight to perform microscopic imaging of at least one cells culture in the biochip.

In another aspect of the present disclosure, which may be used in combination with any other aspect or combination of aspects listed herein, a fluid pumping control system is provided wherein a syringe pump is connected to a top open/close valve plate via connection tubing. In another aspect, the fluid pumping control system controls an amount of media that is available to be used in a cell culturing stage. In another aspect, the media is stored in media storage, and using syringe pumps the media can be withdrawn and transferred to a micro media chamber plate that is placed on top of a human-on-chip plate. In another aspect, the media will be situated directly on top of the cultured cells in the biochips of the human-on-chip plate. In another aspect, a set of pistons are fixed on a piston plate sandwiched between a piston actuator plate and the micro media chamber plate. In another aspect, the piston actuating plate is configured to control the pistons extensions and retraction movement allowing a small amount of media to flow into the micro media chamber plate. In another aspect, in between the micro media chamber plate and human-on-chip plate a valving plate is placed, wherein the valving plate works alongside the inlet/outlet valve actuator plate to control fluid flow in and out of the human-on-chip plate.

In another aspect of the present disclosure, which may be used in combination with any other aspect or combination of aspects listed herein, the human operating system is equipped with a pneumatic actuator container, FTIR machine, media storage container, and microscope array. In another aspect, a pneumatic actuators container can be used to control multiple components of the system wherein the pneumatic actuators container can be used as a pressure source to drive media from a media storage container into a micro media chamber plate passing through a top media source plugs plate that is configured to transfer a few microliters of fluids. In another aspect, after the fluid quantity is reduced to a few microliters, a set of pistons capable of handling such quantities is fixed on a piston actuator plate that can be pneumatically controlled by the pneumatic actuators container linked to a pneumatic actuating valve fitting plate to derive media or other fluids from the media storage container in the micro media chamber plate that is situated on top of the human-on-chip plate that houses the biochips where tissues will be cultured. In another aspect, the human operating system is equipped with a triple measuring system comprising a spectroscope sourcing plate emitting a signal to a spectroscope sensing plate. In another aspect, the cultured tissues are also interlined with real-time FTIR using sampling channels where a fluid sample can be excreted to be tested. In another aspect, both work together to gather data about an effect that a drug exerts on the tissues. In another aspect, a microscope array consisting of multiple microscope lenses is placed underneath the human-on-chip plate. In another aspect, all three systems will be used to track the biological assays that will indicate the cell's morphology, viability, and drug efficacy.

In another aspect of the present disclosure, which may be used in combination with any other aspect or combination of aspects listed herein, a micro media chamber is placed on top of a human-on-chip plate.

In another aspect of the present disclosure, which may be used in combination with any other aspect or combination of aspects listed herein, pistons and a piston plate are used to transfer fluids from a media storage container into a micro media chamber. In another aspect, the pistons are configured to alternate between an extended and retracted position to move the fluid. In another aspect, the pistons can be actuated mechanically, fluidically, electrically, or pneumatically.

In another aspect of the present disclosure, which may be used in combination with any other aspect or combination of aspects listed herein, a piston shaft goes into a cylindrical cut-out that is configured to receive fluid from the media storage container through an inlet port on a side of a piston housing. In another aspect, when piston is in its retracted position the fluid is sucked into the cylindrical cut-out through the inlet port on the side of the piston housing, the piston is actuated causing it to move to its retracted position covering the side inlet port and compressing the fluid forcing it to be transferred into the micro media chamber. In another aspect, on a bottom side, the actuator shaft will be connected to the piston cylindrical cutout which actuates the pistons to their open and closed state by switching between the extended and retracted position of the actuator shaft.

In another aspect of the present disclosure, which may be used in combination with any other aspect or combination of aspects listed herein, a top media source plugs plate of the human operating system is provided, wherein the system is equipped with a set of syringe pumps that is connected to a pistons inlet port via connection tubes. In another aspect, the syringe pumps can be replaced with other forms of fluid pumping like pressure-driven pumps.

In another aspect of the present disclosure, which may be used in combination with any other aspect or combination of aspects listed herein, an interface of an operating system with a biochip in a human-on-chip is provided wherein the system has different levels of valves that can be actuated in multiple different ways separately. In another aspect, a first level of valves is configured to control the media and other fluid flow into the media chamber, a second level of valves control the fluid and media flow into the biochip or a waste chamber, and a third level controls the fluid and media leaving the biochip, wherein the media leaving the biochip can be induced into another one or multiple biochips, wherein valves can operate in different open/close positions driving the fluids into a different location.

In another aspect of the present disclosure, which may be used in combination with any other aspect or combination of aspects listed herein, an initiation process of the human-on-chip is provided where a sequence of preprogrammed instructions will test for any failure in the valve actuation, then the media or other fluids will be pumped from the mixing chamber into a 2/2 bistable pneumatically actuated valve that's switched into its open position allowing media to pass into a pneumatically actuated piston that will extend and retract to force the media into the media chamber. In another aspect, this comprises a priming stage that will follow the initiation process. In another aspect, a 3/3 spring-loaded pneumatically actuated valve is in a closed position to prevent the fluid from escaping into a biochip or a waste chamber while the media chamber is being filled.

In another aspect of the present disclosure, which may be used in combination with any other aspect or combination of aspects listed herein, steps after a priming stage are provided where a 2/2 bistable pneumatically actuated valve is switched into a closed position preventing media from being forced back into a mixing chamber by a pneumatically actuated piston. In another aspect, the 3/3 spring-loaded pneumatically actuated valve then switches into an opened position directing fluid flow into a waste chamber, and the pneumatically actuated piston is actuated to push the fluid into the waste chamber.

In another aspect of the present disclosure, which may be used in combination with any other aspect or combination of aspects listed herein, a human-on-chip after a flushing stage is provided, wherein media is pumped into a media chamber from a mixing chamber through a 2/2 bistable pneumatically actuated valve by a pneumatically actuated piston. In another aspect, a 3/3 spring-loaded pneumatically actuated valve is in an opened position directing a flow into a biochip. In another aspect, a 3/3 spring-loaded pneumatically actuated valve is placed on a biochip outlet to control a fluid coming out of the biochip, wherein this valve can direct the fluid to a waste chamber or to another biochip on the human-on-chip plate.

In another aspect of the present disclosure, which may be used in combination with any other aspect or combination of aspects listed herein, a human-on-chip system is equipped with an air trap. In another aspect, the air trap is configured to allow manipulation of gas content in a media without formation of bubbles inside the system. In another aspect, each biochip is connected to two different assemblies of the media chamber, valves, and waste chamber, wherein such arrangement is configured to allow control of fluids going in/out, and a type of fluid going into ducts and a stroma, or it is configured to allow each biochip to derive fluid from a separate media chamber through setting a 3/3 spring-loaded pneumatically actuated valve in a certain open/close position.

In another aspect of the present disclosure, which may be used in combination with any other aspect or combination of aspects listed herein, multiple biochips are connected to multiple media sources, all interlinked by a valving system. In another aspect, the human operating system will consist of a human-on-chip plate which will house twelve different biochips capable of hosting multiple cell cultures. In another aspect, a cell culture may comprise a single cell type or may comprise a co-culture of at least one different cell type, seeding in a stroma and ducts of the biochip. In another aspect, a sample of fluid can be extracted from a biochip one and transferred to a different biochip on the human-on-chip plate.

In another aspect of the present disclosure, which may be used in combination with any other aspect or combination of aspects listed herein, a plurality of measuring and sensing devices are provided, each measuring and sensing device characterized by certain functionality that a system will rely on to monitor and maintain cultured tissues and configured to measure cell reaction, efficacy, or cytotoxicity of an introduced drug. In another aspect, a sampling site can be accessed manually by technicians or automatically using a 3-axis robotic external sampling pipette.

In another aspect of the present disclosure, which may be used in combination with any other aspect or combination of aspects listed herein, an operation of the human operating system is provided wherein cells are extracted from a cell mixing chamber into a quick connect that connects a human-on-chip plate to a human operating system. In another aspect, a 3/3 spring-loaded pneumatically actuated valve may comprise three functions, first of which is to allow the flow of the cell into the human-on-chip plate, second of which is the valve can switch into a second position allowing the media flow, and the third of which is a closed position which will prevent anything from going in or out of the human-on-chip plate. In another aspect, an air trap mediates the 3/3 spring-loaded pneumatically actuated valve and a pneumatically actuated piston. In another aspect, the fluid is able to flow into the media chamber. In another aspect, a 2/2 bistable pneumatically actuated valve may be connected to a channel in between the media chamber and the pneumatically actuated piston. In another aspect, based on the position of the valve, fluid can either flow to the media chamber or can travel to the next junction. In another aspect, this arrangement allows sampling out of either a single biochip or from multiple biochips without the need for a sample to pass through all biochips found on the human-on-chip plate. In another aspect, the 3/3 spring-loaded pneumatically actuated valve may be located underneath the media chamber and control a flow direction by either blocking the flow, diverting the flow to a waste chamber to be excreted out of the system or allowing the flow to go into a biochip for flushing, cell seeding, and refeeding.

In another aspect of the present disclosure, which may be used in combination with any other aspect or combination of aspects listed herein, a human operating system comprises four biochips wherein a valving system connects the four different biochips to each other and to different components of the human operating system. In another aspect, a measuring and sensing assembly that comprises microscope/spectroscope/O2 sensor/CO2 sensor assembly is configured to monitor a cultured tissue before and after introducing at least one drug. In another aspect, an operator can choose to extract a sample where the different valves will switch into an open or closed state in which they are configured to create a pathway for the sample to travel to reach a set of 2/2 bistable pneumatically actuated valves that can control a direction of the sample either to an integrated ATR-FTIR or to a sampling site wherein a 3-axis robotic external sampling pipette can move over and extract the sample to be studied and analyzed outside the system. In another aspect, the sample travels into the ATR-FTIR, and wherein, once that measurement is done, the sample is ejected into a waste chamber. In another aspect, the sample may be ejected out to the waste chamber through a 2/2 bistable pneumatically actuated valve without going into the ATR-FTIR.

In another aspect of the present disclosure, which may be used in combination with any other aspect or combination of aspects listed herein, a process of injecting biological gel into a biochip is provided. In another aspect, a dedicated stroma gel inlet and stroma gel outlet may be implemented. In another aspect, a double-headed pipette may be used. In another aspect, pipette tips used are spaced to align with the stroma gel inlet and outlet. In another aspect, one side of the pipette is configured to pump the gel into the biochip and the other is configured to pump excess gel out of the biochip. In another aspect, gel ports are equipped with a biocompatible self-healing membrane that prevents exposure of the gel to the outer atmosphere and causes potential contamination.

In another aspect of the present disclosure, which may be used in combination with any other aspect or combination of aspects listed herein, a mixing chamber is provided. In another aspect, the mixing chamber houses cells and media to be used by a human operating system. In another aspect, the mixing chamber may comprise cell mixing chamber and media mixing chamber. In another aspect, the cell mixing chamber will store the cells in favorable conditions which will then be used by the human operating system to be seeded in a biochip in a human-on-chip plate. In another aspect, the media mixing chamber contains media used to maintain cultured tissues for a prolonged period of time. In another aspect, both chambers are equipped with a gas source that can induce different types of gases to mimic different conditions or to maintain similar conditions as in the human body. In another aspect, the cell mixing chamber may contain three rows of cell chambers wherein each row is made up of 12 individual cell chambers, wherein an assembly of 36 cell chambers forms the cell mixing chamber. In another aspect, an array of 12 media chambers may be interlinked into each other forming the media mixing chamber.

In another aspect of the present disclosure, which may be used in combination with any other aspect or combination of aspects listed herein, a pressure/gas source assembly is provided, wherein two units, one for the media mixing chamber and one for the cell mixing chamber, act as a pressure source to supply a system with a pressure needed to pump fluids from a mixing chamber to a human-on-chip plate. In another aspect, the pressure/gas source assembly is divided into two sections housed in the pressure/gas container, the first section may be a pressure/vacuum source and the second section may be a gas source. In another aspect, each of the media mixing chamber and cell mixing chamber comprise 3 rows of components, and the pressure/vacuum source accommodates this arrangement by comprising 3 different pressure outputs that may be controlled individually. In another aspect, the gas source is configured to supply cells and media with a necessary gas mixture to maintain favorable conditions for tissue culture. In another aspect, the cell mixing chamber is equipped with 36 gas output while the media mixing chamber have 12 gas output that supplies the needed gas mixture.

In another aspect of the present disclosure, which may be used in combination with any other aspect or combination of aspects listed herein, an individual media chamber assembly is provided wherein the media chamber will store media to be later used for referring the tissue culture. In another aspect, a gas output from a pressure/gas assembly is connected to a media gas input on the media chamber. In another aspect, an O2/CO2 sensor is placed underneath to monitor the dissolved gas concentration in the media, and wherein based on this reading the system will regulate the amount of gas being perfused. In another aspect, a media sampling port is implemented on the top cover of media chamber, and wherein this port can be accessed manually by an operator to perform a regular quality test on the media. In another aspect, a media pressure input is connected to the pressure/vacuum source, wherein the pressure will drive the media from the media chamber to the human-on-chip plate. In another aspect, the media chamber is equipped with a media output port wherein the media will pass through the port to the tubing and then to the human-on-chip plate.

In another aspect of the present disclosure, which may be used in combination with any other aspect or combination of aspects listed herein, an individual media chamber connected to the media mixing chamber is provided. In another aspect, an assembly starts by connecting a pressure/gas source assembly to a media chamber assembly. In another aspect, connection lines connect a pressure and gas output from the pressure/gas source to a pressure and gas input in the media chamber. In another aspect, media will be supplied with a required gas mixture, and pressure will drive fluids from the media chamber assembly through the media output port to a 4/1-way valve. In another aspect, the 4/1-way valve rotates between four different positions. In another aspect, if a valve spool clicks in one of these positions, two openings will align allowing fluid to flow from the media chamber through the valve into a flow meter. In another aspect, a flow meter is used to monitor a fluid flow and acts as a feedback signal for the system. In another aspect, the flow meter is used to control the flow. In another aspect, an air trap is placed directly after the flow meter, which is configured to reduce a possibility of an air bubble escaping into the fluidic operating system. In another aspect, a media pressure gauge will monitor the fluid pressure, and both sensors will provide feedback signals measuring the flow rates or detecting the presence of any blockage in the tubing. In another aspect, the fluid goes through the tubing into a quick connect that will connect the mixing chamber to the fluidic operating system.

In another aspect of the present disclosure, which may be used in combination with any other aspect or combination of aspects listed herein, a cell mixing chamber is provided, wherein each individual cell chamber assembly is directly connected to a quick connector which is interim connected to a fluidic operating system. In another aspect, a pressure/gas source assembly pressurizes the cell chamber causing cells to flow into a human-on-chip plate passing through the fluidic operating system.

In another aspect of the present disclosure, which may be used in combination with any other aspect or combination of aspects listed herein, a mixing chamber comprises a cell mixing chamber and a media mixing chamber and quick connectors used to link the mixing chamber with a fluidic operating system.

In another aspect of the present disclosure, which may be used in combination with any other aspect or combination of aspects listed herein, a cell chamber assembly is provided where the cells will be stored to later be used in the system. In another aspect, the cell chamber is equipped with a gas input port that is configured to connect to a pressure/gas source, and an O2/CO2 sensor is configured to monitor a gas concentration in the cell chamber and then relay the feedback to an operating system for any adjustments. In another aspect, the cell chamber is equipped with a sourcing port that can be used to inject cells into cell chamber or to take a test sample. In another aspect, a pressure line is configured to be connected from the pressure/vacuum source to the cell pressure input on the cell chamber. In another aspect, pressure will be used to pump the cells from the cell chamber to a human-on-chip plate.

In another aspect of the present disclosure, which may be used in combination with any other aspect or combination of aspects listed herein, a connection for a pressure source of a pressure/gas source assembly into a cell chamber pressure input is provided. In another aspect, the cell chamber assembly comprises 36 cell chambers that form the cell mixing chamber.

In another aspect of the present disclosure, which may be used in combination with any other aspect or combination of aspects listed herein, each row of a cell chamber assembly forms a set of cell chambers. In another aspect, a pressure/gas assembly is connected to the cell chamber assembly, supplying it with a necessary gas mixture to maintain cell viability. In another aspect, another connection point is from a pressure output of the pressure/gas assembly to a cell pressure input. In another aspect, pressure will drive the cells from the cell chamber to a 12/1-way valve. In another aspect, the 12/1-way valve rotates between 12 different positions, and 12 cell chambers assembly are connected to this valve. In another aspect, once the valve rotates in one of the 12 positions, two port openings inside the valve assembly align allowing fluids to pass through. In another aspect, a mixture of fluid and cells travels through connection tubing passing by detection sensors. In another aspect, the detection sensor comprises a flow meter and a cell pressure sensor. In another aspect, the sensors monitor a flow rate of the fluid mixture and provide feedback to a control system. In another aspect, this arrangement allows the detection of issues comprising clogging or blockage inside micro-channels. In another aspect, the fluid mixture will go through a quick connector into a fluidic operating system.

In another aspect of the present disclosure, which may be used in combination with any other aspect or combination of aspects listed herein, the connections between the pressure/gas source and the media mixing chamber are provided. In another aspect, the pressure/gas source assembly supplies the media mixing chamber with the necessary pressure through a media pressure input. In another aspect, the pressure/gas source will drive media from the media chamber to a fluidic operating system. In another aspect, gases that are perfused in the media with other nutrients are supplied by the gas output on the pressure/gas source.

In another aspect of the present disclosure, which may be used in combination with any other aspect or combination of aspects listed herein, oxygen and carbon dioxide sensors are provided. In another aspect, multiple sensing devices may be implemented in a system to trace back any fluctuations in the gas mixture. In another aspect, a first sensing level is in the mixing chamber wherein each individual cell chamber and media chamber is equipped with an O2/CO2 sensor configured to measure and monitor a gas concentration. In another aspect, the second sensing level is through a chip embedded O2/CO2 sensor that is configured to perform a continuous real-time measurement. In another aspect, data collected by the chip embedded O2/CO2 sensor provides significant information about cell viability and functionality, especially in the case of testing tumor invasion and growth inside the biochip. In another aspect, when an experiment requires hypoxic conditions, and such condition is needed in specific biochips the chip embedded O2/CO2 sensor will provide an exact and reliable reading in each specific biochip. In another aspect, to support the chip embedded O2/CO2 sensor reading, a 3-axis robotic external O2/CO2 sensor is implemented. In another aspect, the sensor is configured to move in 3-dimensional space over the biochips found on a human-on-chip plate. In another aspect, the 3-axis robotic external O2/CO2 sensor is configured to work as an overall independent sensor, wherein obtained data is cross-referenced with a reading from the Chip embedded O2/CO2 sensor to eliminate any false measurements. In another aspect, the system relies on this data to perform any modification in the gas mixture provided by the pressure/gas source.

In another aspect of the present disclosure, which may be used in combination with any other aspect or combination of aspects listed herein, a heating and cooling process on the human-on-chip plate is provided. In another aspect, a heat exchange unit is connected to a fluidic operating system via tubings that are configured to circulate heating and cooling fluid. In another aspect, the fluid will pass through the fluidic operating system into a human-on-chip plate, wherein one of a plurality of layers of this plate is a heating plate. In another aspect, the heating plate is machined from the inside to create pathways that the cooling and heating fluid can pass through to alter a temperature of the human-on-chip plate.

In another aspect of the present disclosure, which may be used in combination with any other aspect or combination of aspects listed herein, a system initiation process is provided, wherein a system will run a self-diagnosis sequence. In another aspect, a first stage in this sequence is to test functionality of any valves by actuating them between their open/closed position. In another aspect, if any valve reports an error, an operator may be notified. In another aspect, once valve functionality is confirmed, the system will initiate a flushing process of all components. In another aspect, this step may include washing the valves and any microchannels, removing any contaminants or debris from the system, and verifying that none of the valves or microchannels are blocked. In another aspect, a final initiation step is to circulate media in the system and to start a heating process in preparation for cell seeding. In another aspect, O2/CO2 sensors measure a concentration of a gas mixture confirming the initiation for cell seeding.

In another aspect of the present disclosure, which may be used in combination with any other aspect or combination of aspects listed herein, different temperature sensors found in the system are provided. In another aspect, media and cells are stored in a mixing chamber. In another aspect, initial warm-up of a mixture begins. In another aspect, after the media reaches a desired temperature, it is transferred from the media mixing chamber into a biochip. In another aspect, the media will pass by at least one 3/3 spring-loaded pneumatically actuated valve into the media chamber. In another aspect, the media chamber is equipped with a media chamber temperature sensor that will report back to a system, forming a feedback control to maintain the desired temperature. In another aspect, the 3/3 spring-loaded pneumatically actuated valve switches to an open position, allowing the media to flow into a 2/2 bistable pneumatically actuated valve, wherein a set of three valves controls whether a flow will go into a first duct, second duct, stroma channel or into all at the same time. In another aspect, a chip embedded O2/CO2 sensor and 3-axis robotic external O2/CO2 sensor may measure the gas concentration. In another aspect, the same or a similar process may take place while injecting cells from a cell mixing chamber into a human on-chip plate. In another aspect, a final stage of temperature sensing takes place on the biochip directly. In another aspect, each biochip is equipped with an embedded temperature sensor that monitors the temperature internally. In another aspect, a thermal camera fixed to the 3-axis robot is used to image the human-on-chip plate for any cold or hot spots, wherein the camera also helps to assess the overall temperature of the plate.

In another aspect of the present disclosure, which may be used in combination with any other aspect or combination of aspects listed herein, a human operating system comprises embedded measurement devices that comprise a spectroscope array and a microscope array. In another aspect, the spectroscope array comprises a spectroscope sensing plate that is placed underneath a human-on-chip plate, wherein the sensing plate receives emitting light from the spectroscope array through a biochip. In another aspect, the sensing plate can move to the side, providing a clear sight for microscope lenses to image tissue culture. In another aspect, the light source needed by the microscope can be replaced by the emitted light from the spectroscope.

In another aspect of the present disclosure, which may be used in combination with any other aspect or combination of aspects listed herein, the human operating system comprises measuring devices fixed on a 3-axis robot. In another aspect, the measuring devices comprise a microscope equipped with a multi-lens turret that can rotate to switch between different magnification levels. In another aspect, next to the microscope is a 3-axis robotic external O2/CO2 sensor and a spectroscope. In another aspect, all the measuring devices can hover over a human-on-chip plate and perform analytical measurements.

In another aspect of the present disclosure, which may be used in combination with any other aspect or combination of aspects listed herein, a system that provides for at least two different tissues to be grown in contact via a duct or ducts (vascular or lymphatic vessel or both) is provided. In another aspect, this also allows for tissue-tissue interaction studies.

In another aspect of the present disclosure, which may be used in combination with any other aspect or combination of aspects listed herein, the biochip is capable of hosting an immune system model wherein an extracellular matrix is injected into a stromal channel of a biochip. In another aspect, the extracellular matrix forms a biocompatible environment that increases the stromal tissue attachment affinity. In another aspect, inside a ductal channel endothelial cells will be seeded, covering a perforated 3D scaffolding system. In another aspect, the ductal channel is loaded with immune cells, which travel across the duct and may diffuse through the endothelial cells layer to the stromal channel.

In another aspect of the present disclosure, which may be used in combination with any other aspect or combination of aspects listed herein, a biochip can host endothelial cells and pericytes inside a ductal channel. In another aspect, immune cells are injected into the duct. In another aspect, using onboard real-time microscope imaging, immigration of the immune cell to the stroma can be monitored and studied. In another aspect, the migrated immune cells may bypass the astrocytes and act on the stromal tissue forming Glioblastoma with immune cell infiltration.

In another aspect of the present disclosure, which may be used in combination with any other aspect or combination of aspects listed herein, a third measuring device may be an ATR-FTIR that may be integrated into the human operating system. In another aspect, each individual microchannel may be equipped with an ATR-FTIR, allowing the system to monitor duct channel and stroma channel individually. In another aspect, the multiple ATR-FTIR can be replaced by one ATR-FTIR placed at the end of the system.

In another aspect of the present disclosure, which may be used in combination with any other aspect or combination of aspects listed herein, an ATR-FTIR is placed in a separate dedicated unit inside a human operating system. In another aspect, tubing routes a sample of fluid to the ATR-FTIR.

In another aspect of the present disclosure, which may be used in combination with any other aspect or combination of aspects listed herein, biochip interconnections are provided. In another aspect, the biochip interconnections have a wide range of flexibility that provides the ability to freely connect biochips in a human-on-chip plate. In another aspect, multi-organs circulatory system is mimicked by connecting several biochips in a closed loop system, wherein a first biochip duct one outlet is connected to a next biochip duct one inlet and a last biochip duct one outlet is connected to a first biochip duct one inlet. In another aspect, a single organ circulatory system is mimicked by connecting duct two outlet to duct two inlet of the same biochip. In another aspect, a biochip is connected to several biochips through the first biochip's duct one inlet and duct one outlet to mimic a multi-organs circulatory system, while also having the biochip's own single organ circulatory system through the biochip's duct two inlet and duct two outlet. In another aspect, a biochip is connected to several biochips through the first biochip's duct one inlet and duct one, to mimic a multi-organs circulatory system, while also having coculture circulatory system through the biochip's duct two inlet and duct two outlet. In another aspect, multi-organs circulatory system that is connected to the media chamber outlet and the operating system is mimicked by connecting the first biochip duct one inlet to a media chamber and the duct one outlet to the next biochip duct one inlet, and the last biochip duct one outlet is connected to an operating system for sampling or to waste. In another aspect, a single organ circulatory system is mimicked by connecting duct two outlet to duct two inlet of the same biochip. In another aspect, a stroma in each biochip is controlled separately, or stromas can be connected in series or parallel.

In another aspect of the present disclosure, which may be used in combination with any other aspect or combination of aspects listed herein, a biological system-on-chip plate includes a plurality of ducted biochips, fluidic channeling means in fluidic communication with at least one biochip. In another aspect, the fluidic channeling means comprises a plurality of fluidic pathways and selectable connection means providing the ability to selectively connect or disconnect fluidic communication throughout a plurality of human-on-chip biochips. In another aspect, the ducted biochips have inlet and outlet ports. In another aspect, the ports of at least two biochips are fluidically connected to at least one fluidic pathway. In another aspect, fluidic channeling means enables transfer or circulation of media through ducts or stromal regions of multiple biochips. In another aspect, the selectable connection means enables arrangement of biochips in series or parallel as required for a particular experiment or application. In another aspect, at least one selectable connection means is configured to enable a permanent selection process to either connect or disconnect fluidic communication therethrough. In another aspect, at least one selectable connection means includes valving means that provide reversible connection and disconnection. In another aspect, the valving means enable isolation of a biochip during an experiment.

In another aspect of the present disclosure, which may be used in combination with any other aspect or combination of aspects listed herein, a single biochip is connected in a double circulatory system, wherein the two circulatory systems are independent of each other. In another aspect, a first duct outlet is connected to a first duct inlet forming a first loop, then a second duct outlet is connected to a second duct inlet forming a second loop. In another aspect, the fluid is in a closed circulation loop.

In summary, the human operating system is a self-enclosed system where multiple cell types can be grown and connected with each other, and experiments can be automatically run within the system. The aim of the system is to be used for tissue modeling and drug testing for the better prediction of the effects of diseases, biological parameters, and drugs before testing on humans. This allows for the standardization of cell culture conditions to ensure physiological conditions to an unprecedented level. It also ensures real-time and end-point experiments to be carried out within the system. The presently disclosed system allows for an all-in-one approach that ensures standardization, reproducibility, accuracy, speed, and reduced costs.

### BRIEF DESCRIPTION OF THE FIGURES

Some example apparatus embodiments of the present disclosure, and example procedures for making and using one or more example embodiments, are described in detail herein and by way of example, with reference to the accompanying drawings (which are not necessarily drawn to scale with regard to any internal or external structures shown) and in which like reference characters designate like elements throughout the several views, and in which:

FIG. 1 illustrates an isometric view of two ducts surrounded by one stroma, according to an example of the present disclosure.

FIG. 2A illustrates an isometric view of an embodiment of a 2-ducts 1-stroma biochip, according to an example of the present disclosure.

FIG. 2B illustrates a top view of an embodiment of a 2-ducts 1-stroma biochip, according to an example of the present disclosure.

FIG. 2C illustrates an isometric view of the manufacturing and assembly process of a 2-ducts 1-stroma biochip, according to an example of the present disclosure.

FIG. 3A illustrates an isometric view of another embodiment of a 2-ducts 1-stroma biochip with three parallel channels, according to an example of the present disclosure.

FIG. 3B illustrates an exploded view of a 2-ducts 1-stroma biochip with three parallel channels, according to an example of the present disclosure.

FIG. 3C illustrates an isometric view of the manufacturing and assembly process of an embodiment of a 2-ducts 1-stroma biochip with three parallel channels, according to an example of the present disclosure.

FIG. 4A illustrates an isometric view of another embodiment of a 2-ducts 1-stroma biochip with three parallel channels with a stroma feeding channel, according to an example of the present disclosure.

FIG. 4B illustrates a detailed top and bottom view of a 2-ducts 1-stroma biochip with three parallel channels with a stroma feeding channel, according to an example of the present disclosure.

FIG. 4C illustrates an exploded view of the embodiment of a 2-ducts 1-stroma biochip with three parallel channels with a stroma feeding channel, according to an example of the present disclosure.

FIG. 4D illustrates an isometric view of the manufacturing and assembly process of the 2-ducts 1-stroma biochip with three parallel channels with a stroma feeding channel, according to an example of the present disclosure.

FIG. 5A illustrates an isometric view of a 2-ducts 1-stroma biochip with different stroma inlets, according to an example of the present disclosure.

FIG. 5B illustrates a top view of another embodiment of a 2-ducts 1-stroma biochip with different stroma inlets, according to an example of the present disclosure.

FIG. 5C illustrates an exploded view of a 2-ducts 1-stroma biochip with different stroma inlets, according to an example of the present disclosure.

FIG. 5D illustrates an isometric view of the manufacturing and assembly process of the 2-ducts 1-stroma biochip with different stroma inlets, according to an example of the present disclosure.

FIG. 6A illustrates an isometric view of a 2-duct 1-stroma biochip with multiple stroma per duct, according to an example of the present disclosure.

FIG. 6B illustrates an exploded view of a 2-duct 1-stroma biochip with multiple stroma per duct, according to an example of the present disclosure.

FIG. 6C illustrates an isometric view of the manufacturing and assembly process of the 2-ducts 1-stroma biochip with multiple stroma per duct, according to an example of the present disclosure.

FIG. 7A illustrates a top view of an embodiment of a human-on-biochip plate, according to an example of the present disclosure.

FIG. 7B illustrates a top view of an individual biochip component of the human-on-chip plate, according to an example of the present disclosure.

FIG. 7C illustrates an exploded view of a human-on-chip plate, according to an example of the present disclosure.

FIG. 8A illustrates a top view of an embodiment of a human-on-chip where individual biochip entities are incorporated as one part within a chassis, and a valving mechanism connecting individual biochip components comprises a cylindrical rotational mechanism, according to an example of the present disclosure.

FIG. 8B illustrates a top view of an entity of an embodiment of a human-on-chip where individual biochip entities are incorporated as one part within a chassis and a valving mechanism connecting individual biochip components comprises a cylindrical rotational mechanism, according to an example of the present disclosure.

FIG. 8C illustrates an exploded view of an embodiment of a human-on-chip where individual biochip entities are incorporated as one part within a chassis, and a valving mechanism connecting individual biochip components comprises a cylindrical rotational mechanism, according to an example of the present disclosure.

FIG. 9A illustrates an isometric view of an embodiment of a human-on-chip where individual biochip entities are incorporated as one part within a chassis, and a valving mechanism connecting individual biochip components comprises a flexible membrane mechanism actuated pneumatically (isometric view), according to an example of the present disclosure.

FIG. 9B illustrates a top view of another embodiment of a human-on-chip where individual biochip entities are incorporated as one part within a chassis, and a valving mechanism connecting individual biochip components comprises a flexible membrane mechanism actuated pneumatically (top view), according to an example of the present disclosure.

FIG. 9C illustrates a top view of another embodiment of a human-on-chip where individual biochip entities are incorporated as one part within a chassis, and a valving mechanism connecting individual biochip components comprises a flexible membrane mechanism actuated pneumatically (top view), according to an example of the present disclosure.

FIG. 9D illustrates a bottom view of an embodiment of lower chassis features of another embodiment of a human-on-chip where individual biochip entities are incorporated as one part within a chassis and a valving mechanism connecting individual biochip components comprises a flexible membrane mechanism actuated pneumatically (top view), according to an example of the present disclosure.

FIG. 10A illustrates a bottom view of an embodiment of a HOC plate where OOCs are removable from the HOC plate, and fluid is accessible from a side, according to an example of the present disclosure.

FIG. 10B illustrates a top view of an embodiment of a HOC heating plate where OOCs are removable from the HOC plate, and fluid is accessible from a side, according to an example of the present disclosure

FIG. 10C illustrates a dimetric view of an embodiment of an in-plate channeling of an embodiment of a HOC plate where OOCs are removable from the HOC plate, and fluid is accessible from a side, according to an example of the present disclosure.

FIG. 10D illustrates an exploded view of an embodiment of a HOC plate where OOCs are removable from the HOC plate, and fluid is accessible from a side, according to an example of the present disclosure.

FIG. 11 illustrates a top view of an embodiment of a HOC plate where OOCs are removable from the HOC plate, and fluid and cells are sampled on-the-spot, according to an example of the present disclosure.

FIG. 12 illustrates an isometric view of an embodiment of a HOC plate where OOCs are removable from the HOC plate, and fluid and cells are sampled on-the-spot through at least one of channels, chambers, and valves, according to an example of the present disclosure.

FIG. 13 illustrates a schematic view of an embodiment of in-plate channeling of an embodiment of a HOC plate where two duct biochips are connected to a one duct biochip, according to an example of the present disclosure.

FIG. 14 illustrates a schematic diagram of an embodiment of the present disclosure comprising pressure sensors at each of a plurality of biochip inlet and outlet fluid ports to manipulate the pressure inside the biochips' micro-channels, allowing fluid to be controllably perfused from the ductal channel to the stromal one or vice versa, according to an example of the present disclosure.

FIG. 15A illustrates a perspective view of a hydraulic valving mechanism, connecting individual biochips, according to an example of the present disclosure.

FIG. 15B illustrates a perspective view of a cylindrical roll valving mechanism, connecting individual biochips, according to an example of the present disclosure.

FIG. 15C illustrates a perspective view of a flexible membrane valving mechanism connecting individual biochips, according to an example of the present disclosure.

FIG. 15D illustrates an exploded view of a flexible membrane valving mechanism connecting individual biochips, according to an example of the present disclosure.

FIG. 15E illustrates a perspective view of a biochip connection valve assembly process, according to an example of the present disclosure.

FIG. 16A illustrates a perspective view of a directional change flow valve plate actuator, according to an example of the present disclosure.

FIG. 16B illustrates a detailed view of a directional change flow valve plate actuator, according to an example of the present disclosure.

FIG. 16C illustrates a perspective view of a solenoidal magnet open-close valve, according to an example of the present disclosure.

FIG. 16D illustrates a detailed view of a solenoidal magnet open-close valve, according to an example of the present disclosure.

FIG. 16E illustrates a perspective view of a solenoidal magnet open-close valve plate, according to an example of the present disclosure.

FIG. 16F illustrates a detailed view of a solenoidal magnet inlet-outlet valve actuator plate, according to an example of the present disclosure.

FIG. 17A illustrates a perspective view of a pneumatic actuated open-close membrane valve that may be normally closed, according to an example of the present disclosure.

FIG. 17B illustrates an exploded view of a pneumatically actuated open-close membrane valve that may be normally closed, according to an example of the present disclosure.

FIG. 17C illustrates a perspective view of another embodiment of a pneumatically actuated open-close membrane valve that may be normally opened, according to an example of the present disclosure.

FIG. 17D illustrates an exploded view of another embodiment of a pneumatic actuated open-close membrane valve that is normally opened, according to an example of the present disclosure.

FIG. 18A illustrates a perspective view of a bistable beam pneumatically actuated on a flexible tube valve, according to an example of the present disclosure.

FIG. 18B illustrates an exploded view of a bistable beam pneumatically actuated on a flexible tube valve, according to an example of the present disclosure.

FIG. 19A illustrates a perspective view of a bistable magnetic drilled-piston valve, according to an example of the present disclosure.

FIG. 19B illustrates an exploded view of an embodiment of a bistable magnetic drilled-piston valve, according to an example of the present disclosure.

FIG. 20A illustrates a perspective view of a bistable mechanically actuated flexible tube valve, according to an example of the present disclosure.

FIG. 20B illustrates a top view of an embodiment of a bistable mechanically actuated flexible tube valve in which one bistable beam is in a closed position, according to an example of the present disclosure.

FIG. 20C illustrates a cross-sectional view of a bistable mechanically actuated flexible tube valve in an open position, according to an example of the present disclosure.

FIG. 20D illustrates a cross-sectional view of a bistable mechanically actuated flexible tube valve in a closed position, according to an example of the present disclosure.

FIG. 21A illustrates a perspective view of a mechanically actuated ball open-close valve, according to an example of the present disclosure.

FIG. 21B illustrates a close-up perspective view of an embodiment of a mechanically actuated ball open-close valve (wherein a biochip and a screwdriver are moving), according to an example of the present disclosure.

FIG. 21C illustrates a detailed view of a socket engaging with a valve to switch it into an open or closed position, according to an example of the present disclosure.

FIG. 21D illustrates a cross-section of a detailed view of a mechanically actuated ball open-close valve, according to an example of the present disclosure.

FIG. 22A illustrates a cross-section view of a permanently sealed open-close valve in a closed position, according to an example of the present disclosure.

FIG. 22B illustrates a side view of a permanently sealed open-close valve in a pre-set closed position where a pin is tightly inserted into a channel, blocking it, according to an example of the present disclosure.

FIG. 22C illustrates a cross-section view of a permanently sealed open-close valve in a pre-set open position, according to an example of the present disclosure.

FIG. 22D illustrates a side view of a permanently sealed open-close valve in an open position where a pin is not tightly inserted into a channel, according to an example of the present disclosure.

FIG. 23A illustrates a perspective view of an embodiment of an assembled operating system, according to an example of the present disclosure.

FIG. 23B illustrates a perspective view of an embodiment of an assembled operating system's main components, according to an example of the present disclosure.

FIG. 23C illustrates a top perspective view of an embodiment of an assembled operating system's fluidic components, according to an example of the present disclosure.

FIG. 23D illustrates a perspective view of an embodiment of an assembled operating system with a mixing chamber drawer open, according to an example of the present disclosure.

FIG. 23E illustrates an exploded view of an embodiment of an operating system, according to an example of the present disclosure.

FIG. 23F illustrates a side view of an embodiment of an assembled operating system comprising pressure-driven pumping for a plurality of microchambers, according to an example of the present disclosure.

FIG. 23G illustrates a bottom perspective view of an embodiment of an assembled operating system comprising pressure-driven pumping for a plurality of microchambers, according to an example of the present disclosure.

FIG. 23H illustrates a top perspective view of an embodiment of an assembled operating system comprising pressure-driven pumping for a plurality of microchambers, according to an example of the present disclosure.

FIG. 23I illustrates a top perspective view of an embodiment of an assembled operating system comprising pressure-driven pumping for a plurality of microchambers, according to an example of the present disclosure.

FIG. 23J illustrates an exploded view of an embodiment of an assembled operating system comprising pressure-driven pumping for a plurality of microchambers, according to an example of the present disclosure.

FIG. 23K illustrates a schematic view of an embodiment of an assembled operating system flow diagram, according to an example of the present disclosure.

FIG. 23L illustrates a schematic of a 1-to-72 distributor that connects a mixing chamber with a plurality of media chambers connected to a human-on-chip plate, according to an example of the present disclosure.

FIG. 24 illustrates a schematic of a detailed embodiment of an assembled operating system, according to an example of the present disclosure.

FIG. 25A illustrates a perspective view of another embodiment of an operating system cover, according to an example of the present disclosure.

FIG. 25B illustrates an exploded view of an operating system cover, according to an example of the present disclosure.

FIG. 25C illustrates a perspective view of a human-chip insertion process inside an operating system, according to an example of the present disclosure.

FIG. 25D illustrates a perspective view of a spectroscopy probe and sensing plates insertion into an operating system, according to an example of the present disclosure.

FIG. 25E illustrates an exploded view of operating system components, according to an example of the present disclosure.

FIG. 25F illustrates a perspective view of a fluidic control part of an operating system, according to an example of the present disclosure.

FIG. 26A illustrates a perspective view of another embodiment of an operating system, according to an example of the present disclosure.

FIG. 26B illustrates an exploded view of another embodiment of an operating system, according to an example of the present disclosure.

FIG. 26C illustrates a top view of another embodiment of an operating system, according to an example of the present disclosure.

FIG. 27A illustrates an isometric view of an embodiment of an assembly of media chambers of another embodiment of a human-on-chip where individual biochip entities are incorporated as one part within a chassis, and a valving mechanism connecting individual biochip components is a flexible membrane mechanism actuated pneumatically.

FIG. 27B illustrates an isometric exploded assembly of media chambers of another embodiment of a human-on-chip where individual biochip entities are incorporated as one part within a chassis and a valving mechanism connecting individual biochip components is a flexible membrane mechanism actuated pneumatically.

FIG. 27C illustrates a perspective view of a micro media chamber plate, according to an example of the present disclosure.

FIG. 27D illustrates an exploded view of a micro media chamber plate, according to an example of the present disclosure.

FIG. 27E illustrates a perspective view of a piston plate, according to an example of the present disclosure.

FIG. 27F illustrates a perspective view of another embodiment of a piston plate, according to an example of the present disclosure.

FIG. 27G illustrates a side view of an embodiment of a piston plate, according to an example of the present disclosure.

FIG. 27H illustrates an exploded detailed view of a piston of a piston plate of FIGS. 27E-27G, according to an example of the present disclosure.

FIG. 27I illustrates an isometric perspective view of a piston actuator plate, according to an example of the present disclosure.

FIG. 27J illustrates an isometric perspective view of a piston actuator plate, according to an example of the present disclosure.

FIG. 27K illustrates a top-perspective view of a piston actuator plate, according to an example of the present disclosure.

FIG. 28A illustrates a schematic of processes of a human-on-chip operating system, according to an example of the present disclosure.

FIG. 28B illustrates a schematic view of a media chamber filling process of a human-on-chip operating system, according to an example of the present disclosure.

FIG. 28C illustrates a schematic view of a media chamber emptying process of a human-on-chip operating system, according to an example of the present disclosure.

FIG. 28D illustrates a schematic view of a media chamber seeding process of a human-on-chip operating system, according to an example of the present disclosure.

FIG. 28E illustrates a schematic view of a media sample-collecting process of a human-on-chip operating system, according to an example of the present disclosure.

FIG. 28F illustrates a schematic view of a cell sample-collecting process of a human-on-chip operating system, according to an example of the present disclosure.

FIG. 28G illustrates a schematic view of a cell sample-collecting process of a human-on-chip operating system, according to an example of the present disclosure.

FIG. 28H illustrates a schematic view of a cell sample-collecting process of a human-on-chip operating system, according to an example of the present disclosure.

FIG. 28I illustrates a schematic view of a cell sample-collecting process of a human-on-chip operating system, according to an example of the present disclosure.

FIG. 29A illustrates a flow diagram of a fluid control in a triple measurement mechanism, according to an example of the present disclosure.

FIG. 29B illustrates a logic control diagram of a fluid control for FTIR sampling, according to an example of the present disclosure.

FIG. 30A illustrates a schematic view to pump a sample from a media chamber to a biochip, according to an example of the present disclosure.

FIG. 30B illustrates a schematic view to pump a sample from a biochip to a measurement device, according to an example of the present disclosure.

FIG. 31A illustrates a schematic view of pumping gel into a stroma channel, according to an example of the present disclosure.

FIG. 31B illustrates a schematic view of pumping gel out of a stroma channel, according to an example of the present disclosure.

FIG. 32A illustrates an isometric of a fully assembled embodiment of a mixing chamber, according to an example of the present disclosure.

FIG. 32B illustrates an isometric view of an embodiment of a cell sourcing mixing chamber, according to an example of the present disclosure.

FIG. 32C illustrates a top view of an embodiment of a media sourcing mixing chamber, according to an example of the present disclosure.

FIG. 32D illustrates an isometric view of a cell chamber and media chamber side by side, according to an example of the present disclosure.

FIG. 32E illustrates a schematic view of a pressure and gas container, according to an example of the present disclosure.

FIG. 32F illustrates a cross sectional view of a media chamber used in a mixing chamber, according to an example of the present disclosure.

FIG. 32G illustrates a schematic view of an embodiment of a bubble filter and pressure sensors of a mixing chamber, according to an example of the present disclosure.

FIG. 33A illustrates a schematic view of an embodiment of a mixing chamber where all 36 tubes leave the mixing system, according to an example of the present disclosure.

FIG. 33B illustrates a perspective view of an embodiment of a mixing chamber where all 36 tubes feed into only 3 tubes leaving a mixing system, according to an example of the present disclosure.

FIG. 33C illustrates a schematic view of a media chamber used in a mixing chamber, according to an example of the present disclosure.

FIG. 33D illustrates a schematic view of an embodiment of a mixing chamber where three pressure valves are connected to cell chambers, according to an example of the present disclosure.

FIG. 33E illustrates a schematic view of an embodiment of a mixing chamber where each row of cell chambers is connected to a 1/12-way valve that can switch between them and send a sample through at least one of a flow sensor, air filter, and a pressure gauge connected to a quick connect that may link the mixing chamber with a human-on-chip plate, according to an example of the present disclosure.

FIG. 33F illustrates a schematic view of a single-cell chamber entity wherein the single-cell chamber entity is connected to pressure and gas from one side and to a 1/12-way valve from another side, which interim is connected to a sensor, air filter, and a pressure gauge connected to a quick connect which links the mixing chamber with a human-on-chip plate, according to an example of the present disclosure.

FIG. 33G illustrates a schematic view of an embodiment of a mixing chamber comprising three pressure tubes connected to a media chamber, according to an example of the present disclosure.

FIG. 33H illustrates a schematic view of an embodiment of a mixing chamber comprising 12 gas tubes connected to a media chamber, according to an example of the present disclosure.

FIG. 33I illustrates a schematic view of an embodiment of a mixing chamber comprising 3 pressure valves connected to a plurality of media chambers, according to an example of the present disclosure.

FIG. 33J illustrates a schematic view of an embodiment of a mixing chamber where each of a plurality of rows of a plurality of media chambers is connected to a 1/4-way valve configured to switch between the plurality of rows and send a sample through a flow sensor, air filter, and a pressure gauge connected to a quick connect that links the mixing chamber with a human-on-chip plate, according to an example of the present disclosure.

FIG. 34A illustrates a schematic view of an embodiment of a mixing chamber where each of a plurality of cell chambers have access to O2, CO2, and N2 bottles and O2 and CO2 sensors that are located under the cell chambers, according to an example of the present disclosure.

FIG. 34B illustrates a schematic view of an embodiment of a mixing chamber where each of a plurality of media chambers have access to O2, CO2, and N2 bottles and O2 and CO2 sensors that are located under the media chambers, according to an example of the present disclosure.

FIG. 34C illustrates a schematic view of an embodiment of O2 and CO2 sensors at the level of tissues grown in biochips, according to an example of the present disclosure.

FIG. 35A illustrates an exploded view of a heat exchange plate passing temperature-controlled fluid in thermal contact with a human-on-chip, according to an example of the present disclosure.

FIG. 35B illustrates a isometric view of an exchange generator chamber that heats and/or cools fluid coming to a heat exchange plate, according to an example of the present disclosure.

FIG. 36A illustrates a schematic view of an embodiment of a machine starting mechanism causing fluid to run into a system and further ensuring that there are no blocked channels and/or damaged valves, actuators, or sensors, according to an example of the present disclosure.

FIG. 36B illustrates a schematic view of an embodiment of a machine starting mechanism causing temperature, O2, and CO2 levels to reach a desired valve while flushing a system pre-cell seeding, actuator, or sensor damaged, according to an example of the present disclosure.

FIG. 36C illustrates a schematic view of an embodiment of a machine starting mechanism using pressure sensors with assistance from microscopy computer vision to make sure there is no air bubble in the system prior to cell seeding (including, but not limited to pressuring all the liquids, imaging, light contrast, fluorescence), according to an example of the present disclosure.

FIG. 36D illustrates a schematic view of an embodiment of a machine starting mechanism using thermal imaging (such as flash thermography) to detect and view at least one of fluid flow, un-wetted surfaces, and air bubbles, according to an example of the present disclosure.

FIG. 36E illustrates a schematic view of an embodiment of a machine starting and running mechanism showing temperature, O2, CO2, and pressure sensors in addition to fluid channeling, CO2 and O2 mixing, air bubble traps, and heating plates, controlling all the parameters of a system, according to an example of the present disclosure.

FIG. 37A illustrates a perspective view of an embodiment of a microscopy and spectroscopy mechanism comprising a plurality of lenses, according to an example of the present disclosure.

FIG. 37B illustrates a perspective view of an embodiment of a microscopy and spectroscopy mechanism comprising one lens configured to move in an x-y plane, according to an example of the present disclosure.

FIG. 37C illustrates a schematic view of an embodiment of a FTIR reading mechanism comprising a plurality of sensors, according to an example of the present disclosure.

FIG. 37D illustrates a schematic view of an embodiment of a FTIR reading mechanism comprising a single sensor, and wherein fluid is being sampled and measured with a specified time gap, according to an example of the present disclosure.

FIG. 37E illustrates a schematic view of a detailed sampling mechanism of an embodiment of a FTIR reading mechanism comprising a single sensor, and the fluid is being sampled and measured with a time a specified time gap, according to an example of the present disclosure.

FIG. 37F illustrates a schematic view of an embodiment of a FTIR reading mechanism directly from the HOC plate channels, according to an example of the present disclosure.

FIG. 38A illustrates a top view of possible biochip arrangements for human-on-chip modeling, according to an example of the present disclosure.

FIG. 38B illustrates a perspective view of possible biochip arrangements for human-on-chip modeling, according to an example of the present disclosure.

FIG. 38C illustrates a top view of another embodiment of possible biochip arrangements for human-on-chip modeling, according to an example of the present disclosure.

FIG. 38D illustrates a perspective view of another embodiment of possible biochip arrangements for human-on-chip modeling, according to an example of the present disclosure.

FIG. 39A illustrates a cross-sectional view of an embodiment of a tubular architecture of a tissue in 2-duct biochips, according to an example of the present disclosure.

FIG. 39B illustrates a perspective view of an embodiment of multiple cultured tissues in biochips connected with a common duct/vessel, according to an example of the present disclosure.

FIG. 39C illustrates a cross-sectional view of an embodiment of an organ-on-chip grown in a human-on-chip, according to an example of the present disclosure.

FIG. 39D illustrates a cross-sectional view of an embodiment of a Liquid-air-Interface Lung-on-chip grown in a human-on-chip, according to an example of the present disclosure.

FIG. 39E illustrates a cross-sectional view of an embodiment of a glioblastoma model and a blood-brain barrier in a biochip, according to an example of the present disclosure.

### DETAILED DESCRIPTION OF EXAMPLE EMBODIMENTS

The present disclosure provides five aspects that enable a system of precise and repeatable semi-automatic predictive experimentations. The provided experimentation enables the accurate creation and maintenance of tissue models, drug testing, real-time sampling and measurement, in addition to software-assisted control and data analysis. The provided system enables more precise prediction of a drug's toxicity, efficacy, and dosage. This system is a powerful tool for personalized and precise drug prognosis.

A first aspect of the present disclosure provides a hybrid human-on-chip tissue culture platform for a human model, wherein the hybrid human-on-chip tissue culture platform can be used by itself and/or with the aid of the operating system. This platform has a unique modularity and automation ability as it can control multiple OOCs, which can be isolated or connected to each other in customizable permutations that can be changed during an experiment. The platform can supply each of the connected OOCs with similar or different fluid components, flow rates, pressures, or shear forces, allowing for an accurate mimicry of human body fluid dynamics.

A second aspect of the present disclosure provides a biomimetic tissue culture platform that enables the fundamental aspects of a biomimetic model. The provided biochip embeds a number of cylindrical porous ductal scaffolds forming different compartments in the biochip.

A third aspect of the present disclosure provides a semi- or fully-automated fluidic control system of valving and pumping, wherein relatively very small amounts of volumes could be manipulated and controlled in any way, moving samples across all of the regions of the system and also into and out of the system without diluting the sample.

A fourth aspect of the present disclosure provides a semi-automated sample collection and real-time measurement add-ons, wherein the configuration of the system allows for interface with microscopy, spectroscopy, Fourier Transform Infrared (FTIR), and other measurement devices to measure every sample while the experiments are ongoing and pumping into and out of the system.

A fifth aspect of the present disclosure provides software to automate and control the operating system, wherein the software contains different commands for different experimental setups. Additionally, the software may be in communication with the measurement devices and allow data acquisition. The software may analyze the data and control the system accordingly.

A sixth aspect of the present disclosure provides a multiplex logic microvalve system, wherein a plurality of bistable valves can be actuated through a reduced number of actuating inputs.

### Multi-Duct Biochip

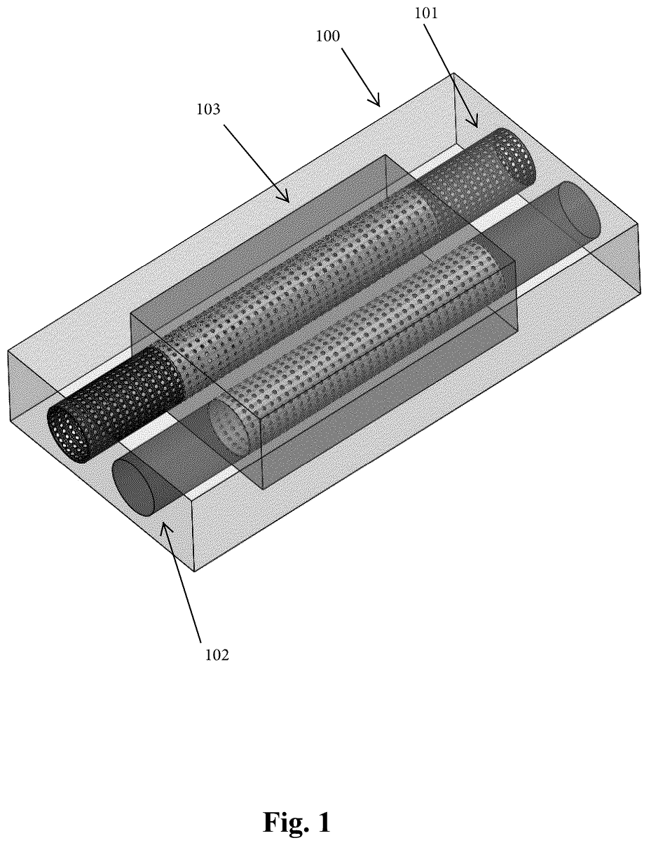

Turning to FIG. 1, the biochip 100 contains two ducts and one stroma, the ducts being formed from ultrathin porous membranes that are cylindrical, which may or may not contain a curve. In an additional embodiment, the ducts could be made of gore flexible tubing or any suitable material or combination of materials, and the pores may be created by laser gunning or by extrusion. In an embodiment, the biochip 100 may contain multiple ducts 101, 102 and at least one stroma 103. A solution may be passed through the ducts 101, 102 and/or inserted into the at least one stroma 103. Further, a solution different from the solution being passed through the plurality of ducts 101, 102 may be inserted in the stroma 103. In an additional embodiment, known materials such as stents may be utilized in the biochips 100.

In an embodiment, separate membranes that are hydrophobic and hydrophilic could be utilized to create multiple ducts. In an additional embodiment, one of the multiple ducts could be formed from a single membrane with different regions treated to be hydrophobic or hydrophilic by plasma or UV treatment or through coating. Depending on the specific requirements of the user of the system, the hydrophobic and hydrophilic properties of the multiple ducts may be modulated according to known treatment techniques.

In an embodiment, the biochip 100 may contain a plurality of cavities to allow for multiple organs to be cultured on a single multi-duct biochip.

In an embodiment, biochips with a single duct may also be utilized by the presently disclosed system. For example, in an embodiment, the biochip utilized may be the biochip described in PCT App. No. PCT/QA2021/050016, filed Jun. 25, 2021, entitled DUCT ORGANOID-ON-CHIP, assigned to the assignee of the present invention and hereby incorporated by reference in its entirety, which describes a biomimetic tissue culture platform or biochip that enables the fundamental aspects of a biomimetic model.

FIG. 1 illustrates a schematic view of a biochip 100 of two ducts 101 and 102 surrounded by one stroma 103, according to an example of the present disclosure.

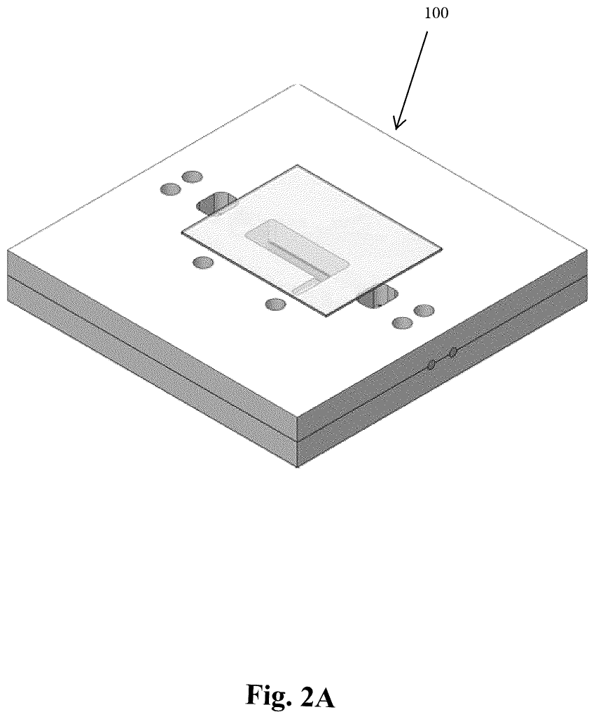

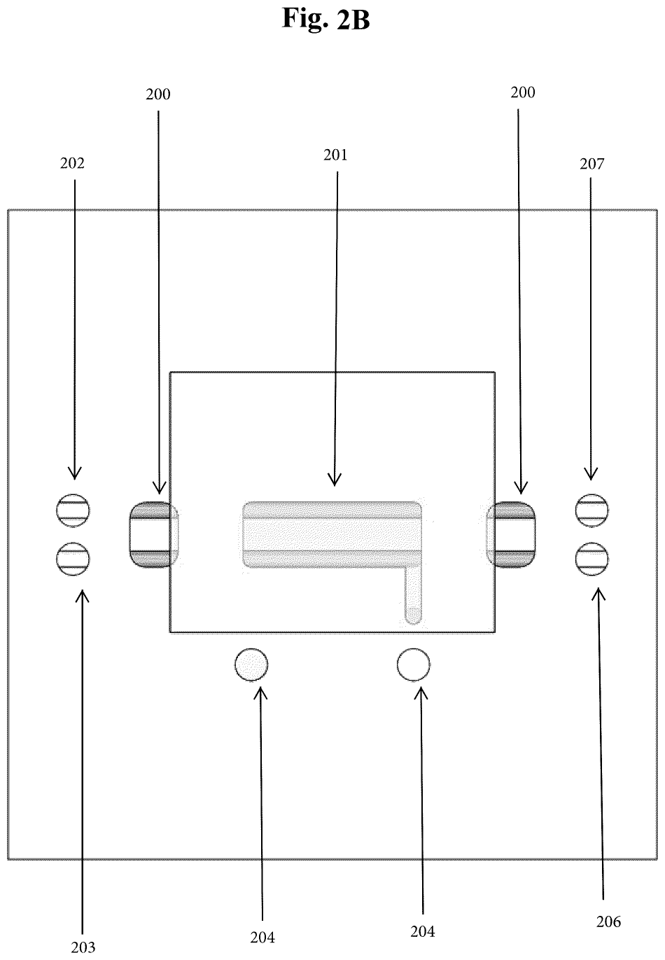

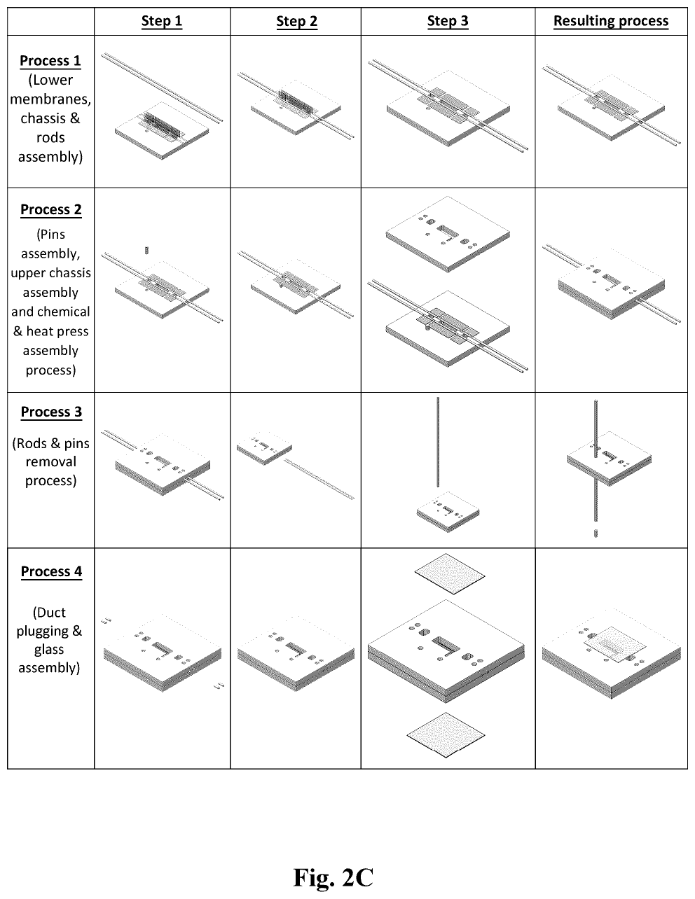

FIG. 2A illustrates a perspective view of a 2-duct, 1-stroma biochip 100, according to an example of the present disclosure. In an embodiment, a chassis of the biochip 100 is constructed of a material that does not obstruct optical or spectroscopic access to the multiple ducts.

FIG. 2B illustrates a top view of another embodiment of a 2-duct 101, 102, 1-stroma 103 biochip 100, according to an example of the present disclosure. In an embodiment, the biochip 100 may include two porous cylindrical ductal channels surrounded by the same stroma chamber 201 and air filtering areas 200 for each duct which may allow for air bubbles to be released out of the biochip 100. In some examples, the air filtering area 200 may also allow air to enter the biochip which allows the cells being cultured to have access to gas. In some examples, the biochip 100 may include on each duct an inlet 202, 203 and outlet 206, 207, and for the stroma channel an inlet/outlet 204. The biochip 100 may also include multiple segments of duct surrounded by plastic.

FIG. 2C shows an illustration of the manufacturing and assembly process of the 2-ducts 1-stroma biochip 100, according to an example of the present disclosure. In an example, the biochip 100 configured according to this method may include an air filtering area 200, a duct 100 surrounded by stroma 103, a duct inlet 202-203, a duct outlet 207-206, a stroma channel inlet, and a stroma channel outlet 204. This figure illustrates the steps and processes, according to this method, to execute the bonding process of the biochip 100 components where the ductal scaffold is curved, tensioned, and bound to the chassis at its extremities in one step. For example, the method includes the on-chassis locating and the curving process of the hydrophobic and hydrophilic membranes over a rod and bonding it at its extremities to the chassis using glue, heat, or chemicals. After locating the membranes on the chassis, they are curved over the two rods that will form each of the two ducts, tension is applied on the membranes, the extremities are bound to the chassis, and the pin is assembled into the outlet stroma hole of the lower chassis. Then, all components are sandwiched between the two chassis and then bound using chemical-aided heat-press bonding. Finally, the method may involve a removal process of the rods and the pin, plugging the extremities of both ductal channels beyond the inlet and outlet holes, and assembling coverslip glasses on both surfaces of the biochip 100.

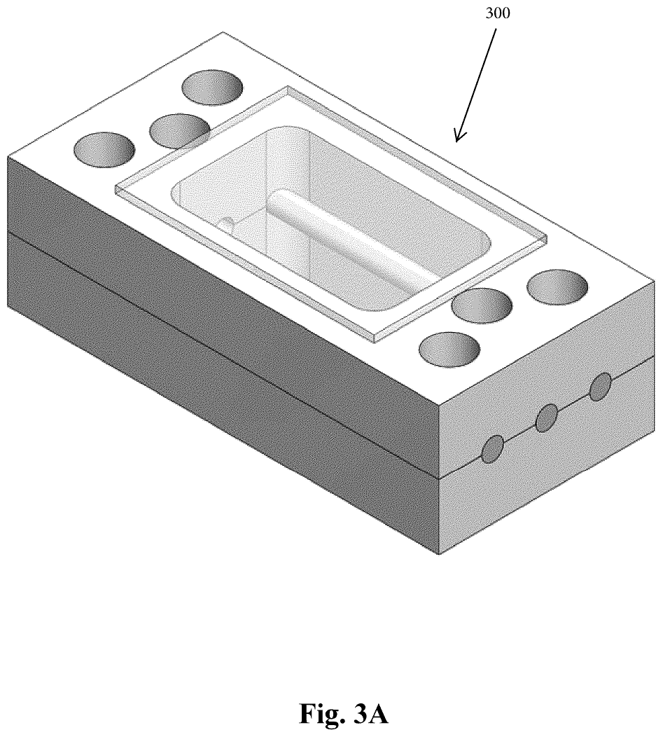

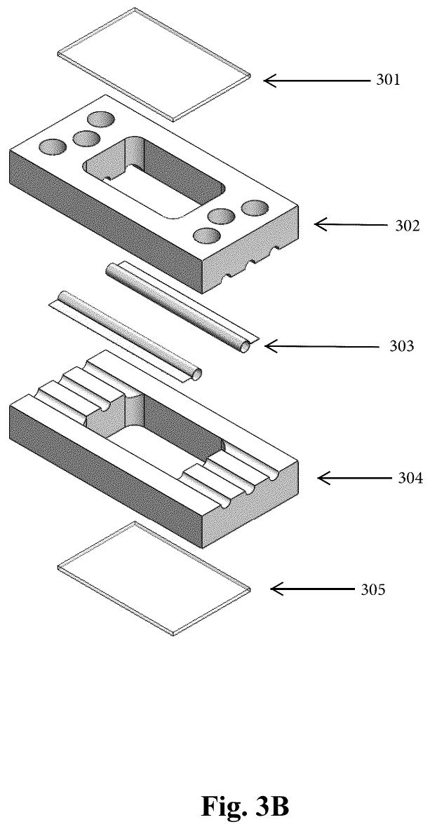

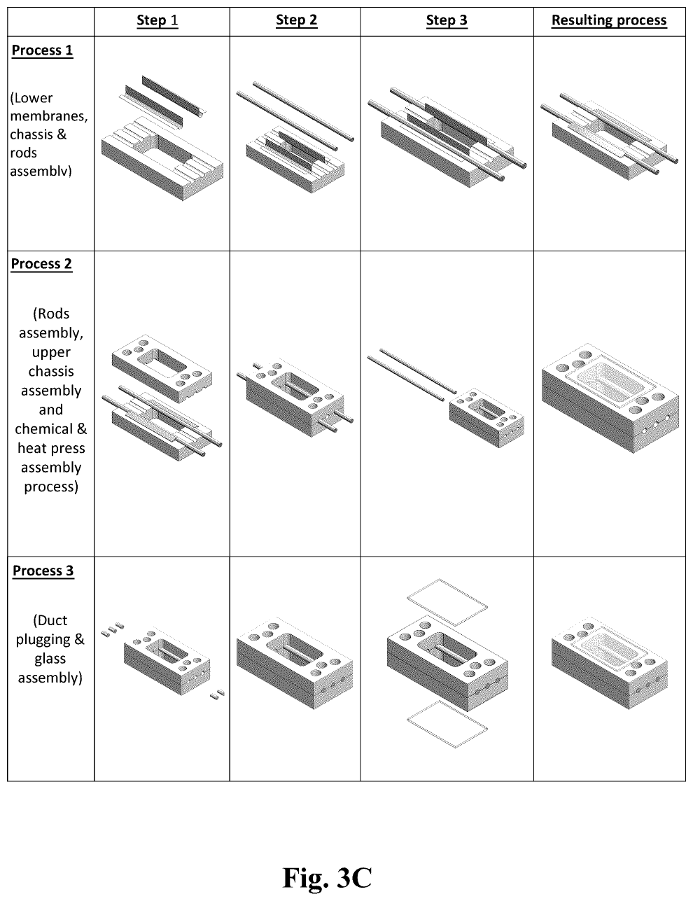

FIG. 3A illustrates another perspective view of a 2-duct, 1-stroma biochip 300, with three parallel channels, according to an example of the present disclosure.

FIG. 3B illustrates an exploded view of a 2-duct, 1-stroma biochip 300, with three parallel channels, according to an example of the present disclosure. In an embodiment, the biochip 300 consists of a lower and upper chassis 304 and 302, two porous cylindrical ductal scaffolds channels 303 that are surrounded by one stroma and covered with a bottom glass cover 305 and an upper glass cover 301. In an embodiment, two ducts with porous membranes 303 are surrounded by one stroma, in which the upper middle ducts are connected to the stroma chamber, wherein these two holes can be used for stroma feeding. In an additional embodiment, two types of epithelial or endothelial cells can be cultured in the ducts, and at least one of stromal, endothelial, or cancerous cells can be cultured in a stroma.

In an example, the biochip 300 includes a cover glass 301 on the uppermost portion of the biochip to cover at least part of the top surface of the upper chassis 302 of the biochip 100. The upper cover glass 301 may be a thin coverslip glass made of a material that is transparent and has low autofluorescence, such as, but not limited to, glass or polymer. The upper chassis 302 and the lower chassis 304 contain features such that when placed in direct contact with each other, internal space is formed to accommodate porous ductal scaffolds made of the membranes. These features may be the result of engraving in the inner layers of the chassis. In another example, the upper chassis 302 and the lower chassis 304 contain features forming the microfluidic channels leading to the external compartment of the ductal scaffolds and could be engraved on the outer surfaces of the chassis 302, 304 and covered with another chassis part or a thin coverslip creating the full channel on the lower chassis 304 to accommodate the multiple porous membrane structures.

In an embodiment, the upper chassis 302 and the lower chassis 304 contain features that hold the cylindrical ductal scaffolds 303 in position, giving access to the internal and external compartments of each of the ductal scaffolds 303. The chassis 302, 304 forming the microfluidic channels leading to the internal compartment of the ductal scaffold 303 could extend beyond the inlet and outlet holes of the channel and later be plugged using plugs post-assembly of the biochip 300. A bottom cover glass 305, similar to the upper cover glass 301, may be provided to cover the bottom-most portion of the lower chassis 304. In an example, coverslip glass forming the top & bottom layers of the biochip 300 is bonded to the chassis 302, 304 using a glass-polymeric glue. In an example, the porous membrane structure is formed by curving porous membranes into a 180° rotation, forming a cylindrical structure and bonding the excess material of the membranes that were not curved into the cylindrical structure 303. For example, two pre-bonded cylindrical hydrophobic ultrathin porous membranes on each duct can be used for filtering. These components may be combined to form multiple porous membrane ductal structures surrounded by the same stroma.

FIG. 3C illustrates the manufacturing and assembly process of a 2-duct, 1-stroma biochip 300, with three parallel channels, according to an example of the present disclosure, where the side of the membrane is tightly bonded to the lower chassis 304, after which a rod is placed inside the ductal area over the membrane allowing the membrane to rotate around the rod in a cylindrical manner of at least 180° orientation. The upper chassis 302 and the lower chassis 304 contain three parallel channels that hold two porous cylindrical ductal scaffolds, channels 303, and one stroma inlet in position giving access to the internal and external compartments of each ductal scaffold. In an embodiment, two ducts with porous membranes 303 that are surrounded by one stroma are curved in a cylindrical manner around a rod and bonding at the extremities of the chassis using glue, heat, or chemicals and by assembling pins into the outlet stroma hole of the lower chassis 304. Then, all components are sandwiched between the two chassis 302, 304, then bound using chemical-aided heat-press bonding. Finally, the method may involve a removal process of the rods and the pin, plugging the extremities of both ductal channels beyond the inlet and outlet holes, and assembling the coverslip glasses 301, 305 on both surfaces of the biochip.

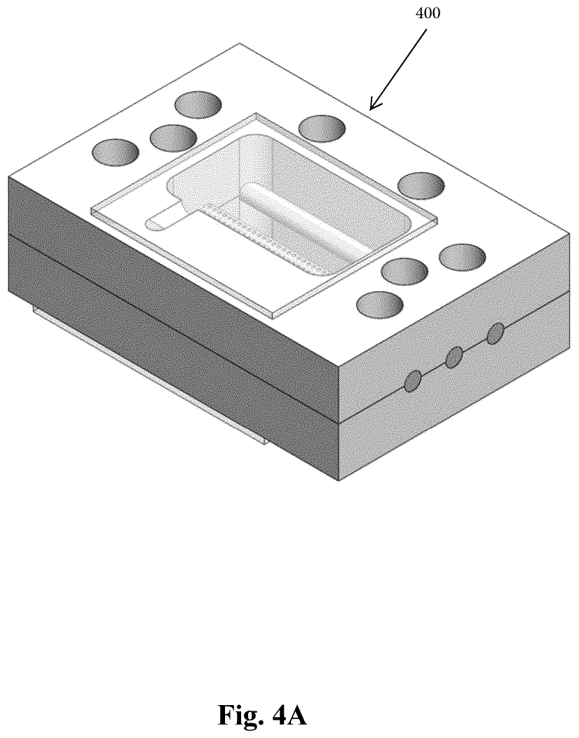

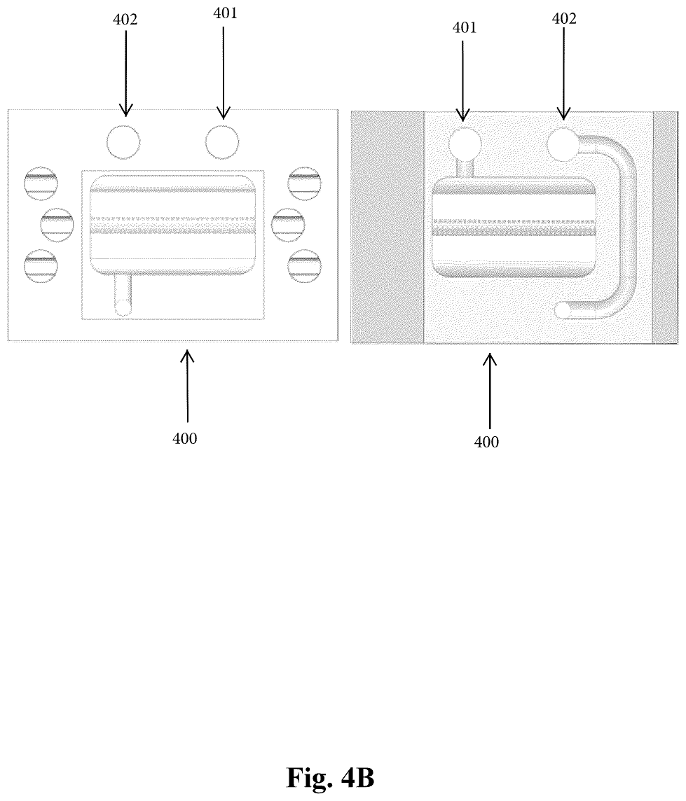

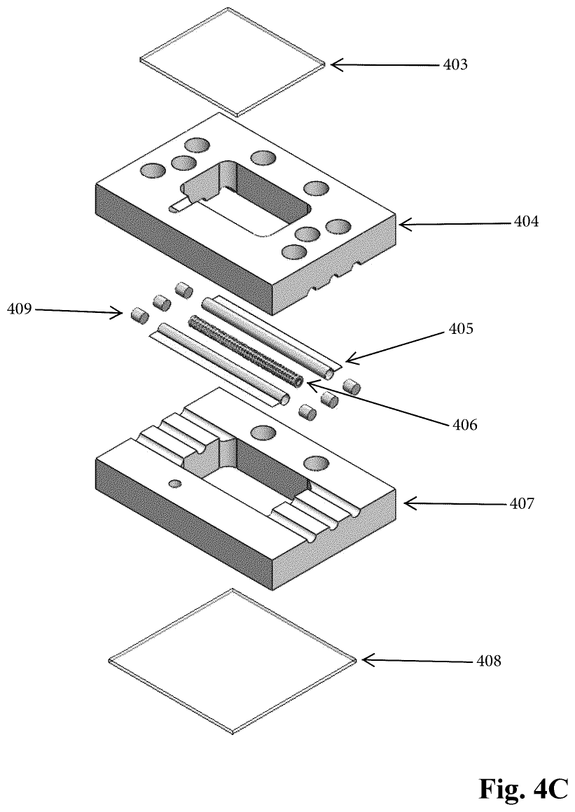

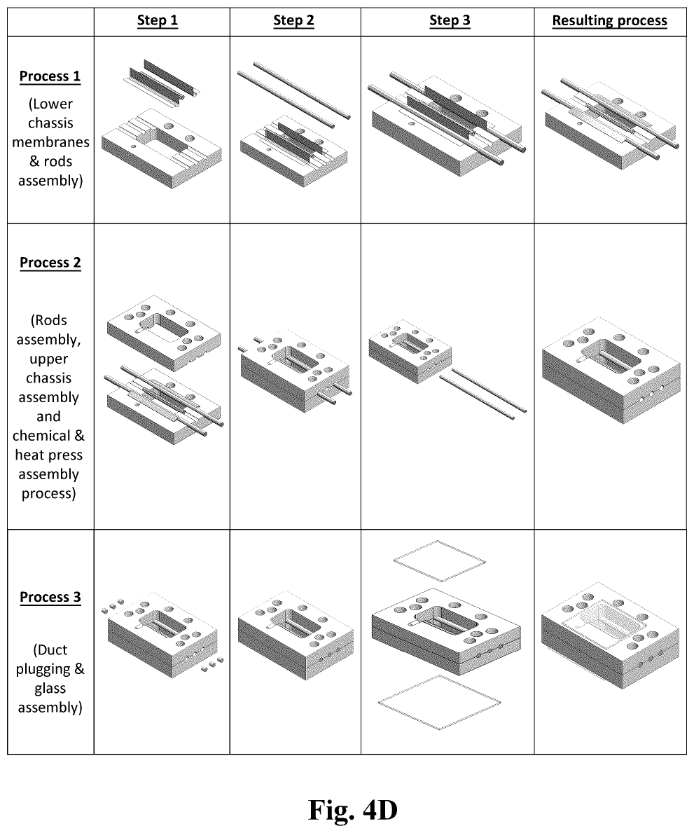

FIG. 4A illustrates a perspective view of an embodiment of a 2-duct, 1-stroma biochip 400, according to an example of the present disclosure.

FIG. 4B illustrates a top view of an additional embodiment of a 2-duct, 1-stroma biochip 400, with stroma feeding channels 401, 402 according to an example of the present disclosure.

FIG. 4C illustrates an exploded view of an additional embodiment of a 2-duct, 1-stroma biochip 400, with three parallel channels and a stroma feeding channel according to an example of the present disclosure. In an embodiment, the biochip 400 consists of a lower and upper chassis 407 and 404, two porous cylindrical ductal scaffolds channels 405, and a perforated tube 406 surrounded by the same stroma chamber having a stroma feeding channel, side pins 409, and covered with a bottom glass cover 408 and an upper glass cover 403. In an embodiment, the biochip 400 may include two porous cylindrical ductal channels 405 surrounded by the same stroma chamber having a stroma feeding channel inlet and outlet 401, 402, where these channels can be used for gel insertion into the stroma chamber. In an embodiment, the biochip 400 may include two porous cylindrical ductal scaffolds channels 405 surrounded by the same stroma chamber having a stroma feeding channel 401, 402, where the fluids insertion into the stroma is done by a perforated tube 406 that enables direct feeding to the stroma, and wide pores in the tube increase the diffusion rate between the perforated tube 406 and the stroma.

FIG. 4D illustrates the manufacturing and assembly process of a 2-duct, 1-stroma biochip 400 with three parallel channels according to an example of the present disclosure. The upper chassis 404 and the lower chassis 407 contain three parallel channels that hold two porous cylindrical ductal scaffolds channels 405 and a perforated tube 406, and one stroma inlet in position, giving access to the internal and the external compartments of each of the ductal scaffolds. In an embodiment, a perforated tube 406 is glued at its extremities in the middle channel, and the ducts are formed from ultrathin porous membranes that are curved in a cylindrical manner around a rod and bonded at its extremities to the chassis 404, 407 using glue, heat, or chemicals. The side pins 409 are assembled into the outlet stroma hole of the lower chassis 407. Then, everything is sandwiched in-between the two chassis 404, 407, then bonded using chemical-aided heat-press bonding. Finally, the method may involve a removal process for rods and pin, plugging of extremities of both ductal channels beyond the inlet and outlet holes using side pins 409, and coverslip glasses 403,408 are assembled on both surfaces of the biochip 400.

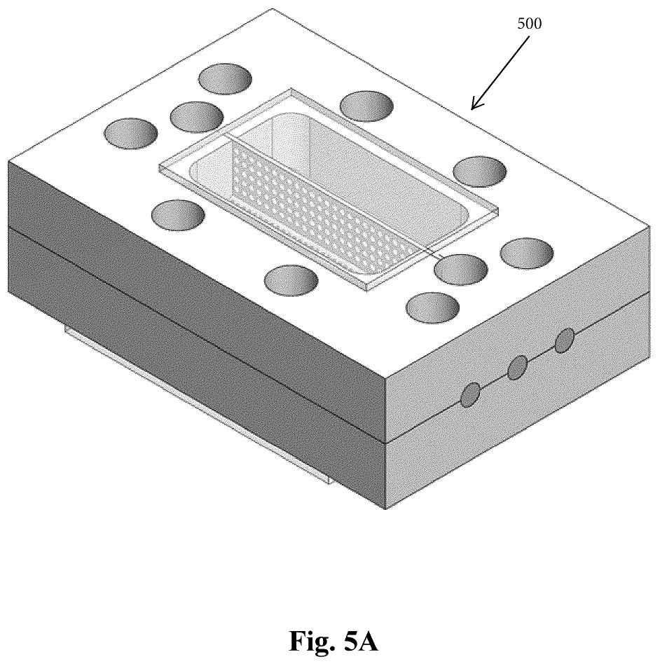

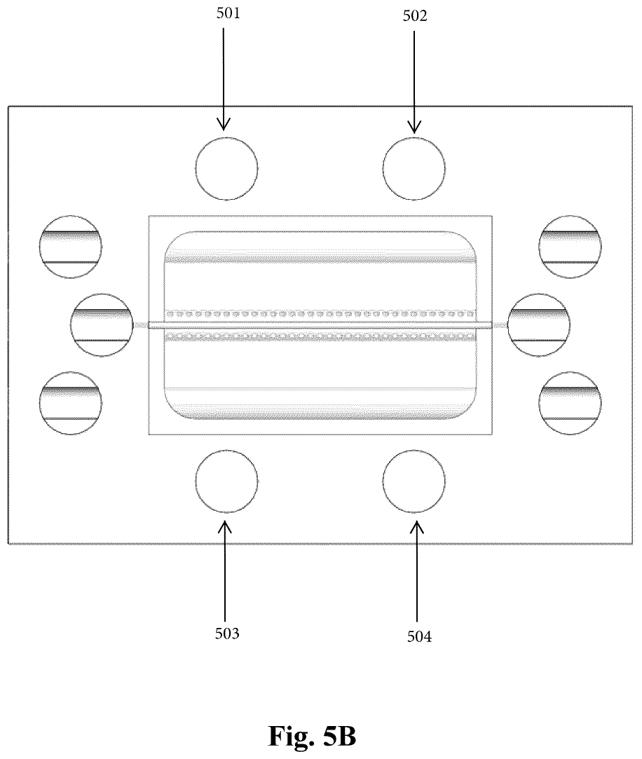

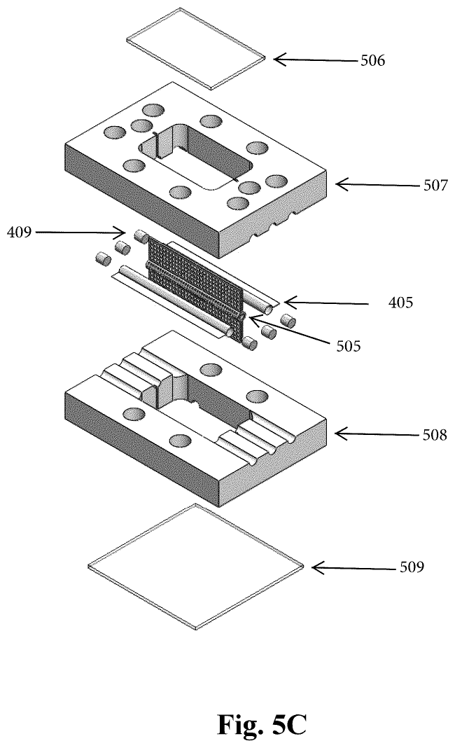

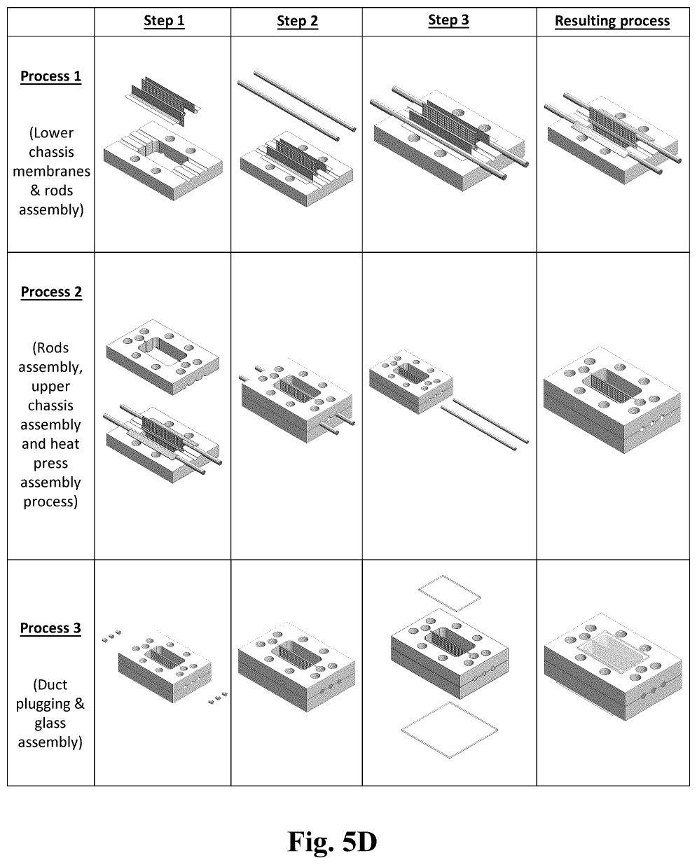

FIG. 5A illustrates a perspective view of a 2-ducts 1-stroma biochip 500 with different stroma inlets, according to an example of the present disclosure.

FIG. 5B illustrates a top view of a 2-ducts 1-stroma biochip 500 with different stroma inlets 501 and 503, and stroma outlets 502 and 504, according to an example of the present disclosure.

FIG. 5C illustrates an exploded view of a 2-ducts 1-stroma biochip 500 with different stroma inlets, according to an example of the present disclosure. In an embodiment, the biochip 500 consists of a lower and upper chassis 508, 507, two porous cylindrical ductal channels 405, and a perforated barrier 505 that splits the stroma chamber into two separate chambers of different stroma inlets 501 and 503 and outlets 502 and 504, side pins 409, and covered with a lower glass cover 509 and an upper glass cover 506. In an embodiment, the biochip 500 may include two porous cylindrical ductal channels 405 and a perforated barrier 505 that splits the stroma chamber into two separate chambers of different stroma inlets 501 and 503 and stroma outlets 502 and 504 to allow for different types of tissues to be cultured in different locations in the same stroma chamber on a 2-ducts 1-stroma biochip 500. In an embodiment, the biochip may include two porous cylindrical ductal channels 405 and a perforated barrier 505 that splits the stroma chamber into two separate chambers of different stroma inlets 501 and 503 and outlets 502 and 504, where each inlet can be used to inject the gel into a dedicated stromal area.

FIG. 5D illustrates the manufacturing and assembly process of a 2-ducts 1-stroma biochip 500 with different stroma inlets, according to an example of the present disclosure. The upper chassis 507 and the lower chassis 508 contain three parallel channels that hold two porous cylindrical ductal scaffolds 405 and two different stroma inlets for each stroma chamber in position, giving access to the internal and the external compartments of each of the ductal scaffolds 405. In an embodiment, the stroma chamber is split into two separate stromas by a perforated barrier 505 that is glued at its extremities in the middle channel. The ducts are formed from ultrathin porous membranes that are curved in a cylindrical manner around a rod, and bonded at its extremities to the chassis 507, 508 using glue, heat, or chemicals, and the side pins 409 are assembled into the outlet stroma hole of the lower chassis 508. Then, all components are sandwiched in between the two chassis, and then bonded using chemical-aided heat-press bonding. Finally, the method may involve a removal process of rods, and the pin, plugging the extremities of both ductal channels beyond the inlet and outlet holes using side pins 409, and assembling the coverslip glasses 506-509 on both surfaces of the biochip.

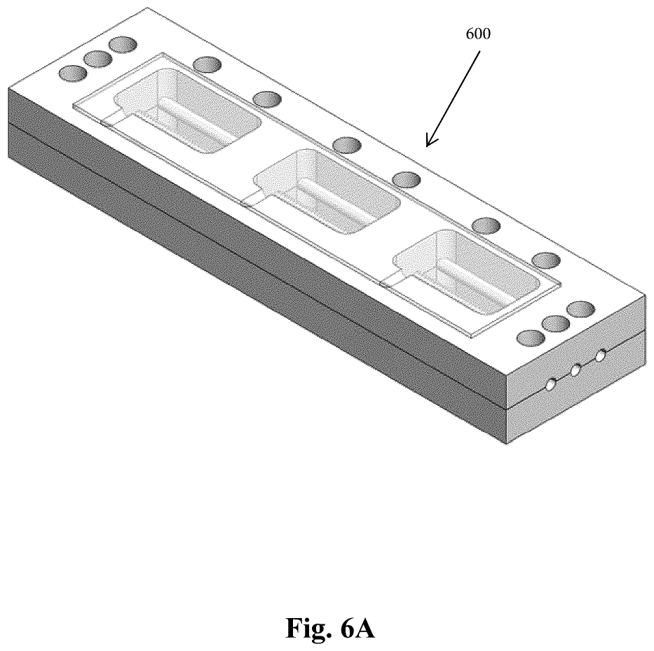

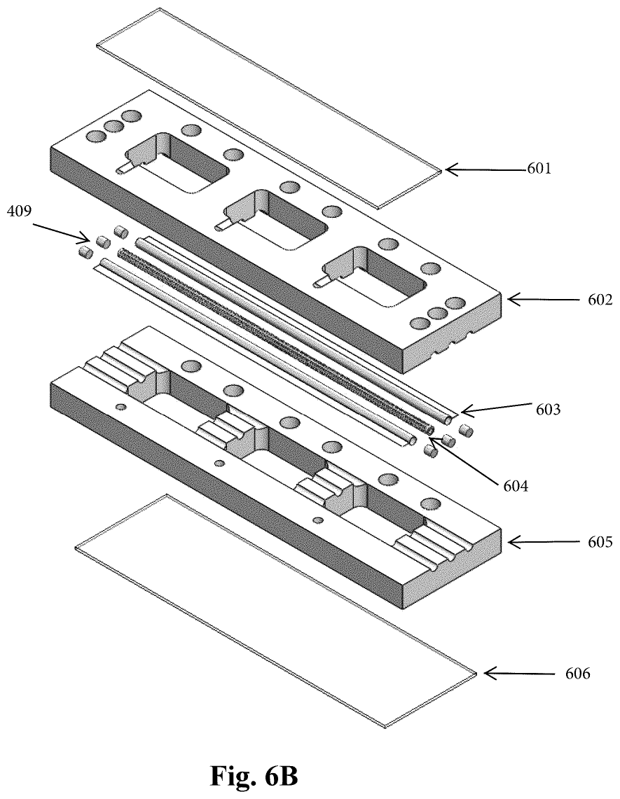

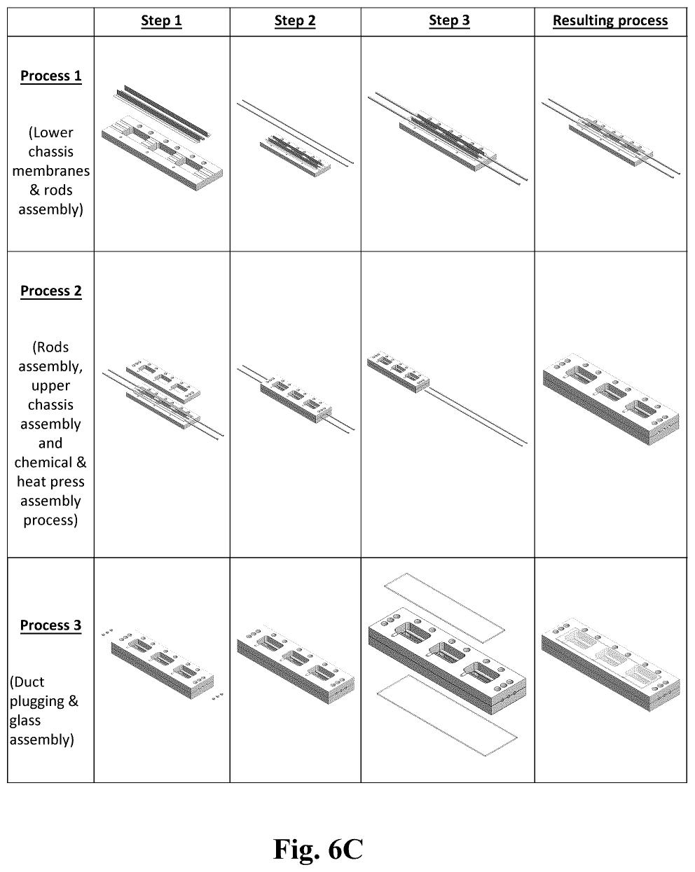

FIG. 6A illustrates a perspective view of a multiple stroma per duct biochip 600, with three stroma and two ducts, according to an example of the present disclosure. In an embodiment, the biochip 600 may contain several cavities to allow for multiple types of tissue to be cultured, by surrounding different portions of the ducts on a single multiple stroma per duct biochip 600.

FIG. 6B illustrates an exploded view of a single multiple stroma per duct biochip 600, with three stroma, and two ducts, according to an example of the present disclosure. In an embodiment, the biochip 600 consists of a lower and upper chassis 605 and 602, two porous cylindrical ductal channels 603, a perforated tube 604, side pins 409, and covered with a lower glass cover 606 and an upper glass cover 601.

FIG. 6C illustrates the manufacturing and assembly process of a multiple stroma per duct biochip 600, with three stroma and two ducts, according to an example of the present disclosure. The upper chassis 602 and the lower chassis 605 contain three parallel channels that hold two porous cylindrical ductal scaffolds 603 and one stroma inlet for each stroma chamber in position, giving access to the internal and the external compartments of each of the ductal scaffolds. In an embodiment, the ducts are formed from ultrathin porous membranes that are curved in a cylindrical manner around a rod and bonded at its extremities to the chassis using glue, heat, or chemicals, and the side pins 409 are assembled into the outlet stroma hole of the lower chassis 605. Then, all components are sandwiched in between the two chassis, then bonded using chemical-aided heat-press bonding. Finally, the method may involve a removal process for the rods and the pin, plugging the extremities of both ductal channels beyond the inlet and outlet holes, and assembling the coverslip glasses 601, 606 on both surfaces of the biochip 600.

### The Human-on-Chip Plate

In an embodiment, the human-on-chip plate contains cylindrical cross-sectional ducts surrounded by stroma so that a blood vessel duct could pass through the stroma of different tissues that also contain epithelial ducts passing through its individual stroma.

In an additional embodiment, the biochip plate uses valving between the individual biochips and components so that the valves can be opened or closed manually or automatically through an operating system. In an embodiment of the present disclosure, the human-on-chip plate is a hybrid-controlled organ-on-chip.

The human-on-chip plate could be created by associating different individual biochips together by interconnecting different biochips through its ducts to inlet/outlet valves on a biochips and valve holder plate, similar to a puzzle or modular arrangement. The human-on-chip plate could also incorporate different biochips in the same chassis, instead of separate components, with valves that could be assembled within the biochip while assembling the other components. The valves connecting the different biochip tissue growth channels could be actuated mechanically, hydraulically, pneumatically, magnetically, electrically, or any other actuation type. Any valve type could also be incorporated into this system.

The human-on-chip plate could also include open/close valves, which may typically be in a closed position, and waste removal channels incorporated within the biochip for its potential usage when assembled within the operating system. The cover glasses covering the individual biochip units could be made of one or more glass plates covering the surface channels of each entity, or only one glass case could be used to cover the full surface of the biochip instead. The interconnection between the different biochip entities could be through the duct openings straight along their axis, or it could go through the inlet/outlet holes to the surrounding biochips. The interface of the biochip with the operating system could be through the same inlet/outlet holes used in manual use, or through different holes that are separate from the system by normally closed valves that are actuated by the operating system.

In an embodiment, the presently disclosed system includes means for rotating the human-on-chip plate about a vertical and/or horizontal axis to ensure homogenous distribution of reagents within a solution, and the homogeneous distribution and attachment of cells post seeding in the channels.

In an embodiment, the human-on-chip plate allows for individual biochips to be connected to each other to form a series of interconnected biochips. Additionally, in an embodiment, specific biochips can be selectively disconnected from the remaining interconnected biochips to create multiple biochip circuits. As discussed later in the present disclosure, modulation of a fluid control mechanism allows for specific valves to be either opened or closed to facilitate numerous combinations of interconnected biochip circuits. The present disclosure allows for a plurality of protocols to be performed in parallel within a single human-on-chip plate because the modular nature of the human-on-chip plate allows for dynamically routing solutions to specific biochips. Dynamic routing of solutions in the presently disclosed system allows for specific biochips to be isolated from the remaining biochips to allow for precision medicine protocols to be performed.

In an embodiment, the human-on-chip plate fluidically connects multiple biochips to each other, and to other components of the operating system. These components may be, but are not limited to, any other biochip, a waste chamber, a sampling chamber, or a measuring device. In an embodiment, the connections can be altered at any time.

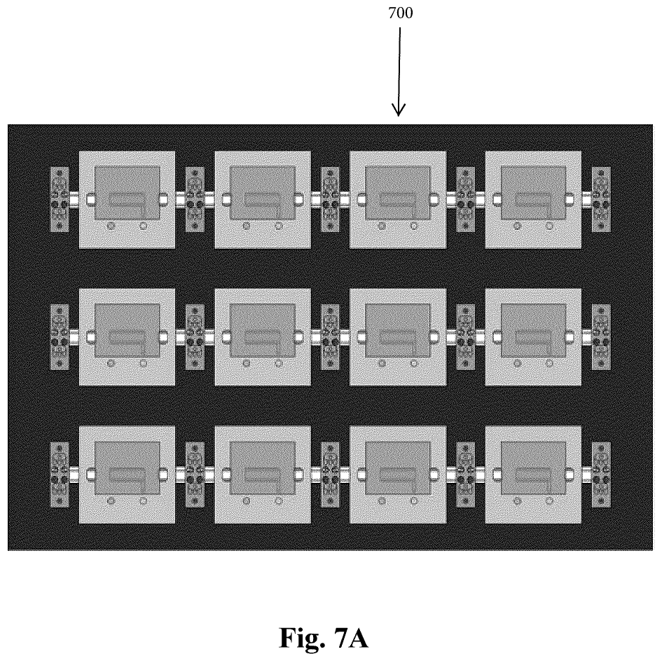

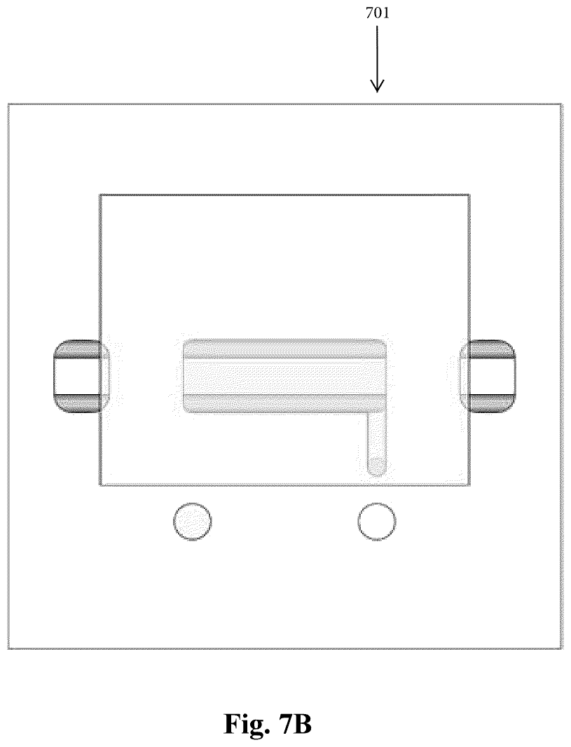

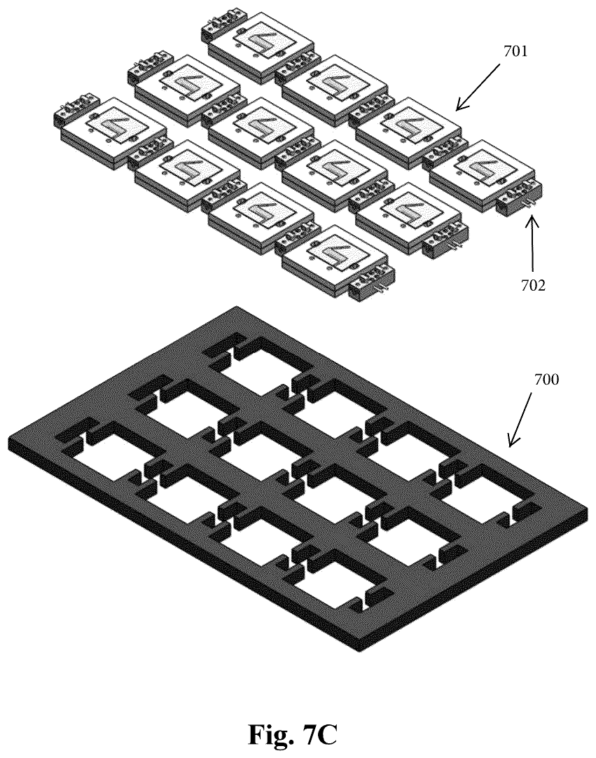

FIG. 7A-C shows the top and exploded views of the human-on-chip plate 700 and the individual biochip 701 respectively, according to an example of the present disclosure. In an embodiment, the individually removable biochips 701 are puzzled together by interconnecting different biochips 701 through its ducts 101 and 102 to inlet/outlet valves 702 on a human-on-chip plate 700. Fluid is pumped through the duct from the top of the human-on-chip plate and from a duct to another through a valving mechanism.

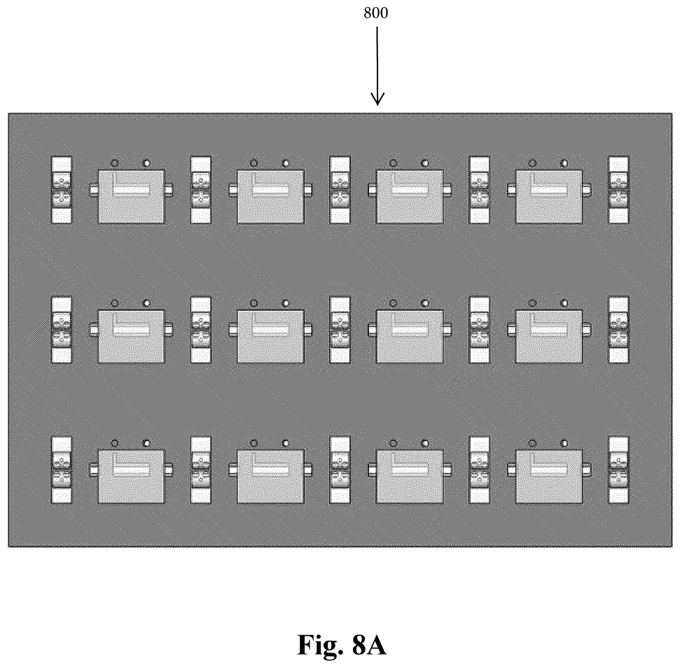

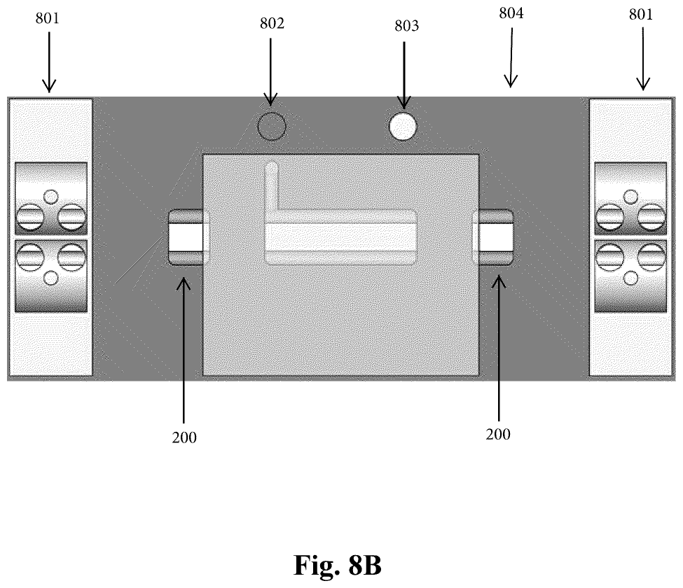

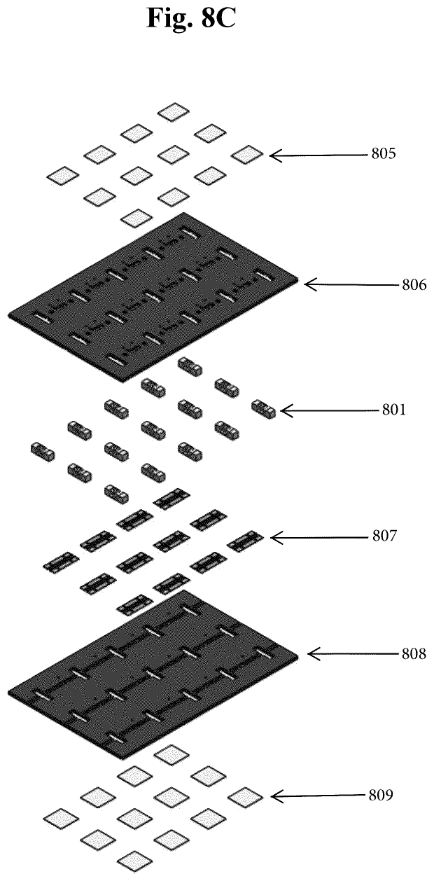

FIG. 8A-C shows the top and exploded views of the human-on-chip plate 800, according to an example of the present disclosure. In an embodiment, the individual biochip entities are incorporated as an integrated human-on-chip 800. Specifically, a cylindrical rotational mechanism valve 801 connects several integrated biochips 804, where the integrated biochip 804 is a membrane 807 sandwiched in between the top and bottom plates 806, 808, where the membrane 807 could include at least one porous cylindrical ductal scaffold channel surrounded by at least one stroma chamber, having a stroma feeding channel inlet and outlet 802, 803, and air filtering areas 200, and the stroma area is covered using an upper and lower glass cover 805, 809. In another embodiment of the human-on-chip plate 800, according to an example of the present disclosure, fluid is pumped through the side ducts of the human-on-chip plate 800 and from one duct to another through a valving system.

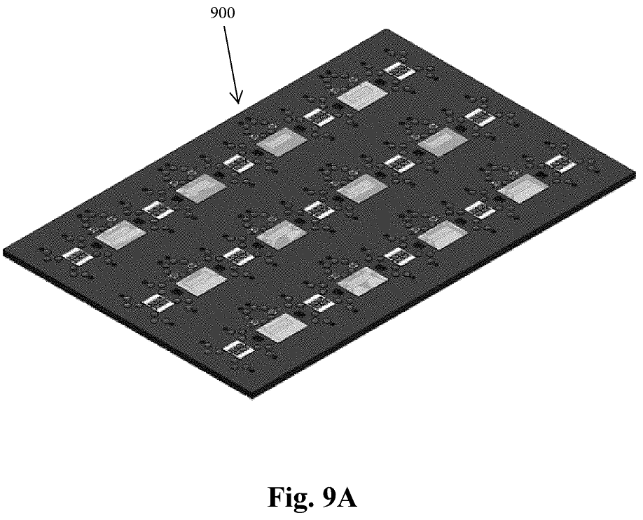

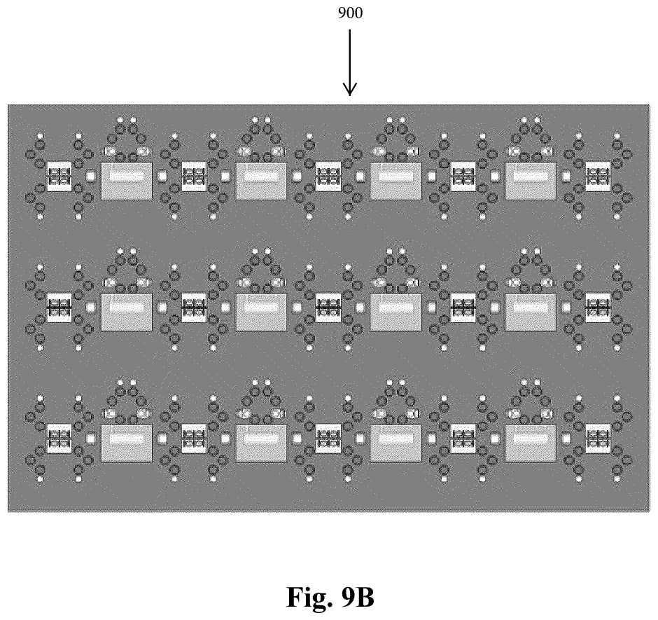

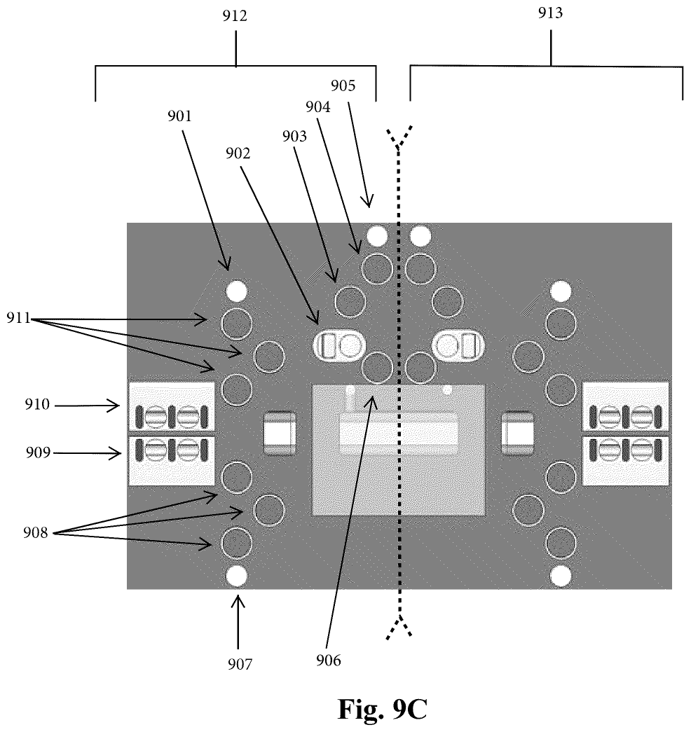

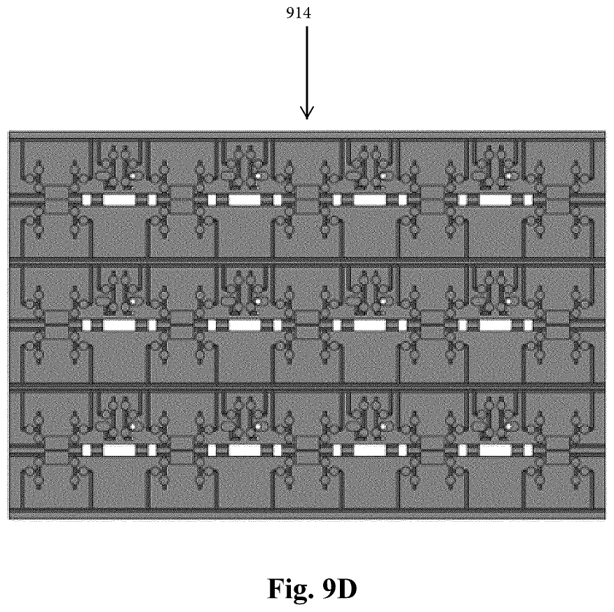

FIGS. 9A-D show top and perspective views of the human-on-chip plate 900, according to an example of the present disclosure. In an embodiment, the biochip entities are incorporated as one human-on-chip plate 900 that consists of several individual biochips where each individual biochip consists of two identical sections, inlet section 912 and outlet section 913, and each part comprises a first duct, one inlet/outlet hole and a separation valve 910, and a second duct, a second inlet/outlet hole and a second separation valve 909, where these entities connect the biochips to each other. In the biochip inlet section 912, a duct one valves set 911 and duct two valves set 908, where each set consists of three pneumatically actuated valves, where a main valve controls the sample taken from the duct inlet, and if opened, two valves control the flow of the sample towards the waste, or to the duct one inlet to operating system 901 and duct two inlet to operating system 907. In an embodiment, the biochip comprises a stroma inlet/outlet and a controlling valve 902, and a pneumatically actuated valve 906 controls the sample taken from the stroma inlet, and if opened, the flow of the sample is controlled by two pneumatically actuated valves, waste valve 903 that takes the sample to the waste, and a valve 904 to the stroma inlet to operating system 905.

In an embodiment, the individual biochip entities are incorporated as one human-on-chip plate 900 where a cylindrical rotational mechanism 801 connects the chassis and the valving mechanism to connect the individual biochip components, and the fluid is pumped through a duct from the top of the human-on-chip plate and from one duct to another through a valving system.

In an embodiment, a multiplex logic micro-valve system can actuate separate groups of valves, in a logic-coded format. For instance, the in-plate valves that connect the channels of adjacent chips within the HOC plate 900 can be micro-channeled and multiplexed together. Another system can run in parallel to multiplex the valves in the HOC that pump fluid from micro media chambers to the individual chips in the HOC 900.

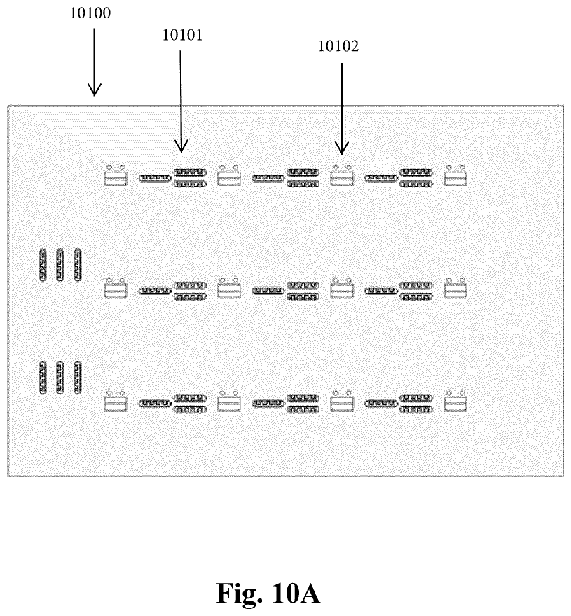

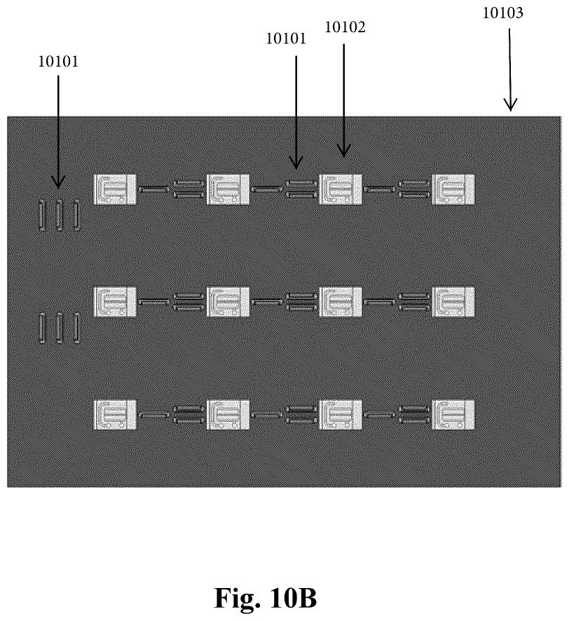

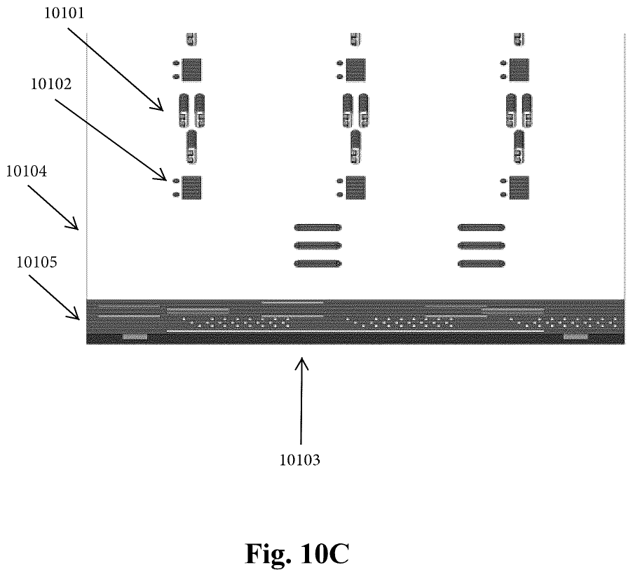

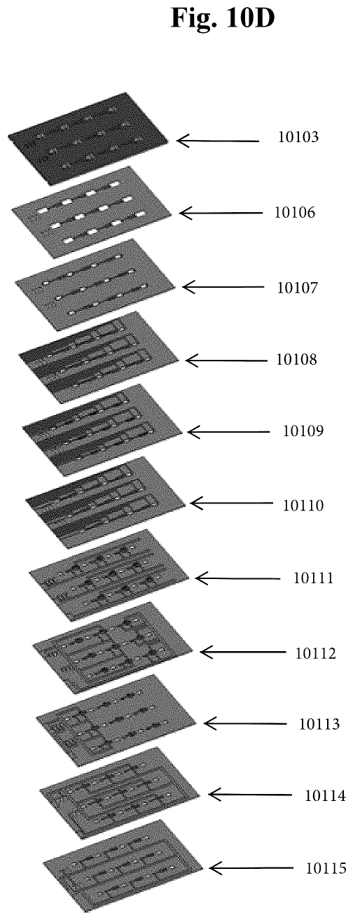

FIG. 10A-D shows the top, detailed, and exploded views of the human-on-chip plate 10104, according to an example of the present disclosure. In an embodiment, the individually removable biochips 10102 are puzzled together by interconnecting different biochips through ducts to inlet/outlet valves 10101 on a valve holder plate 10106, 10107. In an embodiment, the in-plate valves that connect the channels of adjacent chips within the HOC plate 10104 are actuated through a multiplex logic micro-valve system. Multiplexing of microvalves is based on multi-signal microvalves that require multiple separate signals to open, and a matrix of microchannels passing signals to the microvalves. The human-on-chip plate 10104 is made up of several plates stacked on top of each other, comprising a heating plate 10103, human-on-chip valve plate one 10106, human-on-chip valve plate two 10107, human-on-chip first duct channeling plate 10108, human-on-chip stroma channeling plate 10109, human-on-chip second duct channeling plate 10110, human-on-chip multiplex logic micro-valve system plates 10105 that comprise five plates, plate one 10111, plate two 10112, plate three 10113, plate four 10114, and plate five 10115. The heating plate 10103 contains a fluidic channel that passes in the inlet and the outlet of the top of the biochip 10102 and fits into the operating system, wherein any liquid of any temperature can be pumped to this heat plate, in order to control, increase, decrease or maintain, the temperature of the cell culture.

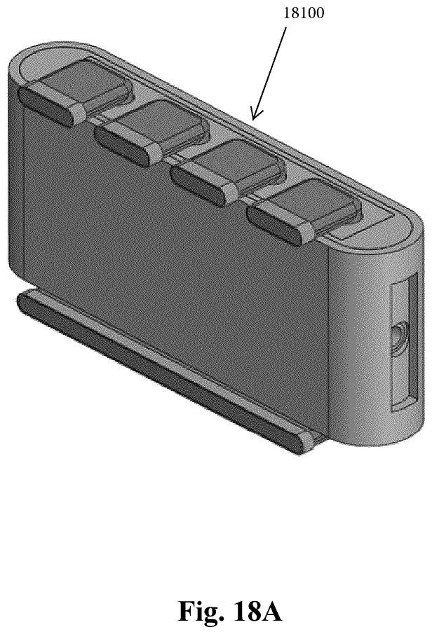

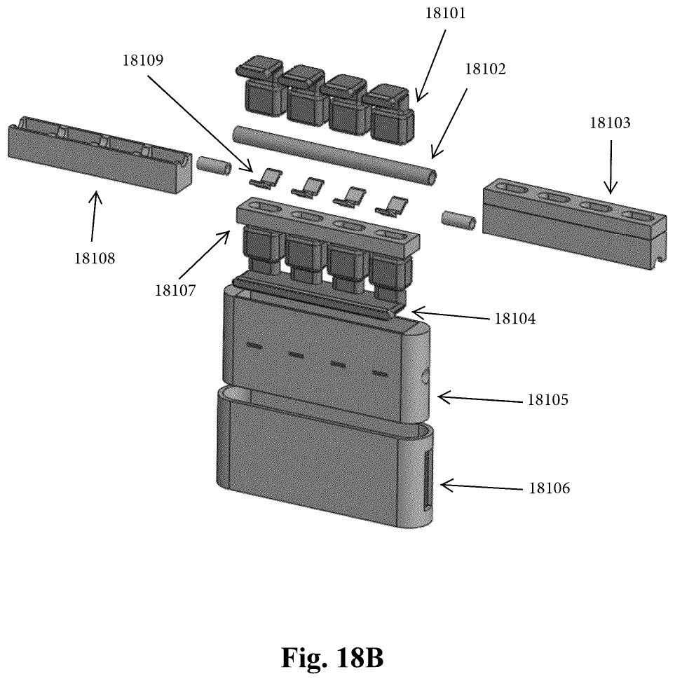

A human-on-chip multiplex logic micro-valve system plate 10111 is connected to a first bistable beam of each valve on the same row (three Rows, three signals). Another human-on-chip multiplex logic micro-valve system plate 10112 is connected to a second bistable beam of each valve on the first two columns (two columns, two signals). A third human-on-chip multiplex logic micro-valve system plate 10113 is connected to a third bistable beam of each valve on the second two columns (two columns, two signals). The fourth human-on-chip multiplex logic micro-valve system plate 10114 is connected to a fourth bistable beam of each valve in each biochip (two ducts and one stroma, three signals). In one embodiment of the multiplex logic micro-valve system, such as the embodiment of FIGS. 18A-B, all the valves can be closed by actuating one signal for all valves, through the bottom balloon channel 18104 that surrounds the bottom side of all the bistable beams 18109 of each valve. Each bistable beam 18109 is actuated to the open position, through a singular upper balloon 18101 that surrounds the upper side of each bistable beam 18109. Each individual beam is channeled and connected to another set of beams in a specific arrangement, creating the multiplex logic micro-valve system.

In one embodiment of the multiplex logic micro-valve system, bistable beam one is characterized to indicate the position of the valve on a specific row. For instance, in all the valves in row one, the first beam is connected through the plate channels to one signal indicating row one. Another signal, row two, goes to the first beam of the valves in row two. A third signal, row three, connects the first beam of valves in row three. Thus, there are three signals that could act on the valves of different rows (one, two, or three) that could open the beams of valves in either of those three rows. The second beam and third beam of the valves in this embodiment are connected to the channels along columns one or two and three or four respectively. So that there are two signals acting on beam two to open the valves on either columns one or two, and there are two signals acting on beam three to open the valves on either columns three or four. In an embodiment in combination with previously described embodiments, beam four is connected to valves channeled to either of the biochip 100 micro-channels types, duct one 101, duct two 102, or stroma feeding channel 401, so that there are three signals acting on beam four to open the valves on either duct one, duct two, or stroma feeding channels.

In one embodiment of the multiplex logic micro-valve system, the beams where no logical signal is acted on them such as beam two of columns three and four or beam three of columns one and two are all signaled to open together in one signal after initiating the valves by normally closing them using the common bottom balloon channel signal.

In one embodiment of the multiplex logic micro-valve system, all of the above signals work together in a logic sequence, such that to selectively open only the valve of row one, column one, duct one, a signal coded (1, 1, P, 1), for example, is initiated so that all the beams on this valve are open and the flow can be accessed through this valve. This code indicates the beams position on each valve (beam one, beam two, beam three, beam four), and the signal indicates the signal acted on the coded valves for each beam. Those four signals can trigger and open a selected valve through the multiplex channeling through a code of (Row number, Column one or two number, Column three or four number, Chip channel number), for example. Where row number could be one, two, or three acting on beam one. Beam two column number could be one, two, or “P” acting on beam two. Beam three column number could be three, four, or “P” acting on beam three. Chip channel number could be one, two, or three acting on beam four. The row and column number indicates the position of the valve on the plate in a 2D view, the biochip channel number indicates which valve at this location is to be triggered (duct 1, duct 2, or stroma), and “P” indicates the valves that are initially opened at the initiation of the valves after normally closing them in the bottom balloon valves signal.

In another embodiment of the multiplex logic micro-valve system, the logic system is made to reduce the number of signals to actuate as many valves as possible allowing for fitting and controlling a large number of valves with a minimum number of actuators. The mathematical logic beyond the multiplex system that allows for a specific number of switchable outputs “O” depends on the number of beams found on each valve “B”, the number of criterial connections “C”, and the normally reset state signal “N”. Such that the total signals acted on the system “S” is equal to the addition of “B”, “C”, and “N”, in which the total switchable outputs that the multiplex system allows “O” is equal to “B” multiplied by “C”. For example, for four beams located on one valve, and three connections criteria for each beam, and one normally reset signal, there would be 8 signals in total to control, resulting in a total of 12 switchable outputs.

In another embodiment according to an example of the present disclosure. The heating plate 10103 contains an integrated heating element for thermal control, in order to control, increase, decrease, or maintain, the temperature of the cell culture.

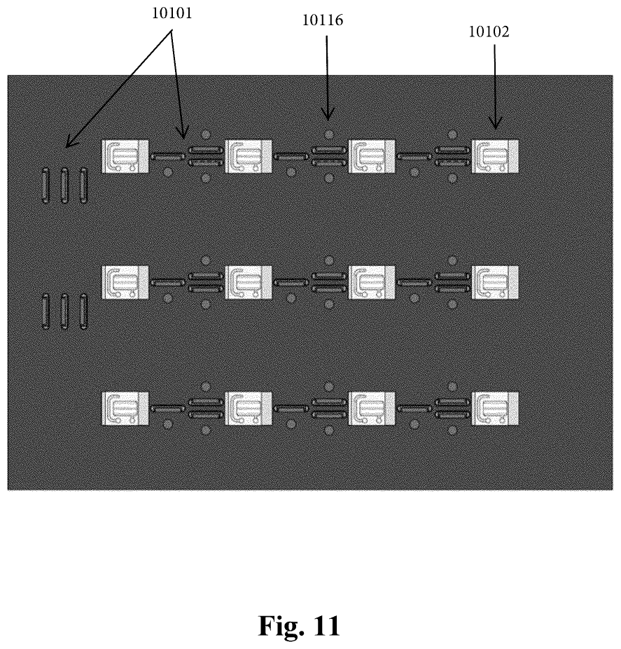

FIG. 11 shows the top view of the human-on-chip plate 10104 of FIGS. 10A-D, according to an example of the present disclosure. In an embodiment, the individually removable biochips 10102 are puzzled together through interconnecting different biochips through its ducts to inlet/outlet valves 10101 on a biochips and valve holder plate, and the fluid is sampled from a specific sampling spot 10116 inside each biochip in the human-on-chip plate, where a pipet-like mechanism can be used, or implemented in the system in order to take samples.

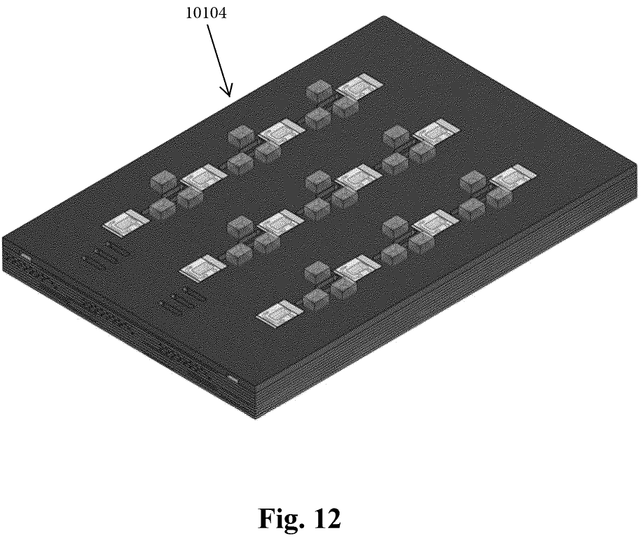

FIG. 12 shows a perspective view of the human-on-chip plate 10104 similar to that of the embodiment of FIG. 11, according to an example of the present disclosure. In an embodiment, the individually removable biochips similar to those of 10102 in FIG. 11 are puzzled together through interconnecting different biochips through ducts to inlet/outlet valves similar to those of 10101 in FIG. 11 on a biochips and valve holder plate, and the sample is taken on-the-spot through channels, chambers, and valves, where the cell samples can be either sampled on-the-spot using a pipet-like mechanism, pumped through a valving mechanism to a sampling chamber, or to the media chamber.

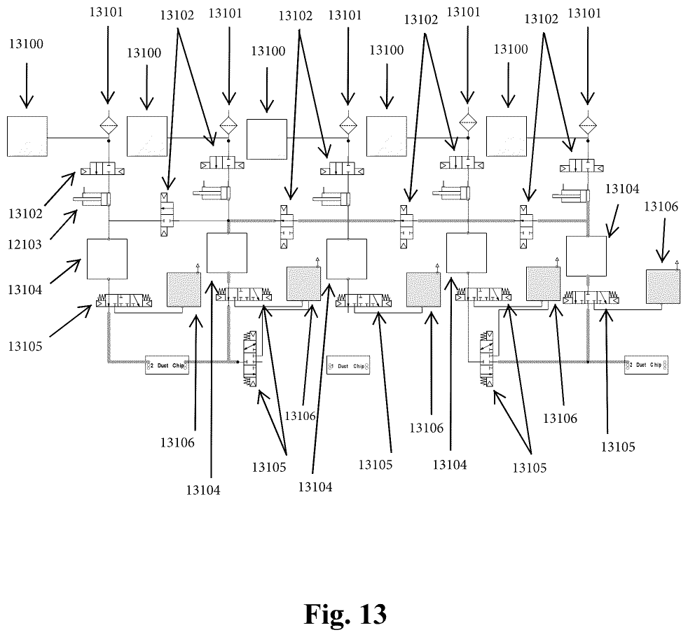

FIG. 13 shows a schematic diagram of an embodiment of the human-on-chip operating system. In this embodiment the media will be stored in the mixing chamber 13100, since this system is based on microchannels wherein the fluid will circulate, the formation of an air bubble will cause blockage in the system preventing any circulation from taking place, an air trap 13101 placed directly after the mixing chamber 13100 will remove the risk of bubbles formation blocking the flow. A set of 2/2 bistable pneumatically actuated valves 13102 will control the flow from the mixing chamber to the media chamber 13104. the fluid will pass through a pneumatically actuated piston 12103 that will extend and retract to pump the fluid, at this stage the 2/2 bistable pneumatically actuated valves 13102 will be actuating between an open/close position to prevent the backflow of the fluid. In this embodiment, a two-duct biochip can be connected to a one-duct biochip through the 3/3 spring-loaded pneumatically actuated valve 13105. The 3/3 spring-loaded pneumatically actuated valves 13105 are what connect all the different aspects of the human operating system, the media will be pumped by the pneumatically actuated piston 12103 from the media chamber 13104 through the 3/3 spring-loaded pneumatically actuated valves 13105 that actuated in the open position into the biochip 100 of the previously-described embodiments. The sample then can be extracted from the biochip 100 into the media chamber 13104, then the sample will travel through multiple 2/2 bistable pneumatically actuated valves 13102 and 3/3 spring-loaded pneumatically actuated valves 13105 into different biochips found on the human-on-chip plate 800 of the previously-described embodiments. In an embodiment, the fluid can be pumped through the side ducts of the human-on-chip plate and from one duct to another through a valving system.

In an embodiment, the fluid is sampled from a specific area inside each biochip in the human-on-chip plate.

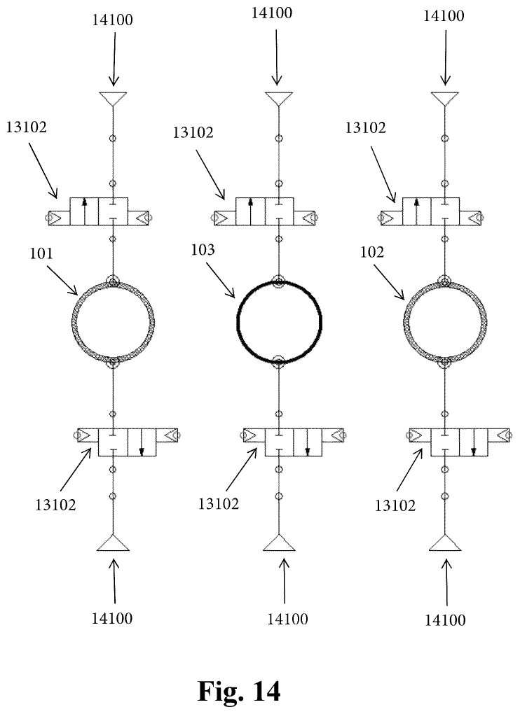

FIG. 14 illustrates a schematic diagram which contains pressure sensors at each biochip inlet and outlet fluid ports to manipulate the pressure inside the biochips' channels. In an additional embodiment, using a pressure sensor 14100 and 2/2 bistable pneumatically actuated valve 13102 that is situated at the inlet and outlet of the first duct 101, stoma 103, and second duct 102, the fluid is controllably perfused from the first duct 101 or the second duct 102 to the stoma 103 or vice versa.

In an embodiment, the valving means enable access to remove media samples during or after an experiment. The valving means enable introduction of media before or during an experiment. Additionally, the valving means enable control of where different media, especially cell types, are deposited during experimental set-up. In an embodiment, the valving means is stable in both the connected and disconnected state. Additionally, several other components can be utilized such as pinch valve with cam (friction held, or over-center profile), rotary valve (cylinder or ball valve with friction), snap-through bistable lever on pinch valve, and/or self-stabilized piloting pneumatic valve.

In an additional embodiment, the biological system on a biochip plate includes a number of valving means actuated by a number of actuator(s), wherein the number of actuator(s) required to actuate the valving means is fewer than the number of valving means. In an embodiment, the biochip is a multi-duct biochip. In an embodiment, the biochips are organ-on-chip biological simulations. In an additional embodiment, a first biochip simulating a first organ type is connected to a second biochip simulating a second organ type. In an embodiment, the biological system on chip plate simulates multiple organs of an organism. The biological system on biochip plate simulates sufficient organs to gain insight into the impact of experimental compounds on multiple organs within the organism. In an embodiment, the organism is human. In an additional embodiment, the biological system on biochip plate is configured to engage with external actuation and pumping means to enable a lower cost or even disposable interchangeable biological system on biochip plate.

### The Micro-Valving Mechanism

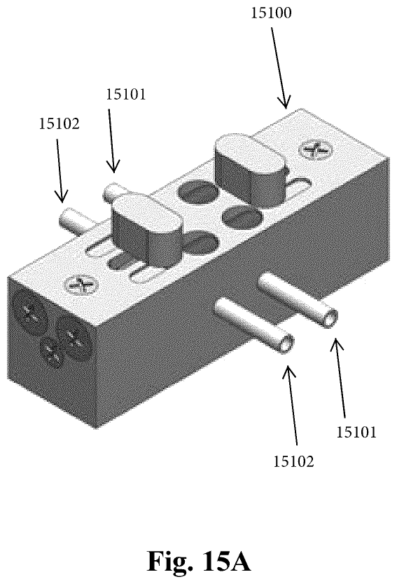

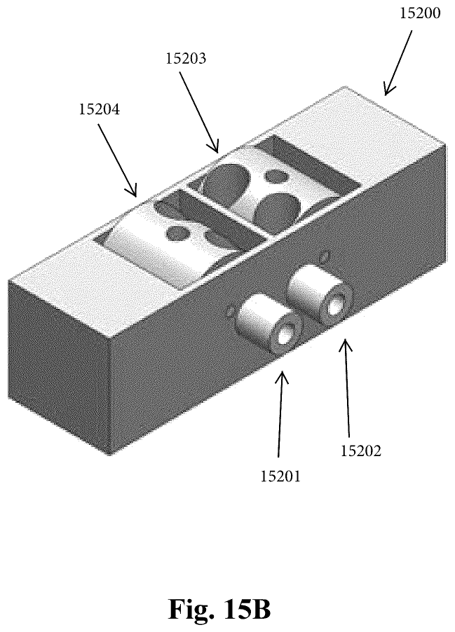

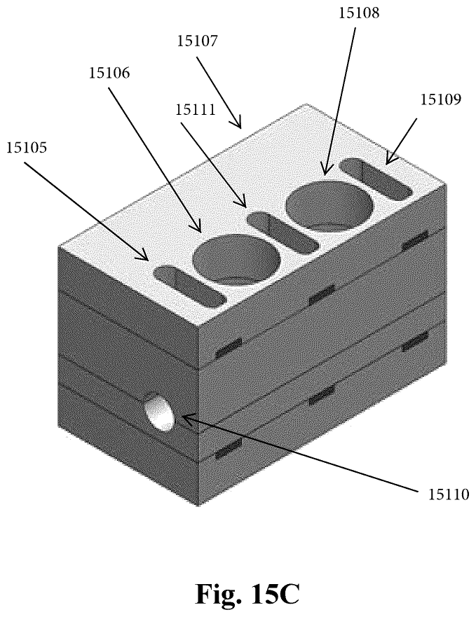

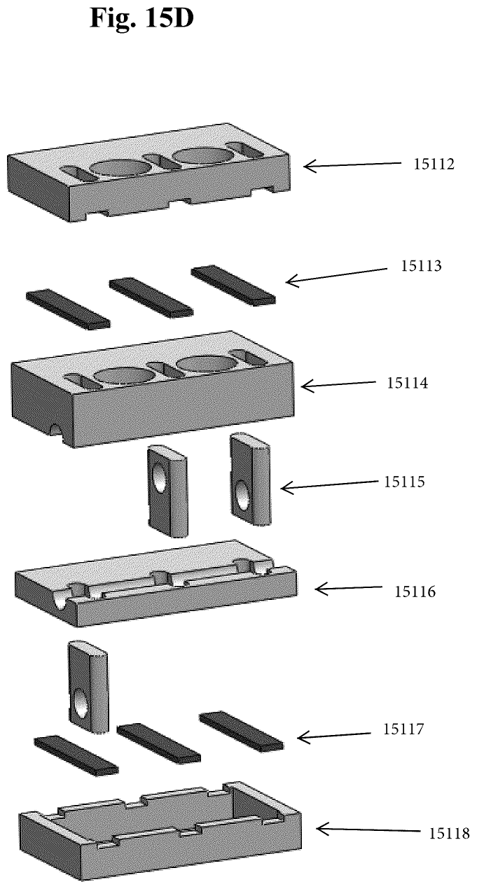

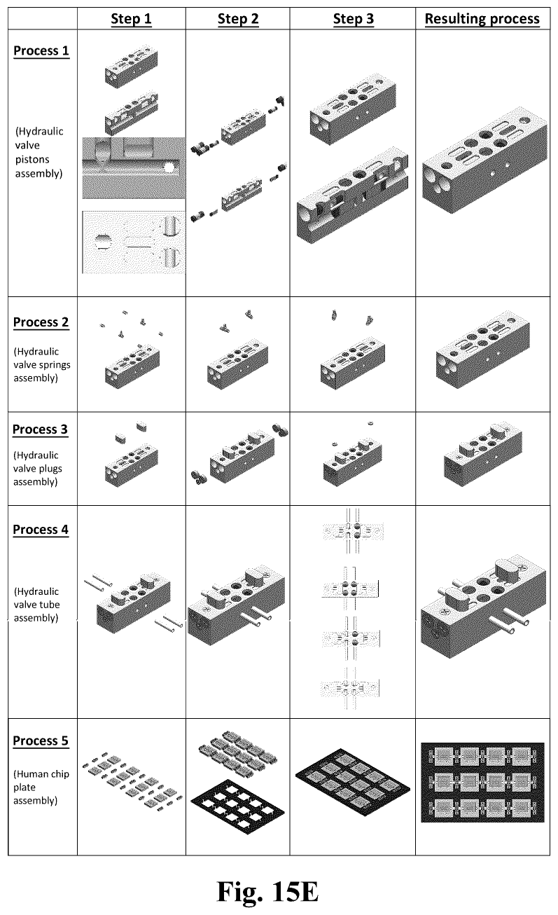

FIGS. 15A-E show a mechanical micro-valve 15100. In one embodiment, the mechanical micro-valve 15100 connects two adjacent biochips 100 together by connecting the ducts outlets on a first biochip to a next biochip inlets through the inlet/outlet connectors 15101, 15102. This embodiment of the mechanical micro-valve 15100 allows the exchange of any fluid passing through, or it can let the fluid pass through without interventions. This mechanical micro-valve 15100 has at least two states, the states switch between the valve allowing or blocking the fluid flow passing through lower channels. Two interlinked pistons situated inside the valve compartments move in opposite directions simultaneously controlling the state of the valve. In an embodiment the pistons can be hydraulically actuated. In FIG. 15E a step-by-step guide for this mechanical micro-valve 15100 is shown.

FIG. 15B shows a mechanical micro-valve 15100. The mechanical micro-valve 15100 connects two biochips 100 through the microfluidic channels. In one embodiment the mechanical micro-valve contains two drilled rotating cylinders 15203, 15204. Each cylinder has three channels, two channels located on the radial direction, and one axial channel located parallel to the cylinder axis. The axial channel connects the ducts of two adjacent biochips through the connection ports 15201, 15202. The radial channels reach halfway through the cylinder. The radial channels of each cylinder connect the fluid channels from the operating system to the inlet or outlet of the two adjacent biochips, while blocking the flow between the two adjacent biochips. The angle of rotation of the cylinders allows either the flow to pass from one biochip duct to the duct in the adjacent biochips or to block the flow between the ducts of the adjacent biochips and give access to the operating system channels to the biochip ducts separately. A small hole is located on the radial side of the cylinder in between the two radial channels and is used to control the angle of the rotating cylinders. The rotating cylinders 15203, 15204 maneuver in two positions. Position one is when duct one outlet 15101 from a first biochip 100 is aligned with the channel in the rotating cylinder 15204, 15203 reaching to the radial channel aligned with operating system channels. The fluid passes from the operating system through the duct without reaching the adjacent biochip. Position two is when duct one outlet 15101 from the first biochip is aligned with that axial channel in the cylinder, where the fluid would pass through the rotating cylinder 15203, 15204 to the adjacent biochip.

FIG. 15C-15D shows a pneumatically actuated drilled piston micro-valve 15107. In an embodiment, the pneumatically actuated drilled piston micro-valve 15107 is equipped with two top inlet ports 15106, 15108 that allow fluid flow into the duct port 15110, this valve can be pneumatically actuated through the air inlet one 15105, air inlet two 15111, and air inlet three 15109, wherein each air inlet controls a piston that's actuated between an open/closed position. Once the valve is actuated the three pistons will move into their open position wherein the drilled holes will align allowing fluid flow. A set of membranes 15113, 15117 above and below the pistons 15115 are housed inside the valve housing 15114. The membranes 15113, 15117 allow the airflow to actuate the pistons 15115 between open/closed positions. At the bottom side of the pneumatically actuated drilled piston micro-valve 15107, the duct port 15110 connects two adjacent biochips 100 together.

FIG. 15E shows a step-by-step guide on how to assemble the mechanical micro-valve 15100 shown in FIGS. 15A-D. Process one describes the assembly method of the hydraulic valve pistons, where process two holds the valve springs assembly that align and connect the two upper adjacent pistons. Process three shows the valve plugs assembly, that induce a seal-type effect. Process four introduces the valve tube assembly. Process 5 shows how this mechanical micro-valve 15100 is adapted in the human-on-chip plate 10104 with the biochips 100 connected.

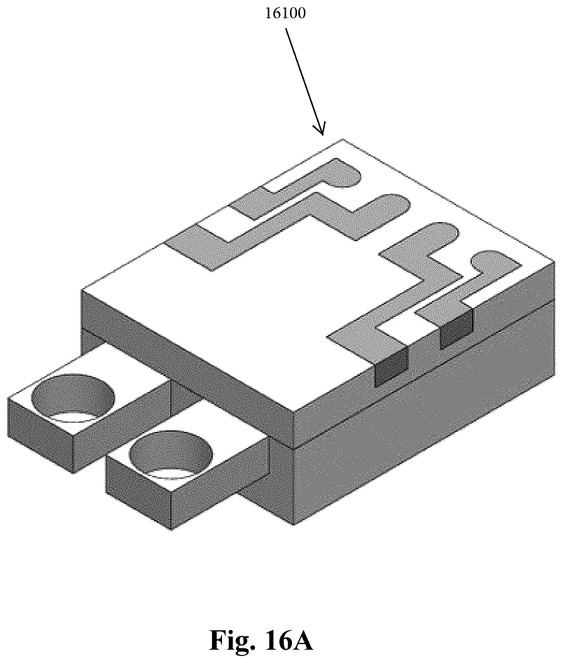

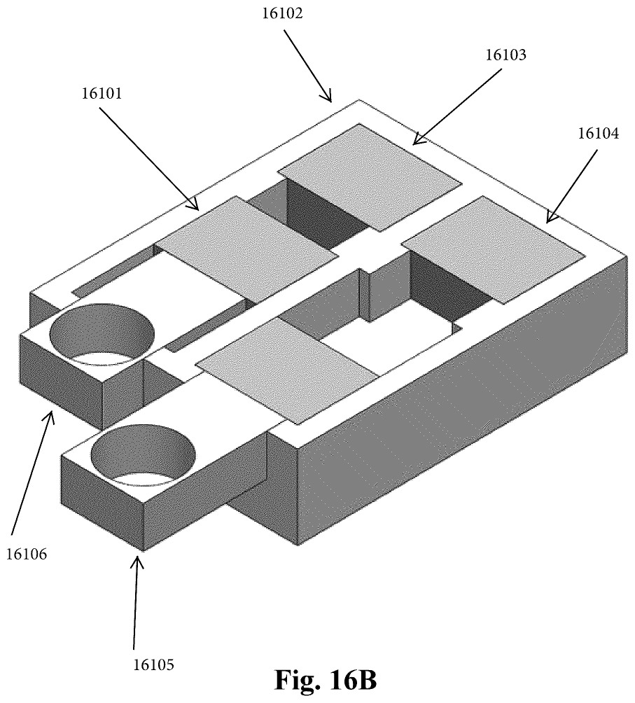

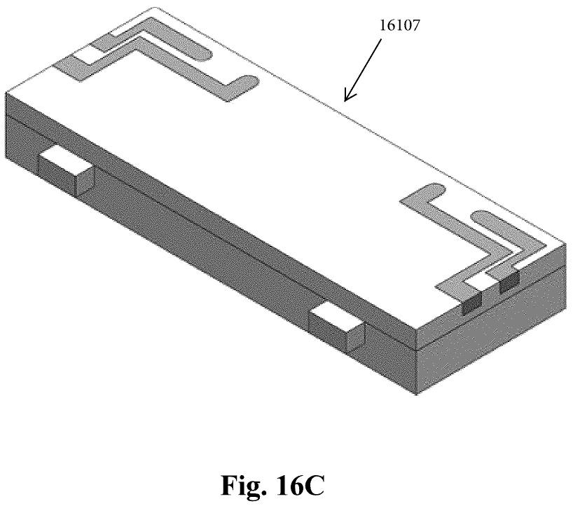

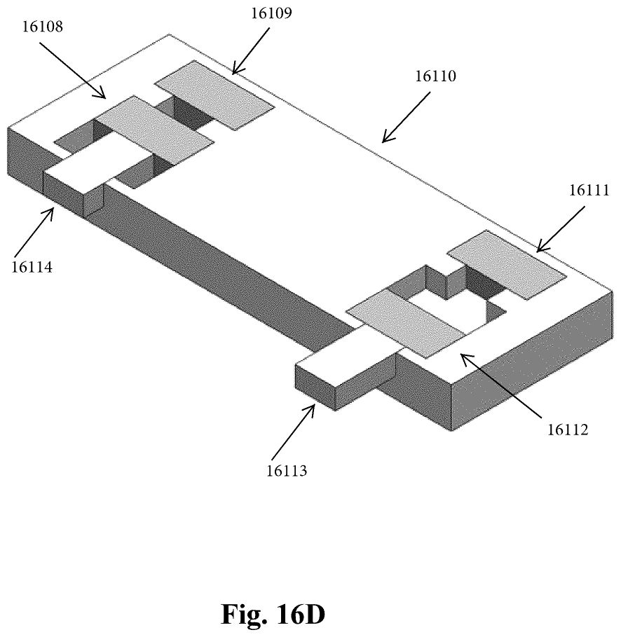

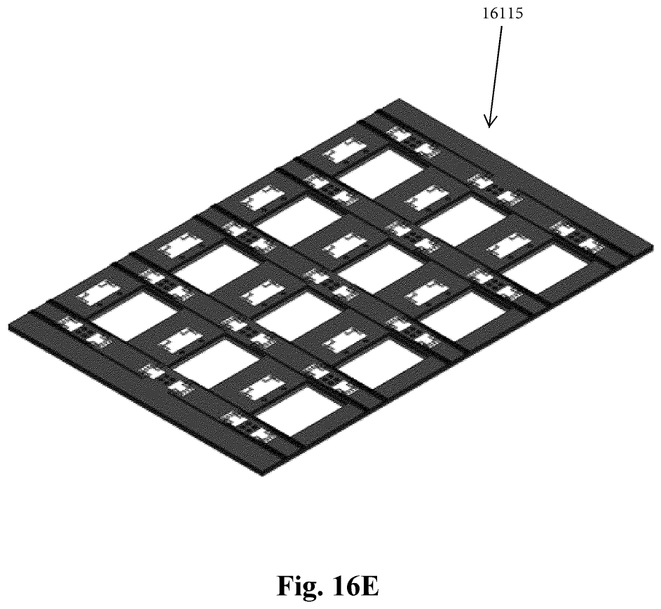

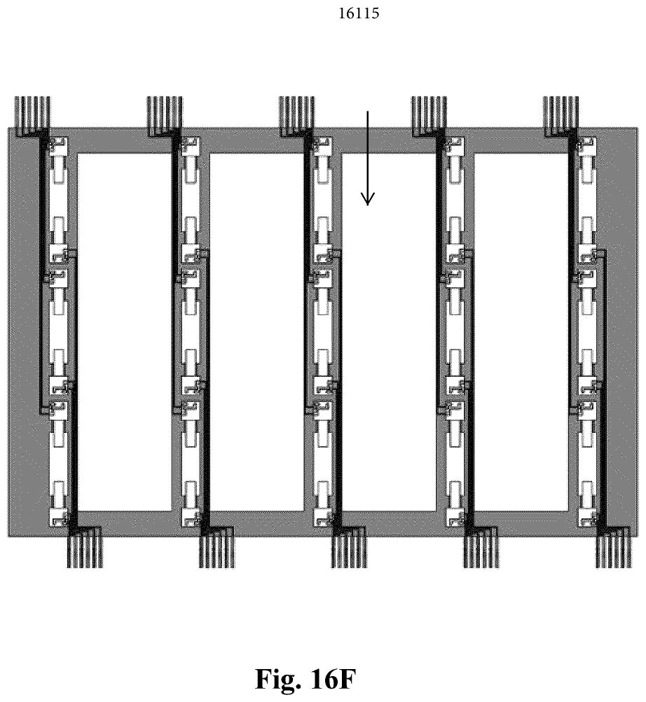

FIG. 16A-B shows an electrically actuated solenoid bistable micro-valve. This micro-valve contains two pistons 16106, 16105. The valve housing 16100 contains two fixed separate magnets 16103, 16104 that control the position of the actuation of the piston 16106, 16105. Electric signals trigger the magnet's force direction moving the pistons linearly between two locations. This movement opens the microfluidic channel or blocks it.

FIG. 16 C-D shows an embodiment of the electrically actuated solenoid bistable micro-valve 16107. The valve housing 16110 contains two fixed separate magnets 16109, 16111 that control the position of the pistons 16114, 16113. Electric signals trigger the magnet's force direction moving the pistons' magnets 16108, 16112 linearly between two locations. This movement opens the microfluidic channel or blocks it.

FIGS. 16E-F show the assembly of the electrically actuated solenoid bistable micro-valve 16107 on the human on-chip plate 10104. Wherein the plate 16115 in FIG. 16E shows the microfluidic channeling plate that the micro-valves intend to open or close. In addition, FIG. 16F shows the top view of the human-on-chip plate having the micro-valves and its electric connections 16115.

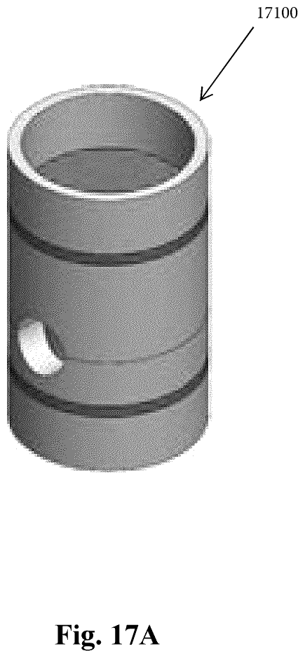

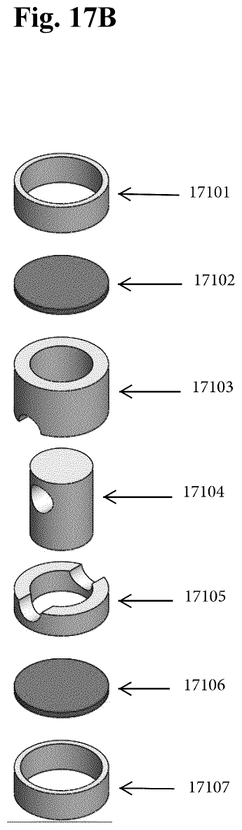

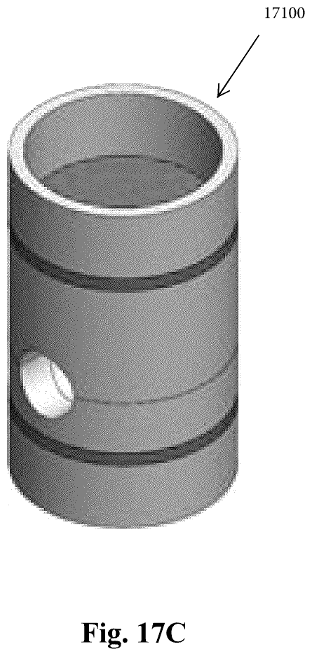

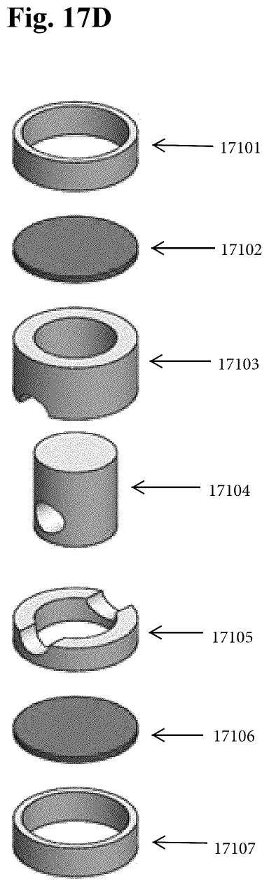

FIGS. 17A and 17C show different embodiments of the pneumatically actuated drilled piston micro-valve 17100. FIG. 17A shows a normally closed micro-valve 17100. FIG. 17C shows a normally opened micro-valve 17100. FIGS. 17A-D show the pneumatic actuated open-close membrane valve 17100, the ring 17101 and the top housing 17103 lock the top membrane 17102 in position, so when pneumatically actuated, the piston 17104 is pushed by the membrane to align the holes of the piston 17104 and the microfluidic channel. The lower membrane 17106 retracts the piston to its normal position. The bottom housing 17107 and the middle housing 17105 lock the lower membrane 17106 in position. When the hole of the drilled piston aligns with the hole in the valve, it opens to allow fluid flow. If the piston hole is not aligned it blocks the flow.

FIG. 18A-B shows a perspective view of the pneumatically actuated flexible tube micro-valve 18100 connecting adjacent biochip 100 channels in the human-on-chip plate 10104.

FIG. 18B shows an exploded view, according to an example of the present disclosure. In an embodiment, the valving mechanism connecting individual biochip 100 channels is bistable, where each valve contains four bistable beams, opening or closing the flexible tube 18102. Once actuated, the bistable beam 18109 will stay in one of those two bistable positions. The tube is placed in between the upper plate 18103 and bottom plate 18108, the bistable beam 18109 is located and interfaced with openings on plate 18107. The bottom balloon channel 18106 is pneumatically actuated, where when the air flows in the channel, it closes all the channels in a micro-valve. The upper set of singular balloons 18101, are individually pneumatically actuated, each one actuates the bistable beam below it, opening a portion of the tube that is below it. This mechanism is enclosed inside two covers 18105 and 18106 to support its interface with the human-on-chip plate 10104. The valve has four upper singular balloons 18101 and a normally set bottom balloon channel 18104 to allow for the multiplex valving system to be actuated with a minimum number of actuators.

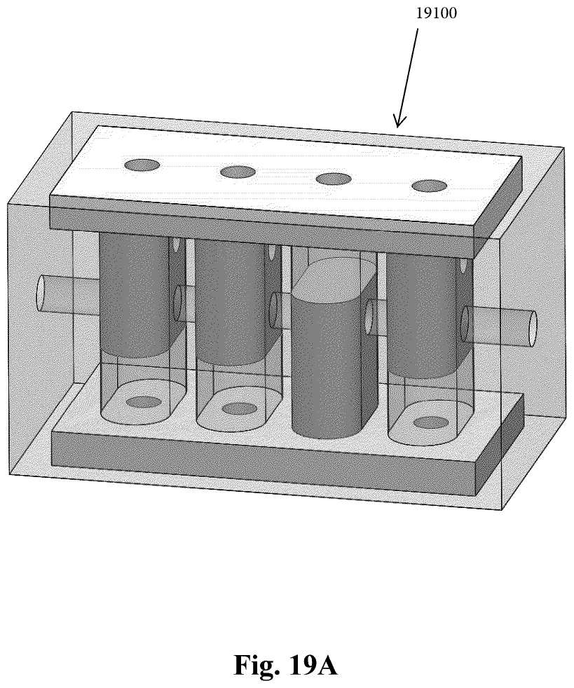

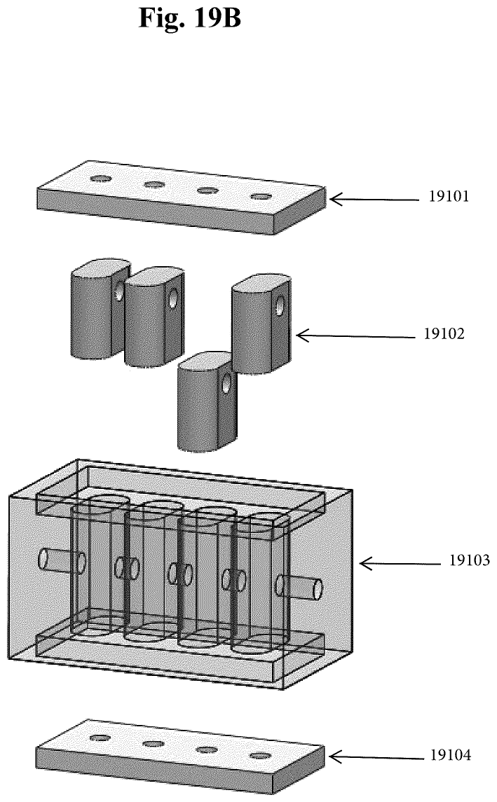

FIGS. 19A-B show a bistable pneumatically actuated magnetic drilled-piston micro-valve 19100, connecting adjacent biochips 100 of the human-on-chip plate 10104. FIG. 19B shows an exploded view of this valve. In an embodiment, the valving mechanism is bistable through the upper magnetic plate 19101 and the lower magnetic plate 19104. Once actuated, the holes of the drilled pistons 19102 will align with the channel inside the valve allowing fluid flow across the valve. The valve has four pistons to allow for the multiplex valving system to be actuated with a minimum number of actuators.

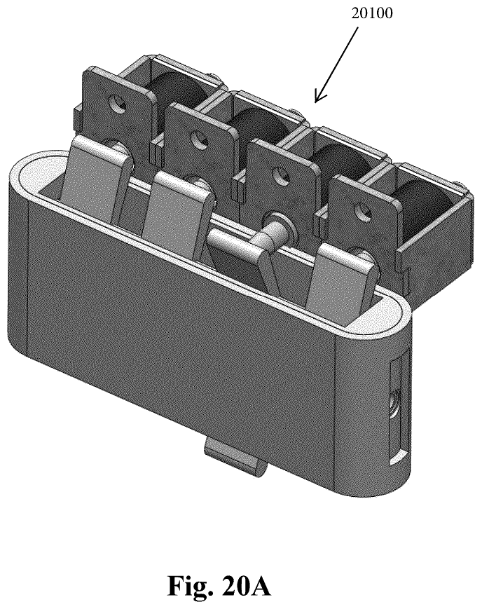

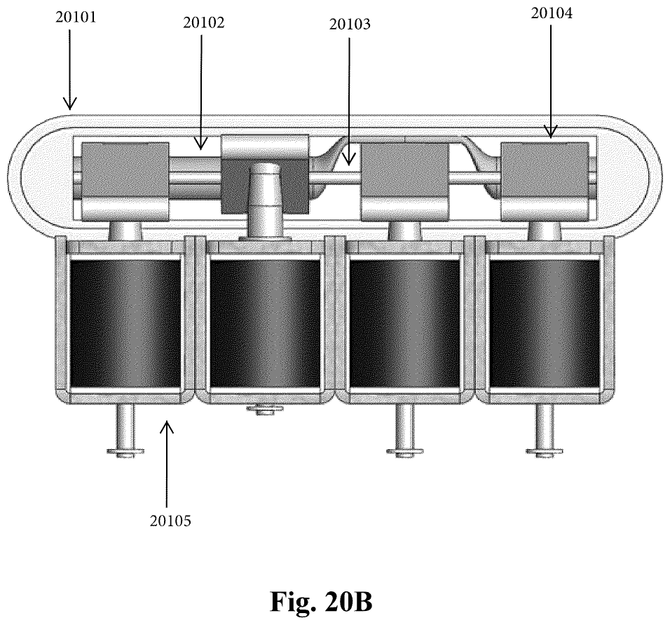

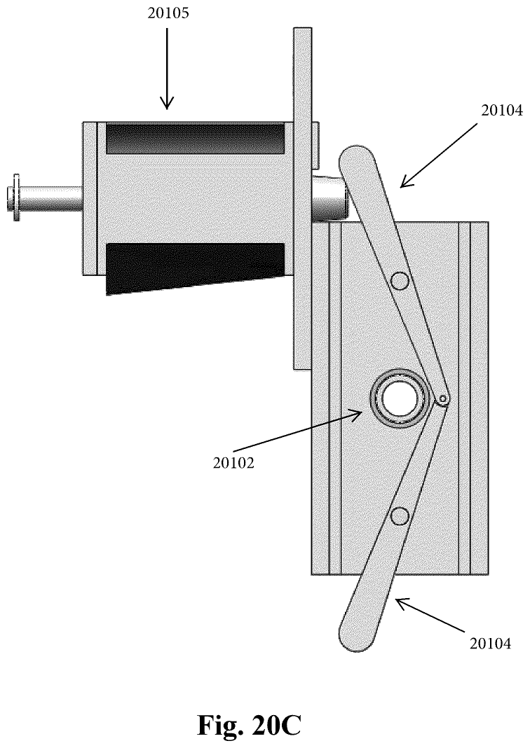

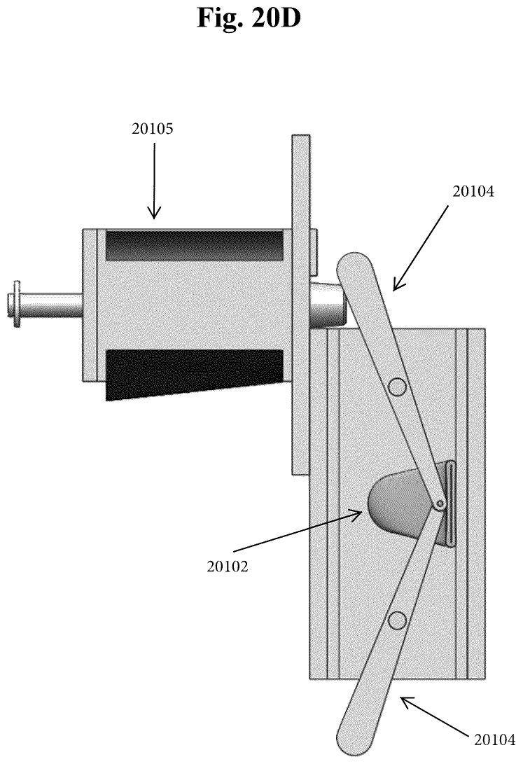

FIGS. 20A-D show a view of the bistable mechanically actuated flexible tube micro-valve 20100 connecting the adjacent biochip 100 channels of the human-on-chip plate 10104 through the microfluidic channels. The valving mechanism is bistable, where the micro-valve has two stable positions. At the initial position of the bistable beam 20104 the flexible tube 20102 is open and solenoid 20105 is not actuated. In the second position, the solenoid 20105 pushes a piston that actuates the bistable beam 20104 positions closing the flexible tube 20102 and blocking the flow in the channels 20105. The pins 20103 hold the bistable beams 20104 in position. The valve components are contained inside the valve housing 20101. This design can be solenoidally or pneumatically actuated in order to move the main beam.

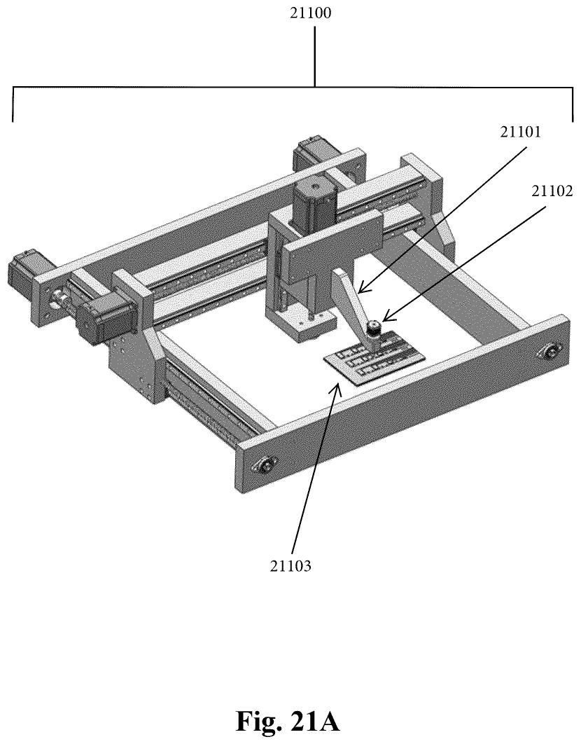

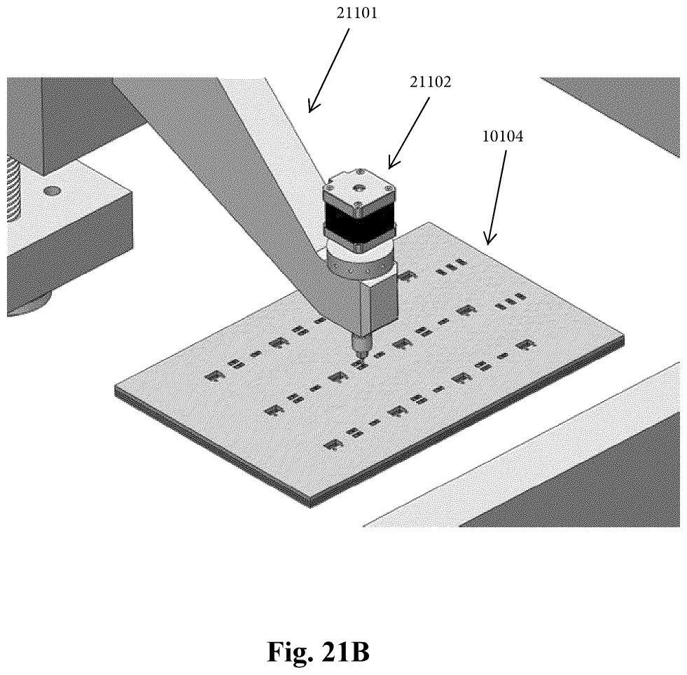

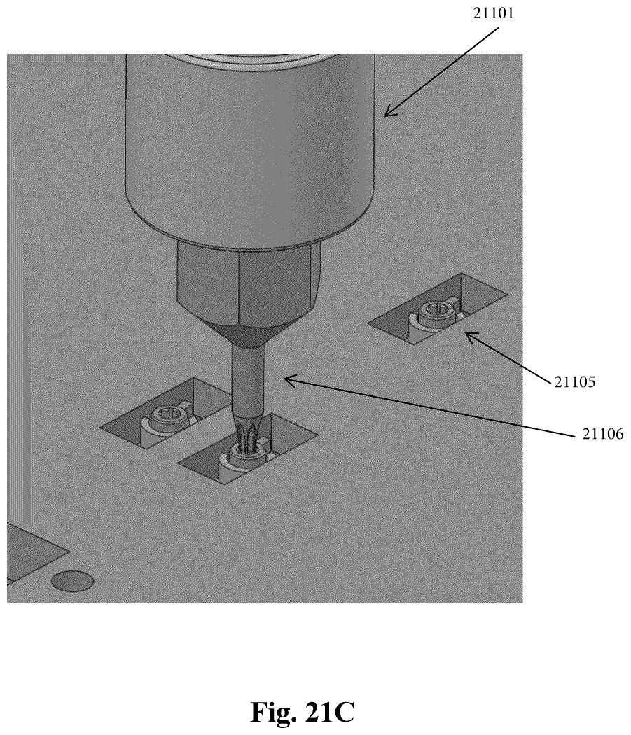

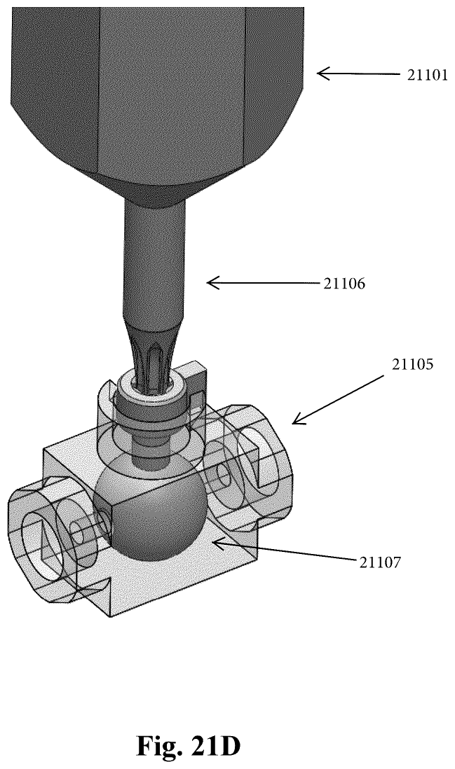

FIGS. 21A-D show the mechanically actuated pinched ball open-close micro-valve 21105 connecting the individual biochip 100 components of the human-on-chip plate 10104, according to an example of the present disclosure. In an embodiment, the valving mechanism connecting individual biochip 100 components is either open or closed, where a ball micro valve 21105 contained in the ball valve housing 21107, is a drilled sphere that can be rotated to either allow a fluid to pass or block it. The ball-micro-valve 21105 is mechanically actuated by a motor 21102 controlled by a system that can be actuated by a robot 21101 that moves in a 3D plane and opens or closes the ball-micro-valves 21105 using, for example, a torx bit head 21106, or in a standalone system that can preset the micro-valves 21105 configurations in order to mimic an organ in the human body.

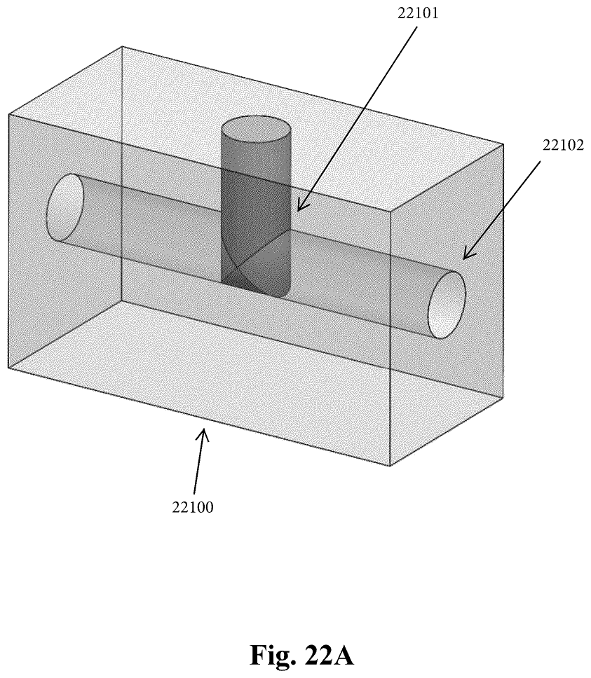

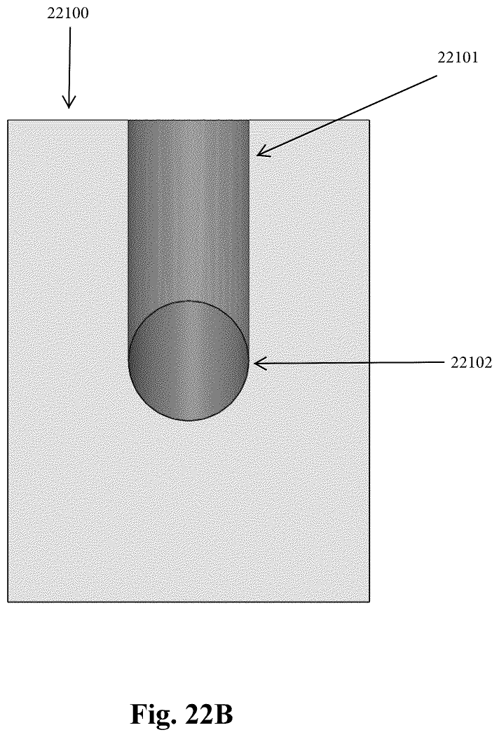

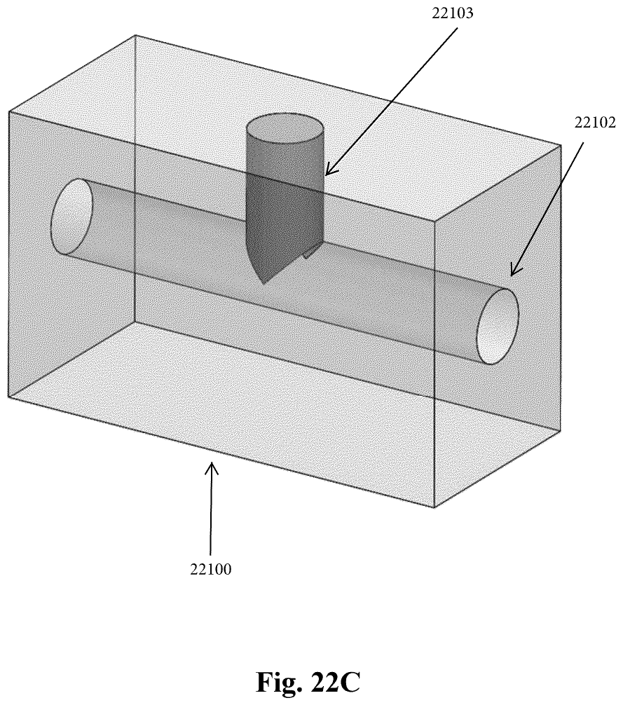

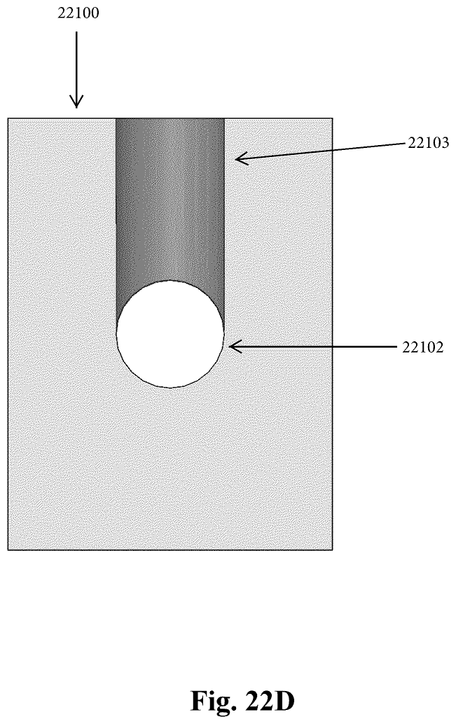

FIGS. 22A-D, show different permanent seal open-close micro-valve 22100 connecting the individual biochip components of the human-on-chip plate 10104, according to an example of the present disclosure. In an embodiment, the valving mechanism which connects individual biochip 100 components through its cylindrical holes 22102 is either open or closed, where the permanent seal is preset once to either have an opened or closed micro-valve, where a pin is permanently inserted into the channel, that can be either a blocking pin 22101 or an opening pin 22103, depending on the design of the pin.

### The Operating System Component Assembly

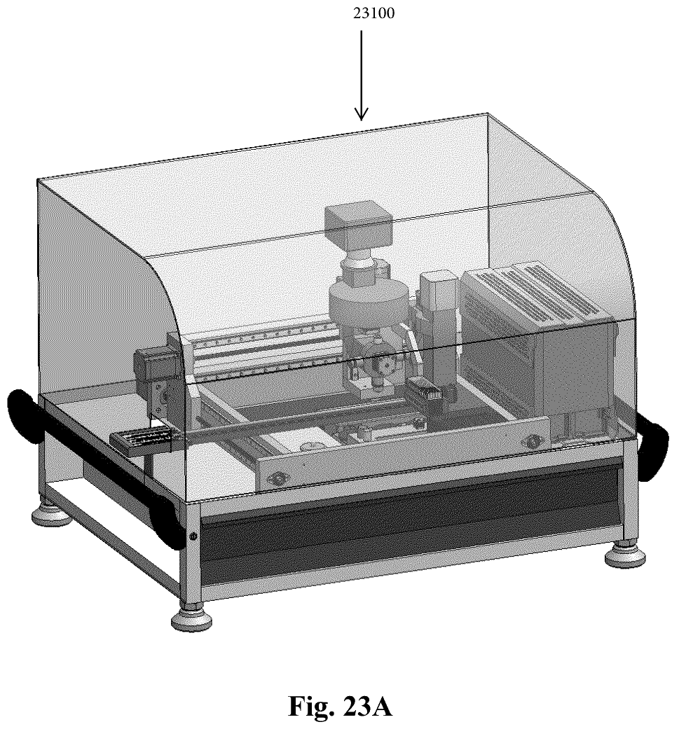

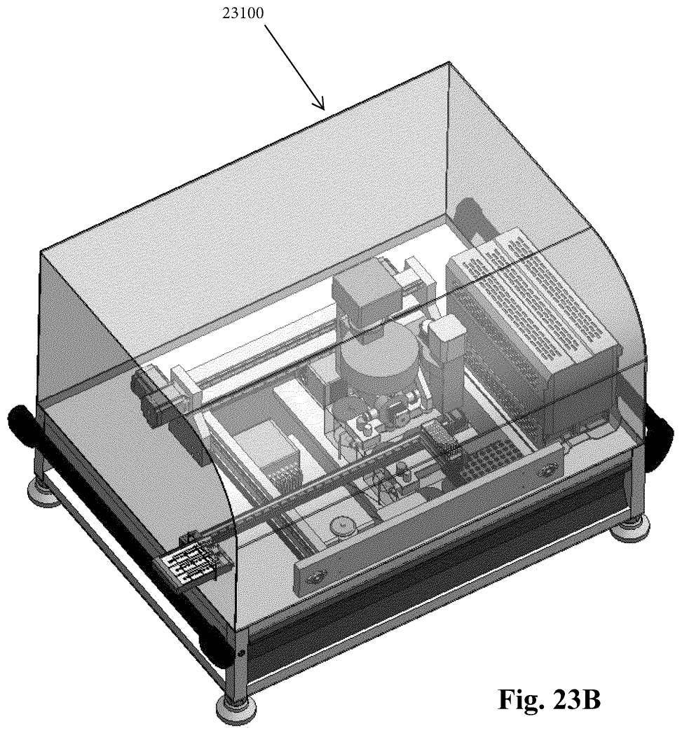

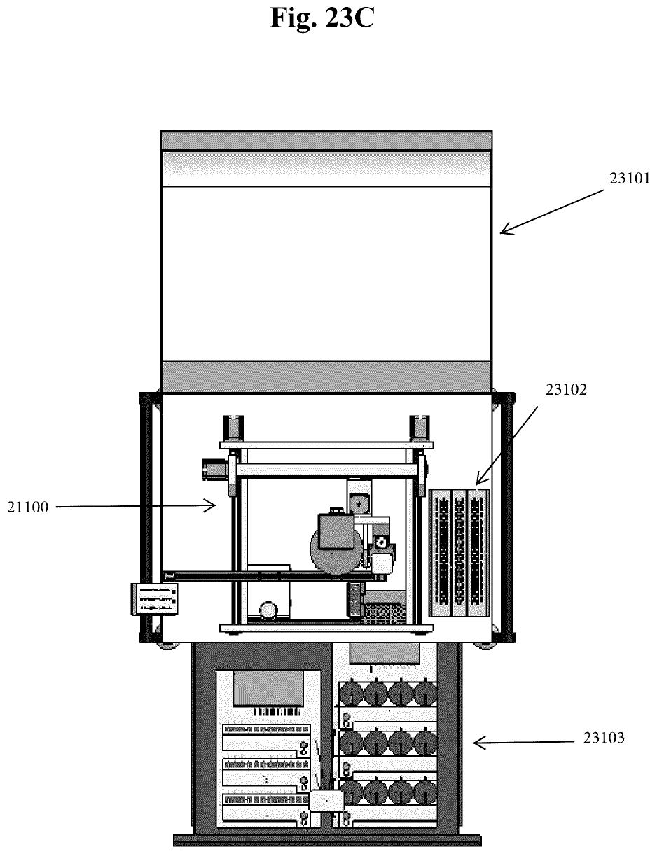

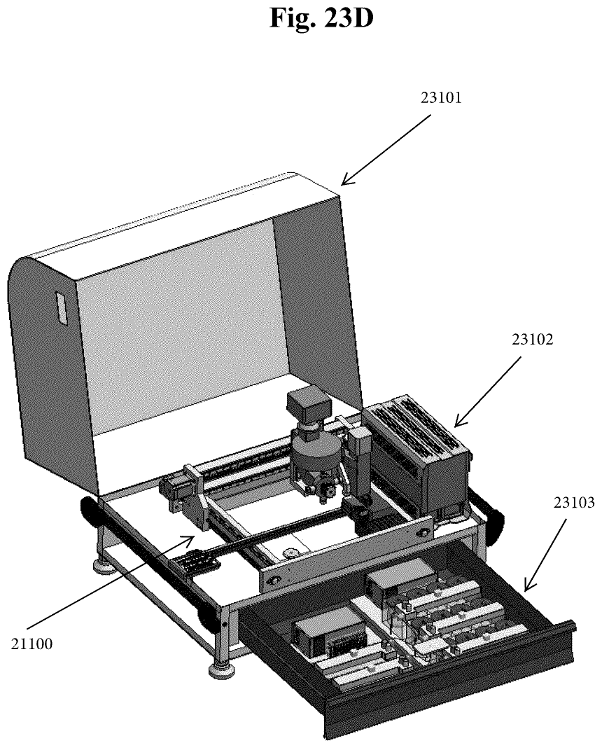

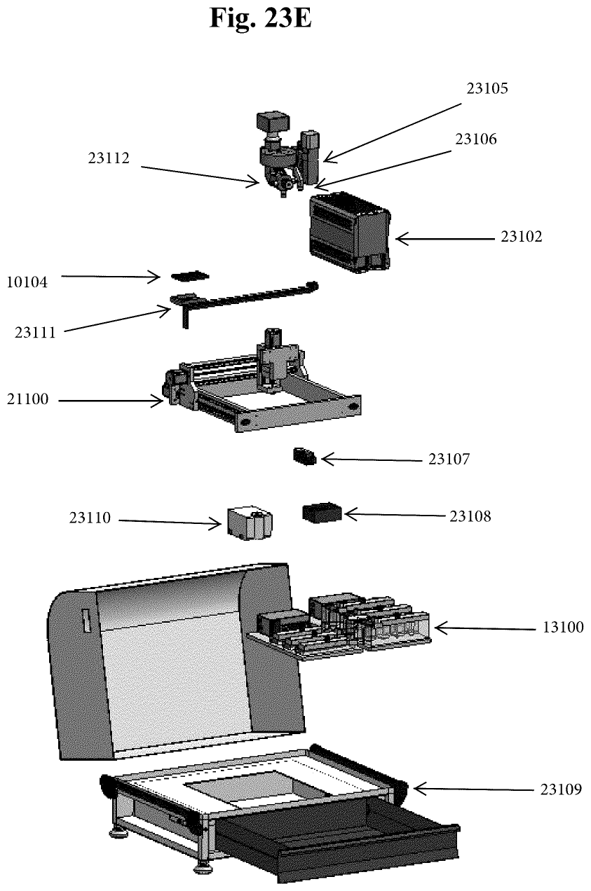

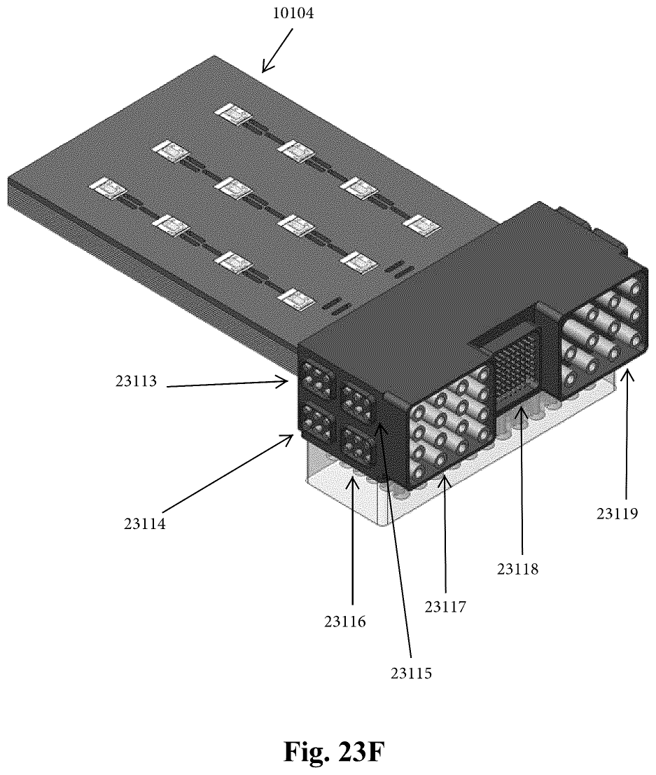

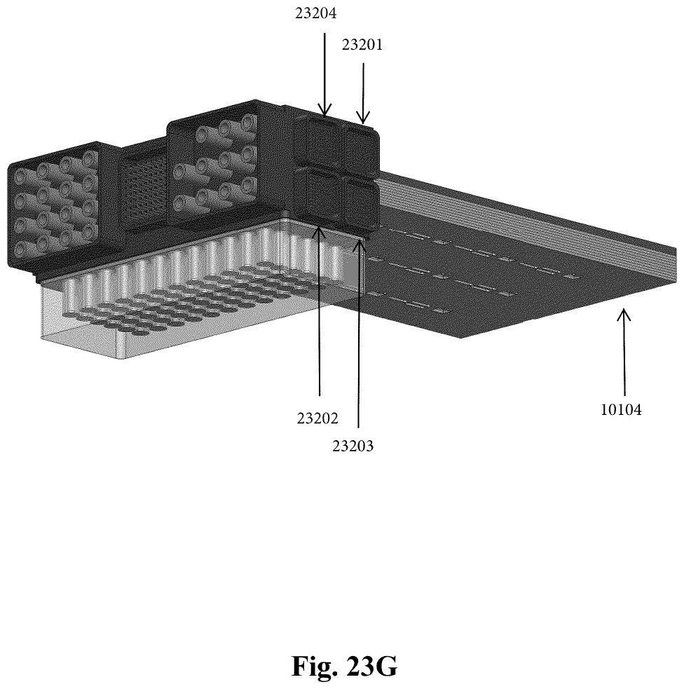

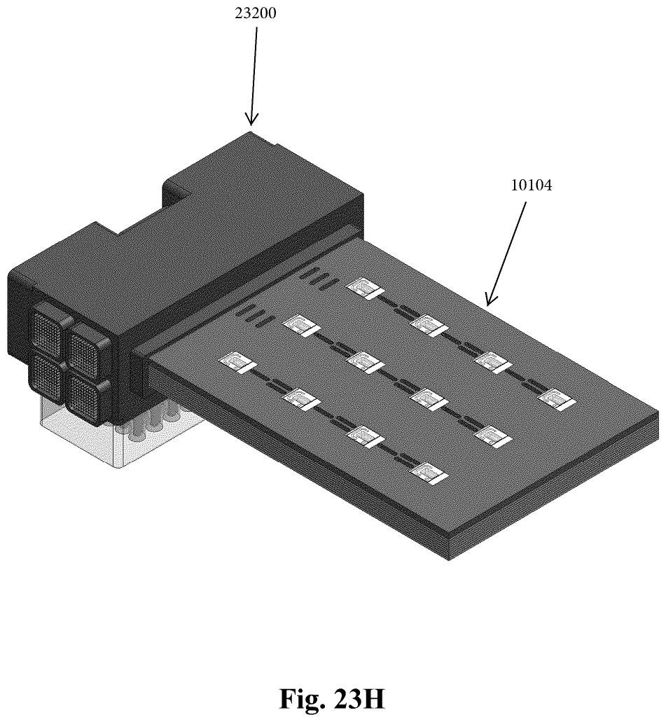

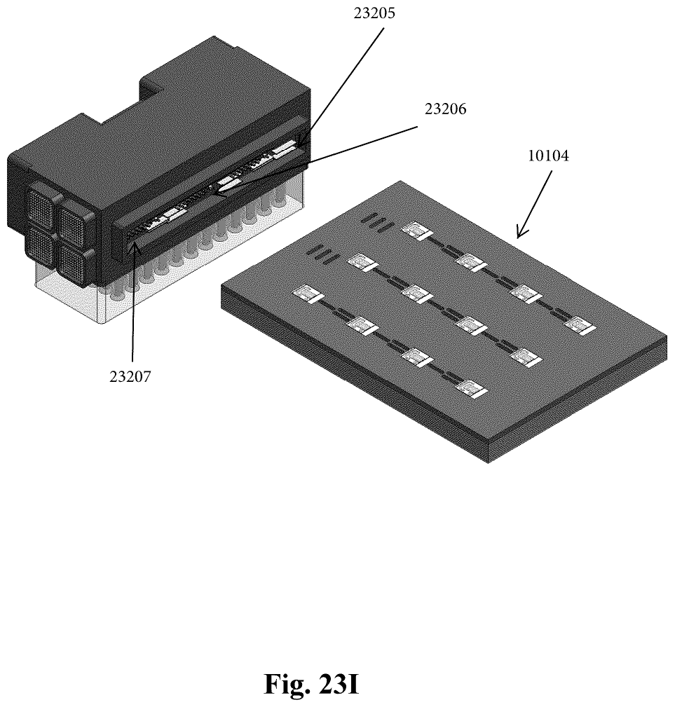

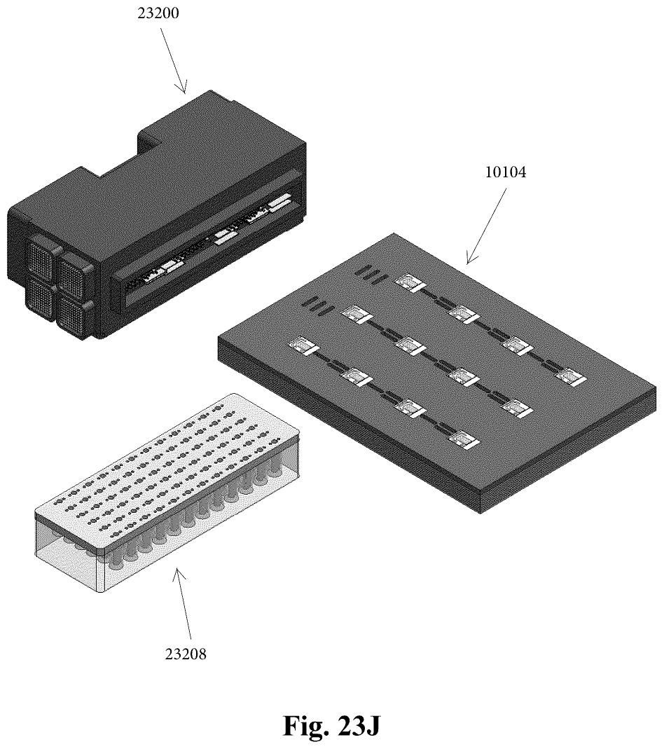

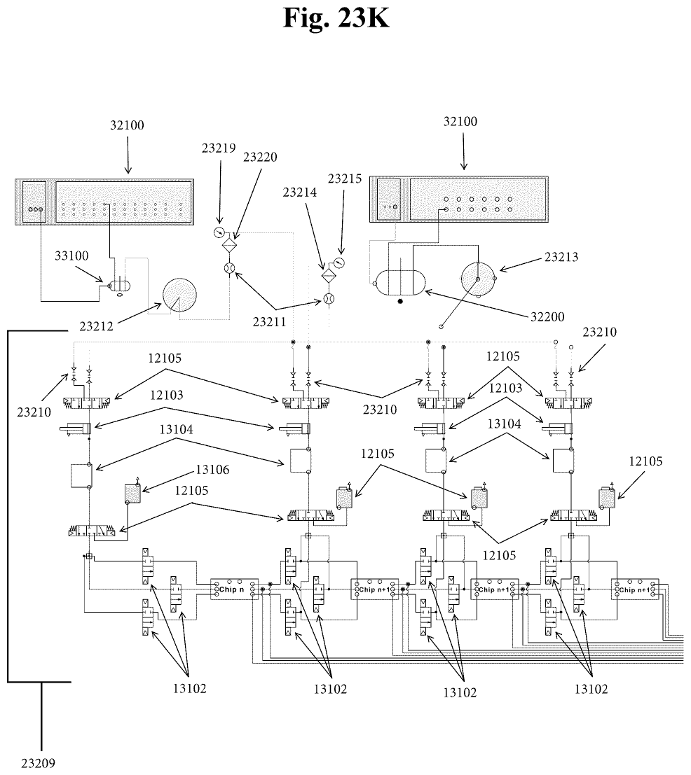

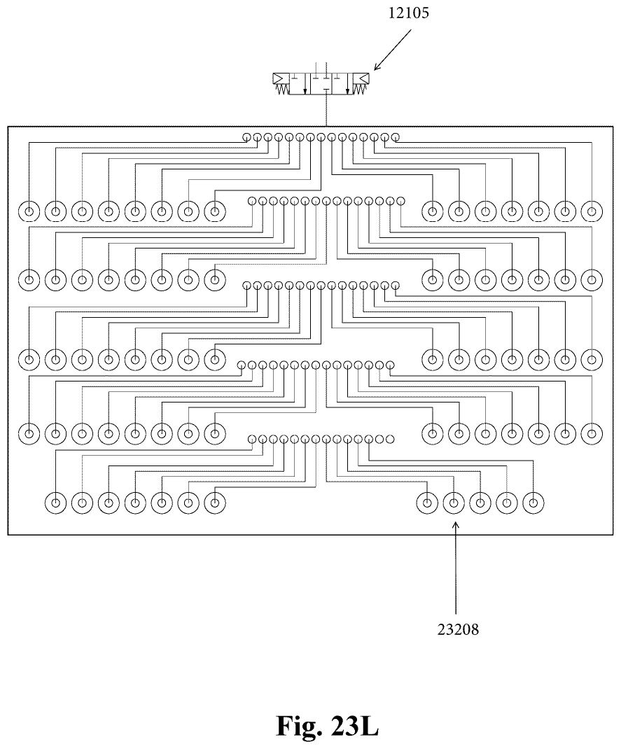

FIGS. 23A-K illustrate a perspective view of an embodiment of an assembled Human operating system 23100 in different views. In an embodiment, the assembled Human operating system 23100 consists of multiple interconnected subsystems. The subsystems are the mixing chamber 23103, the fluidic control operating system 23200, the human-on-chip plate 10104, the individual biochip component 100, the machine control system 23102, and the analytical device system 23105, 23110, 23112.

The fluids, cells, and other biological components leaving the system could be sampled and aliquoted at any designed time-lapses, by pumping, opening and closing their respective valves. Also, the tubes coming out of the system are interfaced with various measurement techniques, such as Fourier-Transform Infrared (FTIR) Spectroscopy devices, O2 & CO2 sensing devices, and pH sensing devices placed along the tubes coming out of the system.

In an embodiment, the human-on-chip plate includes a manifold that ensures the internal space of the system remains at specific environmental conditions that can be modulated for specific testing protocols. Additionally, heat dissipaters or heat generators may be utilized to mitigate undesirable temperature conditions.

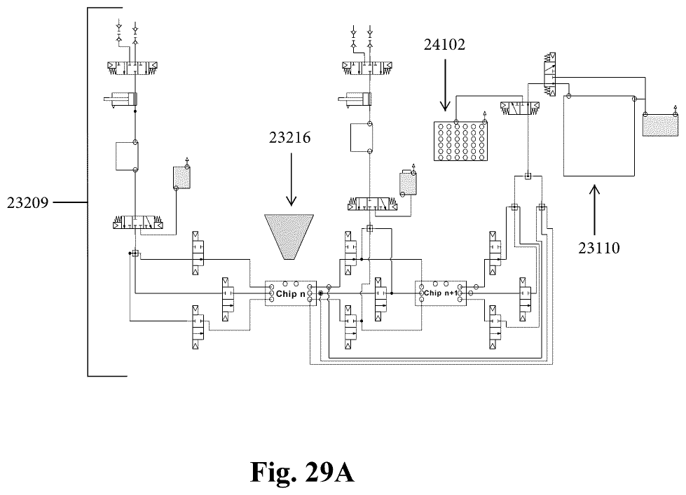

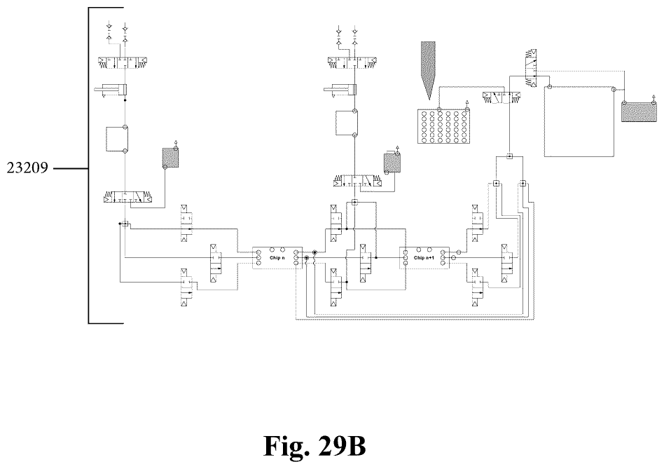

In an embodiment, the human-on-chip plate includes a fluid control system which is able to deliver and/or collect the necessary reagents, at precise times and channels of one or more biochips within the operating system, required to carry out analytical assays as shown in FIGS. 29A and 29B. These assays may be measured with the inbuilt microscope, spectrometer and/or FTIR, or with any other appropriate analytical device either inbuilt or not.

In an embodiment, the human-on-chip plate includes a fluid control system which is able to deliver and/or collect liquid samples from any channel in any biochip within the operating system. The liquid samples collected or delivered may contain any required chemical or biological material, which includes but is not limited to cells, enzymes, drugs, growth factors, nutrients, etc. The volume of the sample delivered to each channel can be between 0.5 and 25 μL.

In an embodiment, the human-on-chip plate includes a fluid control system which is able to pump liquid samples from any channel in any biochip within the operating system. These may be pumped either from the top inlet holes or from the side. The fluids delivered to the plurality of the channels within each biochip and/or across different biochips within the operating system may be the same or different. Gasses may also be pumped in one or a plurality of channels instead of a fluid. Liquids or gasses may also be pumped at different flow rates. Liquids being delivered may contain chemicals, drugs, polymers, biological materials, cells, growth factors, nutrients, or any other substance.

In an embodiment, the human-on-chip plate includes a fluid control system which is able to deliver a polymerizing substance which may contain cells or other biological or chemical substances to any or multiple channels (stromal, and/or ductal) of one or more biochips within the operating system. This control system may be coupled with temperature controllers to ensure that the temperature is maintained such that the polymerizing substance is in its liquid state until it is delivered to the desired duct. Once it has been delivered, the temperature will be raised to ensure gelification.

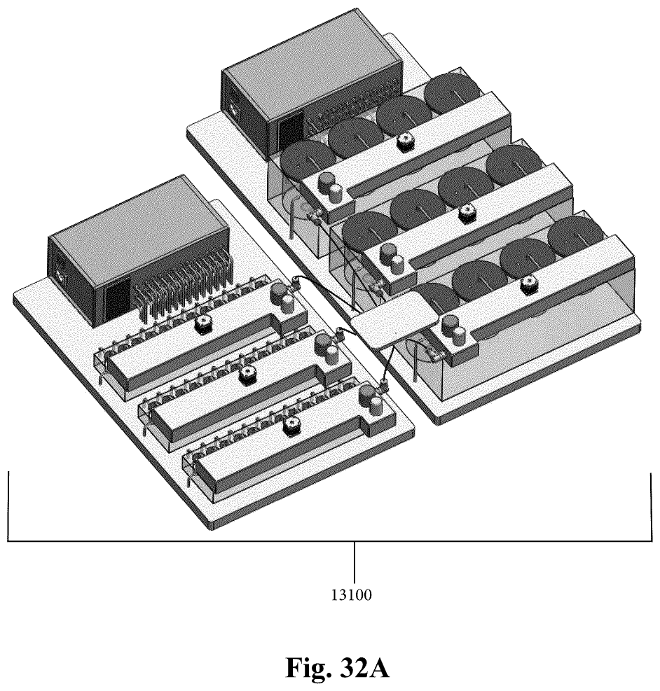

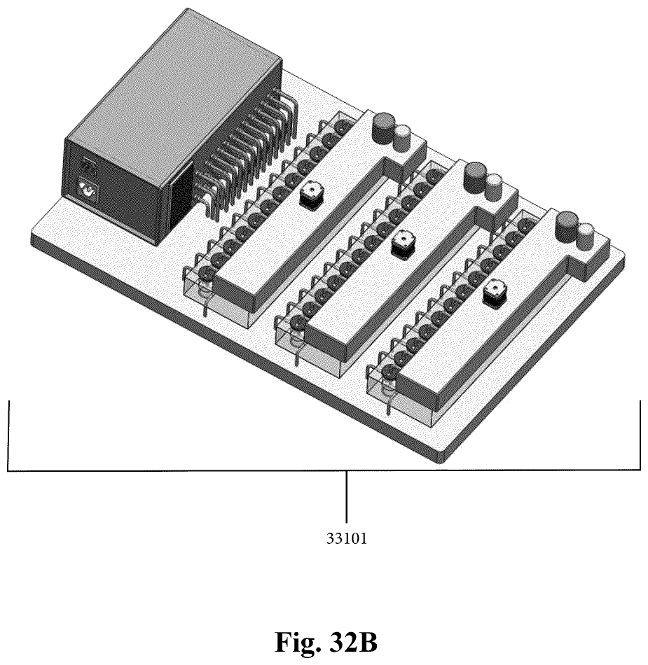

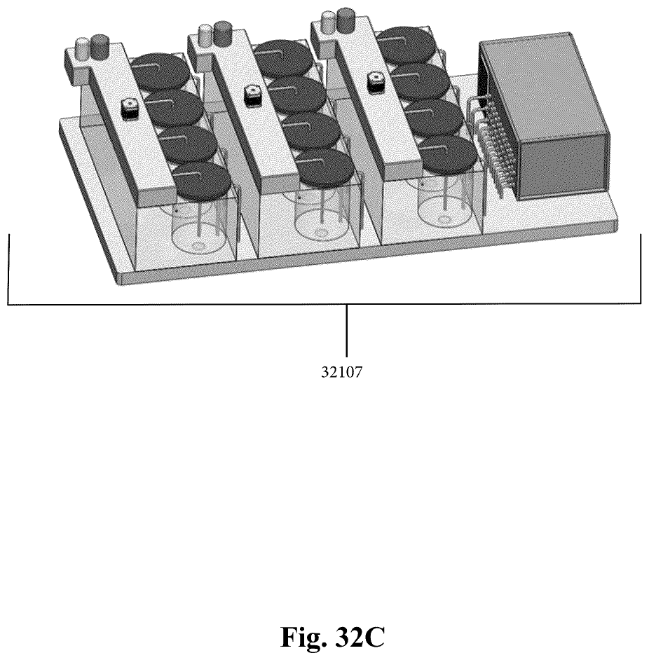

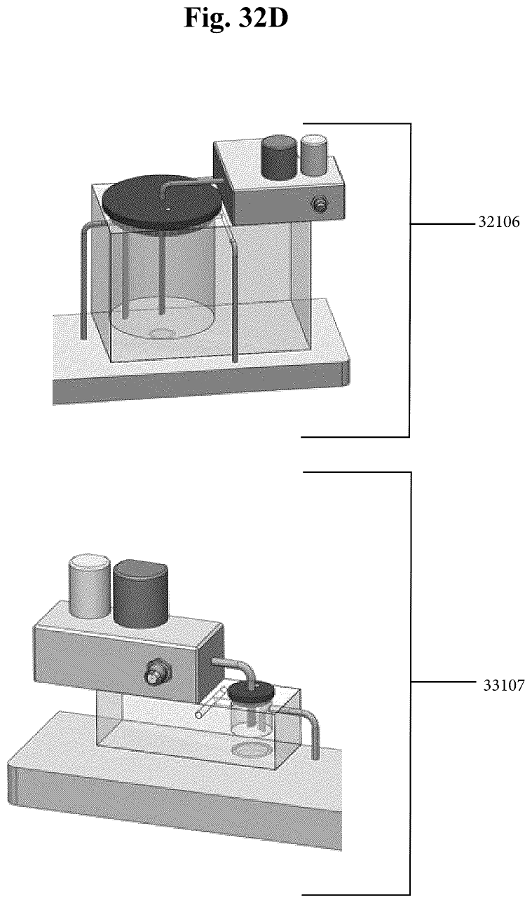

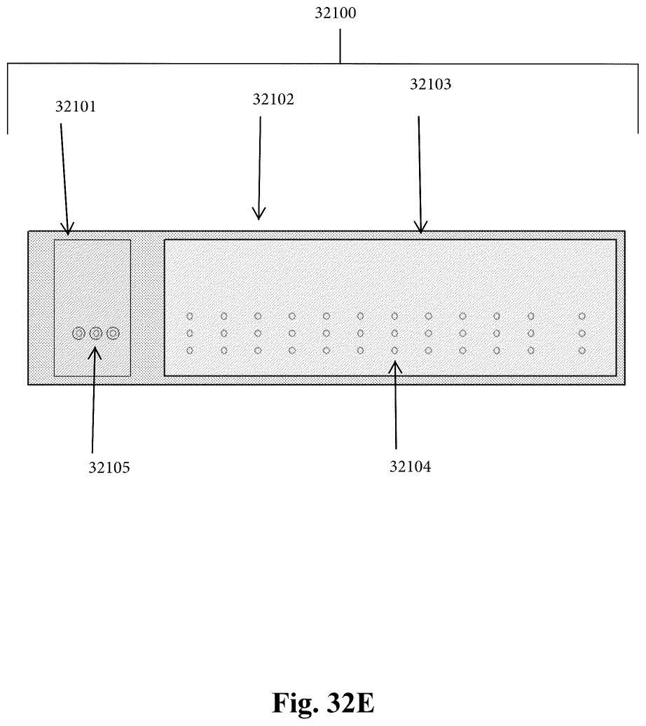

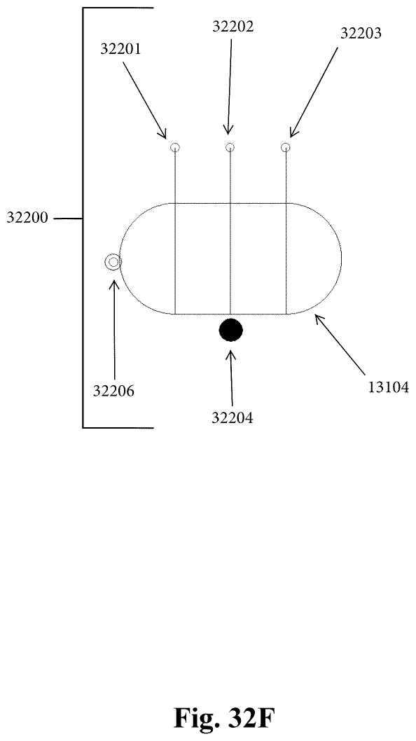

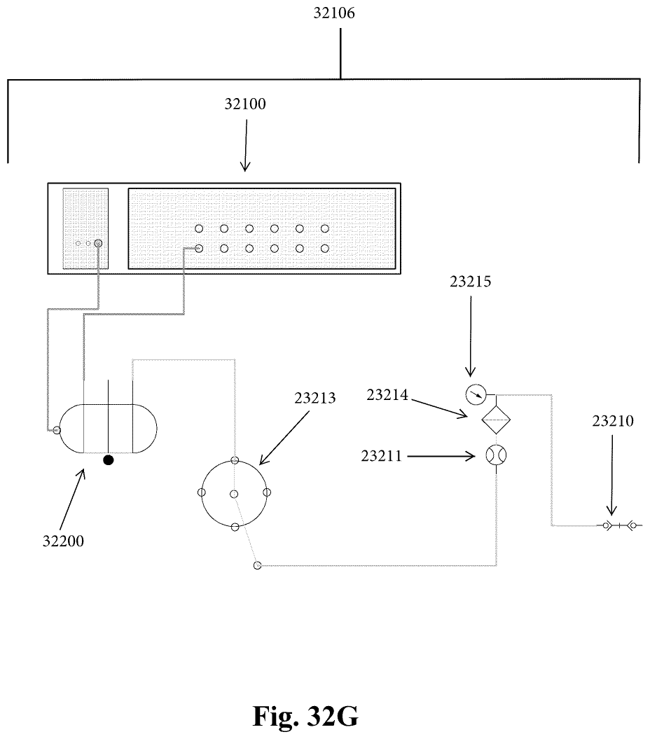

In an embodiment of the assembled human operating system 23100, the assembled human operating system 23100 is a benchtop analytic operation device that consists of multiple interconnected subsystems. This system allows the tissue culture of different organs within different arrangements, to mimic a human body. The microfluidic system designed in the assembled human operating system 23100, provide the ability to exchange any fluids in the system. FIG. 23C shows the assembled human operating system 23100 main components apart. The assembled human operating system 23100 has a cover 23101 to preserve the subsystems inside from any outside intervention, and a machine frame 23109 that holds all the different components together. The assembled human operating system 23100 is equipped with an electrical enclosure 23102 that powers the entire system 23100. Biochips 100 are assembled on the human-on-chip plate 10104 which is installed in the system using a sliding mechanism 23111. A three-axis robot 21100 can move freely in 3-dimensional space above any installed biochip 100. The system is equipped with multiple measuring and sensing devices that are fixed on the three-axis robot 21100, the measurement and sensing devices used include, but are not limited to, a spectrophotometer 23105, oxygen and carbon dioxide sensor 23106, and a microscope 23112. The spectrophotometer 23105 uses biological assays to measure the cell's viability and functionality inside the biochips 100. The O2 and CO2 sensor 23106 is used to monitor the dissolved gas concentration to maintain favorable conditions for cell seeding and proliferation or to measure induced hypoxic conditions. The microscope 23112 is used to examine and analyze the morphology of the cells inside the biochips 100. The fluidic operation system 23107 is responsible to feed and exchange fluids inside the system using the microfluidic channels. Media storage compartment 23108 is where the media will be discarded after its being used. ATR-FTIR 23110 is a measuring device to measure the molecule's composition in a media sample. The mixing table 23103 contains the mixing chamber 13100 described in detail in FIG. 32A, it will house the cells and media that will be used by the human operating system 23100.

FIG. 23F shows one side of the fluidic operating system 23200 including the ports that connect the human operating system 23100 with the tubes that moves the waste fluid out of the biochip 100 into the waste chamber, through the waste chamber ports 23114. This waste fluid is controlled by pneumatically actuated valves via the inlets 23113. Inlet ports 23116 connect the coolant or heated fluid between the fluidic operating system 23200 to the heat exchanger 25101 (shown in later figures) located above the human-on-chip plate. It also shows the ports that connect the Human operating system 23100 with the pneumatic air signal inlet 23115 that would actuate the valves allowing the Heat controlled fluid to pass via the inlets 23116. The Human operating system 23100 is equipped with media microchambers 23208, that store media in a close proximity to the biochips 100. Each micromedia chamber stores media and culture components that feed into individual microchannels within each biochip 100. The media microchambers 23208 are connected to the fluidic operating system 23200 through the media microchambers inlet ports 23118. The valves controlling the seventy-two media microchambers 23208 are actuated via sixteen multiplexed signals through the pneumatic air ports 23117 as shown in FIG. 23L. The thirty-three valves connecting the adjacent biochip microchannels within the human-on-chip plate 10104 are pneumatically actuated by eleven multiplex signals through the pneumatic air ports 23119.

FIG. 23G shows the side of the fluidic operating 23200 system containing four ports that control different sensors located on the human-on-chip plate 10104. Temperature sensors are controlled by electric signals coming from the electrical enclosure 23102 through the temperature sensors port 23201. Flow and pressure sensors relay a feedback signal to the electrical enclosure 23102 through the flow and pressure sensors port 23204. O2 sensors relay a feedback signal to the electrical enclosure 23102 through the O2 sensors port 23202, and CO2 sensors relay a feedback signal to the electrical enclosure 23102 through the CO2 sensors port 23203.

FIG. 23I shows how the human-on-chip plate 10104 connects to the fluidic operating system 23200, it shows the ports in the fluidic operating system 23200 that connects the biological fluids port 23207 to each channel of the human-on-chip plate 10104, also the pneumatic air control ports that connect with the valves 23205 inside the human-on-chip plate and the heated fluid ports 23206 that heats the human-on-chip plate 10104.

FIG. 23J shows the three main components in the fluidic operating system, which are the fluidic operating system device 23200, the media micro chambers 23208, and the human-on-chip plate 10104.

In an embodiment, the fluid consists of cells, and biological or chemical components, where media is first inserted manually by the user into the mixing chambers 13100. After being in the mixing chamber 13100, the media is pumped through a pressure sensor and an air bubble filter, and a 1-to-12-way valve 23212 into the tubings of the fluidic operating system 23200.

The 1-to-12-way valve 23212 allows for the media to pass from a predefined mixing chamber to the operating system before being distributed by the valving system, which is the first sub-system in the fluidic operating system 23200. The distribution valves consist of a combination of valves: one 1-to-12-way valve 23121 connected to 12 other 1-to-6-way valves 12105 creating a 1-72 routing pathway

FIG. 23L shows a schematic diagram of the media micro chamber 23208 that's located in close proximity with respect to the human-on-chip 10140, decreasing the distance between the 2 different components allows the use of a few microliters of fluid eliminating the possibility of diluting the samples. A 3/3 spring-loaded pneumatically actuated valve controls the flow of fluid from the mixing chamber 13100 into the media microchamber 23208. The fluid will travel into the 72-channel manifold. Each channel in the manifold leads to a single media micro-chamber 23208 that's equipped with an open-close gait allowing or restricting the fluid flow into the media microchamber 23208.

In an embodiment, the microfluidic pumping mechanism controls the precise amount of fluid which can be as little as 0.1 μL which is being pumped to the micro-chamber 23208 as well as from the microchamber to the biochips 100.

In an embodiment, each channel of a biochip has an inlet and an outlet port and the pumping control mechanism can control the pressure, flow and flow direction.

In an embodiment, at least one analytical device can be used for real-time measurement of chemical, biological, or physical parameters.

In an embodiment, the operating system contains pressure sensors at each biochip inlet and outlet fluid ports to manipulate the pressure inside the biochips' channels. This allows for the fluid to controllably perfuse from the ductal channel to the stromal one or vice versa.

In an embodiment of the operating system, at least one valving system could open or close and shift the flow pathways to every compartment, while the experiments are running.

In an embodiment of the operating system, the ductal scaffolds of a plurality of organ-on-chips on a human-on-chip plate are connected together allowing for a continuous flow.

In an embodiment of the operating system, the ductal scaffolds of a plurality of organ-on-chips on a human-on-chip plate are connected together only for a controllable amount of time.

In an embodiment of the operating system, the plurality of organ-on-chips on a human-on-chip plate are isolated from one another.

In an embodiment of the operating system, a microfluidic automated flow control mechanism allows for the addition and removal of fluid in the ductal scaffold of an organ-on-chip.

In an embodiment of the operating system a microfluidic, automated flow control mechanism allows for the addition and removal of fluid in the ductal scaffold of a plurality of organ-on biochips on a human-on-chip plate.

In an embodiment of the operating system, a microfluidic automated flow control mechanism allows for the addition and removal of fluid to the external compartment surrounding the ductal scaffold of an organ-on biochip.

In an embodiment of the operating system the pumping control mechanism can transfer fluid being pumped to a waste chamber.

In an embodiment of the operating system the pumping control mechanism can transfer fluid being pumped to a sample chamber.

In an embodiment of the operating system the pumping control mechanism can transfer fluid being pumped to a different biochip.

In an embodiment of the operating system wherein the pumping control mechanism can transfer fluid being pumped for sampling.

In an embodiment of the operating system wherein an electro-mechanically controlled robotic arm that is embedded and interlinked to the operating system, can operate in at least one dimension to perform different operations.

In an embodiment of the operating system an electro-mechanically controlled robotic arm that is embedded and interlinked to the operating system, can situate at least a built-in microscope, spectroscope or monitoring sensor over the human-on-chip.

In an embodiment of the operating system wherein an electro-mechanically controlled robotic arm that is embedded and interlinked to the operating system, can have at least one built-in pipette that can take samples from at least one compartment of the sample sourcing chamber.

In an embodiment of the operating system wherein an electro-mechanically controlled robotic arm that is embedded and interlinked to the operating system, can have at least one socket head that can engage and actuate at least one type of a mechanically actuated valve.

In an embodiment of the operating system wherein an electro-mechanically controlled robotic arm that is embedded and interlinked to the operating system, can have at least one built-in pipette that can take samples from at least one sampling spot 10116 on the human-on-chip plate 10104

### The Operating System Fluidic Control Mechanism

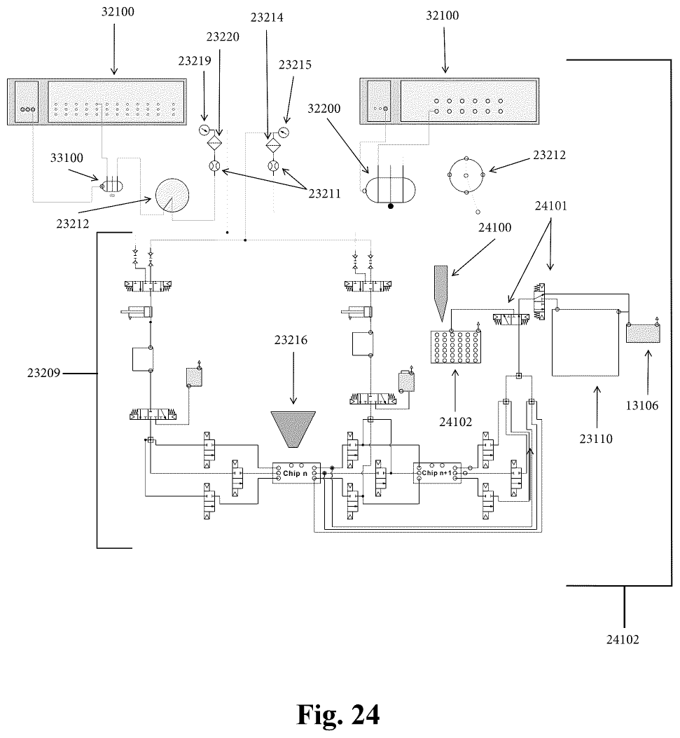

In an embodiment, a fluidic control mechanism allows the usage and manipulation of microliters of fluid to be used in the biochip cell culture, without losing milliliters of fluids in the process of reaching the biochips 100 channels as lost volumes in the tubings as shown in FIGS. 23K and 24. The close proximity to the biochip channels, the valving mechanism, and the small individual media chamber for every inlet and outlet hole play an important role in this system. In an embodiment, the piston pumping mechanism to insert the fluid into the individual media chamber and pump it to the biochips.

FIG. 23K shows a schematic diagram of the entire Human-operating-system 23100. The mixing chamber 13100 is divided into 2 main compartments that handle the cells and media. For the cells, the pressure unit 32100 is connected to cell chamber 33100. The pressure unit will provide the cells with the necessary gases for cell survival and move the cells from the cell chamber to the human-on-chip plate 10104. The cells will pass through a 12/1-way valve 23212 into a flow meter 23211, air trap 23220, and pressure sensor 23219 that will monitor and report to the system. For the media, the pressure unit 32100 is connected to the media chamber 33200 which will be connected to a 4/1-way valve 23213. The media will be monitored by a flow meter 23211, air tap 2314, and pressure sensor 23215. Both cells and media will pass through the quick connect 23210 into a 3/3 spring-loaded pneumatically actuated valve 12105 that will regulate cell and media flow into the biochip 100. In the Human operating system assembly 23209 a pneumatically actuated piston 12103 will pump the mixture into a media chamber located in close proximity to the biochip 100. After that, the system can control the mixture direction using a 3/3 spring-loaded pneumatically actuated valve 12105 that can direct the mixture either to a waste chamber 13106 or to a set of 3 2/2 bistable pneumatically actuated valve 13102 that operated in a pre-determined logic that allows the mixture to be sent to the first duct 102, second duct 103, stroma 103 or all three sites at once.

FIG. 24 shows a schematic diagram of the entire Human-operating-system 23100 with the Human operating system assembly 23209 containing the measuring devices. The measuring devices are a microscope/spectroscope/O2 and CO2 assembly 23216 and an ATR-FTIR unit 23110. Human operating system assembly 23209 is equipped with a sampling site 24102 and a 3-axis robotic external sampling pipette 24100 that can be used to extract samples from the system. To control the flow of the sample a set of 3/2 spring-loaded pneumatically actuated valves 24101 can direct the flow to the sampling site 24102, ATR-FTIR 23110, or a waste chamber 13106. This assembly represents an embodiment of the operating system flow diagram 24102.

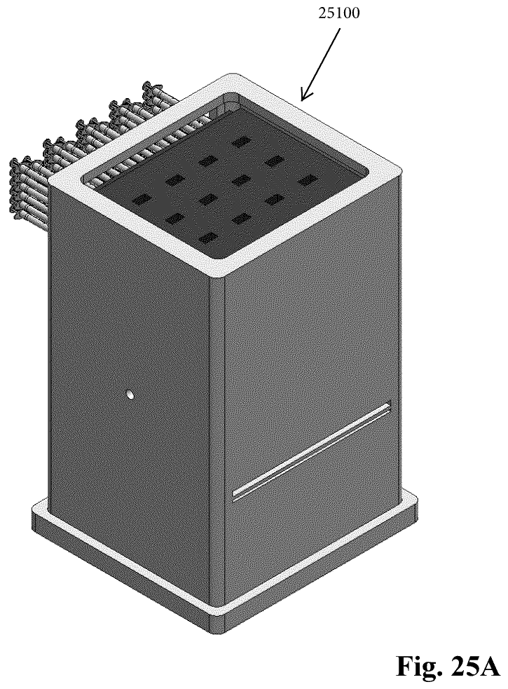

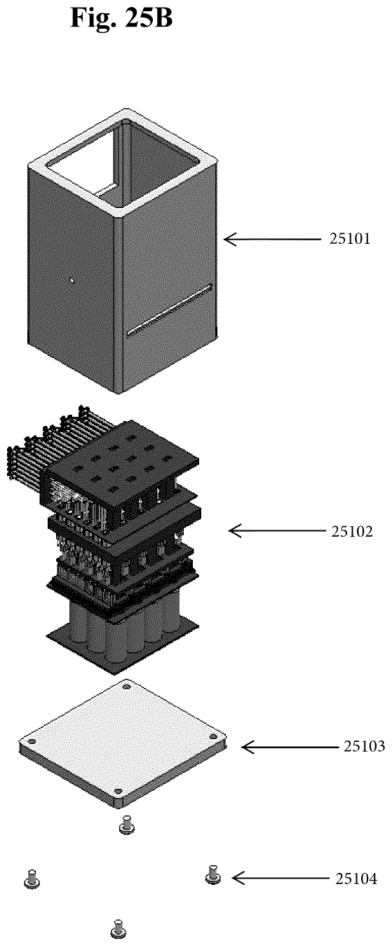

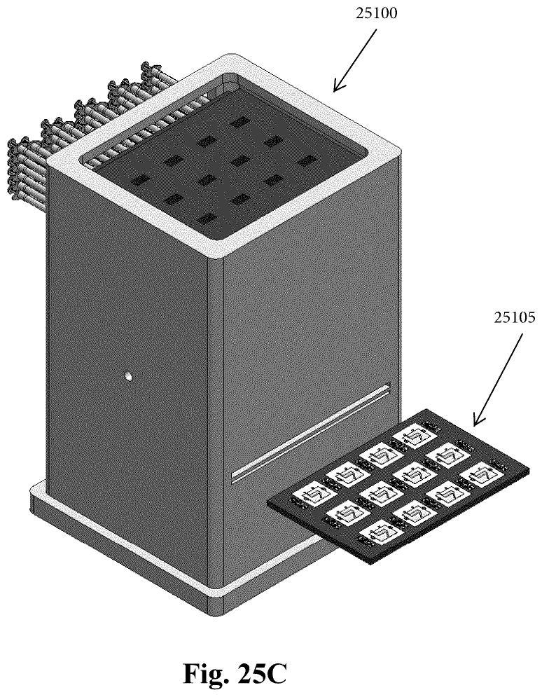

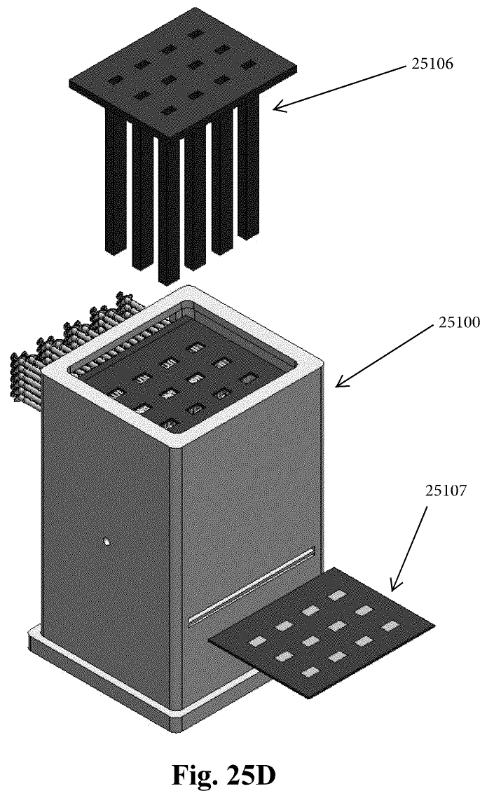

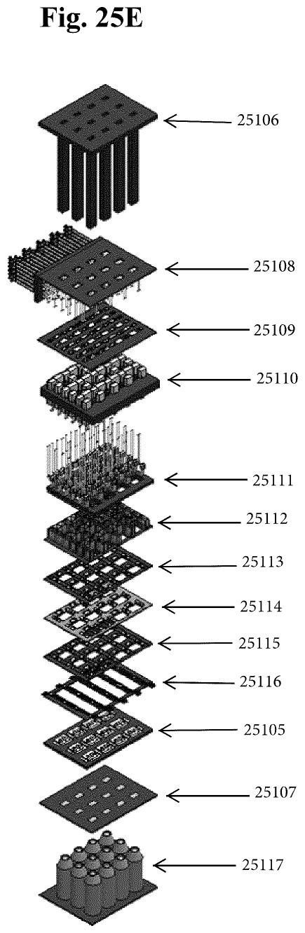

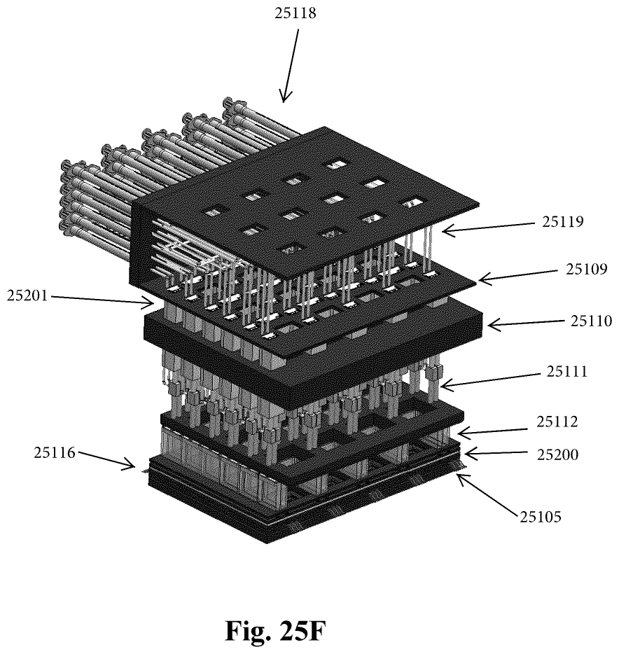

FIGS. 25A-E show an embodiment of the Human operating system 25100, where the system components are stacked over each other in layers 25102. The assembly is covered by a protective cover 25101 with a base plate 25103 that's held in place using bolts 25104. In this embodiment, the human-on-chip plate 25105 will be fed into the system from the side and the spectroscope sensing plate 25107 is located under the human-on-chip plate 25105 with the spectroscopy source plate 25106 on top. This embodiment of the human operating system consists of 12 stacked layers 25102 with each layer providing a certain set of functionalities, the spectroscope source plate 25106 situated on top of the device is used as a measuring device to monitor cell functionality. Underneath that, the top media sourcing plugs plate 25108 that stores media and a top open-close valve plate 25109 controls the media flow in this embodiment of the human operating system 25100. The piston actuator plate 25110 and piston plate 25111 pump media from the top media source plugs plate 25108 into the micro media chamber plate 25112. After that, the media will go through the upper open/close valve plate 25113, middle direction shift valve plate 25114, and lower open/close valve plate 25115 that change the direction of the media sending it to a specific biochip 100. An inlet/outlet valve actuator plate 25116 will be the final media control point that allows it to flow into/out of the biochip 100 that's located on the human-on-chip plate 25105. The spectroscope sensing plate 25107 will receive the emitted light from spectroscopy source plate 25106 through the biochip 100 found on the human-on-chip plate 25105. Spectroscopy source plate 25106 can move aside allowing the microscopic array plate 25117 to have a clear line of sight to perform microscopic imaging of the cells culture in the biochip 100.

FIG. 25F shows the fluid pumping control system where a syringe pump 25118 is connected to the top open/close valve plate 25109 via connection tubing 25119. At this stage, the fluid pumping control system controls the amount of media that will be available to be used in the cell culturing stage. Primarily the media will be stored in the media storage, using the syringe pumps 25118 we can withdraw the media and transfer it to the micro media chamber plate 25112 that is placed on top of the human-on-chip plate 25105. Using such an arrangement the media will be situated directly on top of the cultured cells in the biochips 100 of the human-on-chip plate 25105. Having the media in such proximity will shorten the distance and decrease the possible variability that might affect the media. To control such a small amount of media, a set of pistons 27102 are fixed on a piston plate 25111 sandwiched between a piston actuator plate 25110 and the micro media chamber plate 25112. The piston actuating plate 25110 will control the pistons 27102 extensions and retraction movement allowing a small amount of media to flow into the micro media chamber plate 25112. In between the micro media chamber plate 25112 and human-on-chip plate 25105 a valving plate 25200 is placed, this plate works alongside the inlet/outlet valve actuator plate 25116 to control the fluid flow in and out of the Human-on-chip plate 25105.

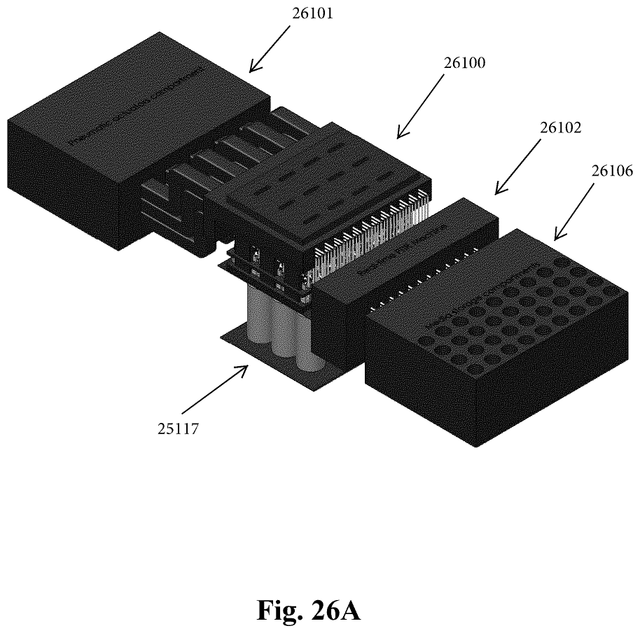

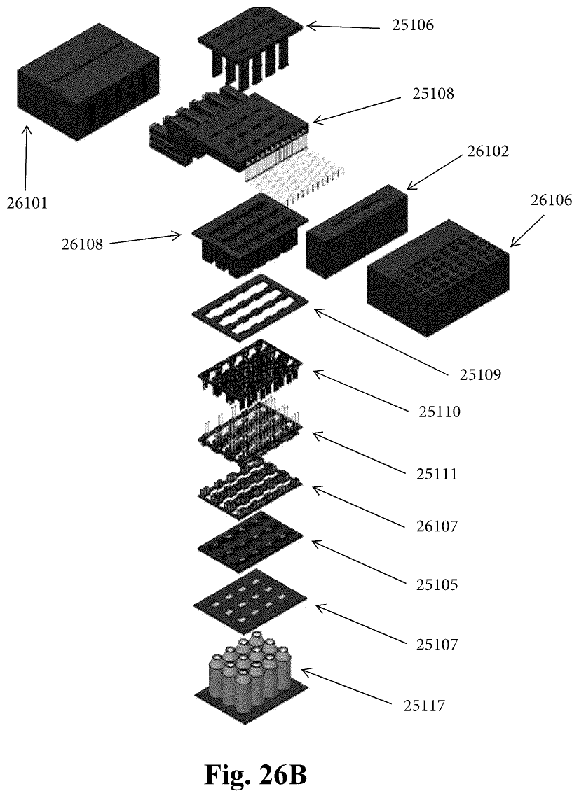

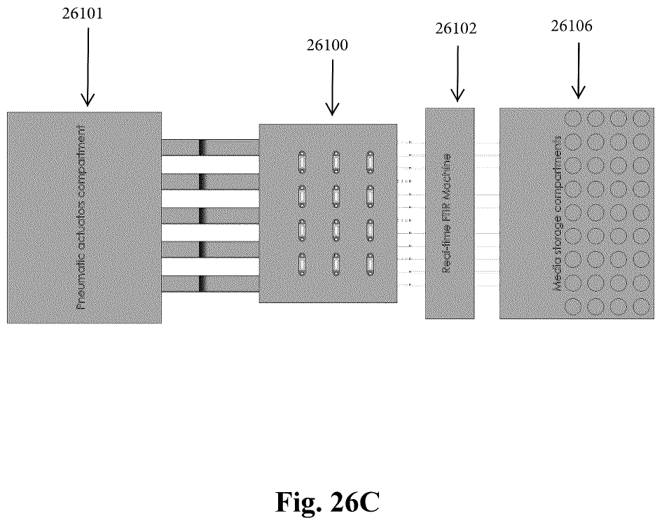

FIGS. 26A-C show another embodiment of the Human Operating System 26100 wherein the system is equipped with a pneumatic actuator container 26101, FTIR machine 26102, media storage container 26106, and microscope array 25117. The integrations of all the previously mentioned components increase the versatility and usability of the machine. The same stacking concept embodiment as in FIG. 25A-F may be employed in this embodiment with some modifications. A pneumatic actuators container 26101 can be used to control multiple components of the system wherein the pneumatic actuators container can be used as a pressure source to drive media from the media storage container 26106 into the micro media chamber plate 26107 passing through a top media source plugs plate 25108 that's designed to transfer few microliters of fluids. After the fluid quantity is reduced to a few microliters a set of highly modified pistons 27102 capable of handling such quantities fixed on a piston actuator plate 25110 that can be pneumatically controlled by the pneumatic actuators container 26101 linked to a pneumatic actuating valve fitting plate 26108 to derive media or other fluids from the media storage container 26106 in the micro media chamber plate 26107 that is situated on top of the human-on-chip plate 25105 that houses the biochips 100 where tissues will be cultured. This embodiment of the human operating system 26100 is equipped with a triple measuring system comprising a spectroscope sourcing plate 25106 emitting a signal to a spectroscope sensing plate 25107. The cultured tissues are also interlined with real-time FTIR 26102 using sampling channels where a fluid sample can be excreted to be tested. Both work together to gather data about the effect that the drug exerts on the tissues. A microscope array 25117 consisting of multiple microscope lenses is placed underneath the human-on-chip plate 25105. All three systems will be used to track the biological assays that will indicate the cell's morphology, viability, and drug efficacy.

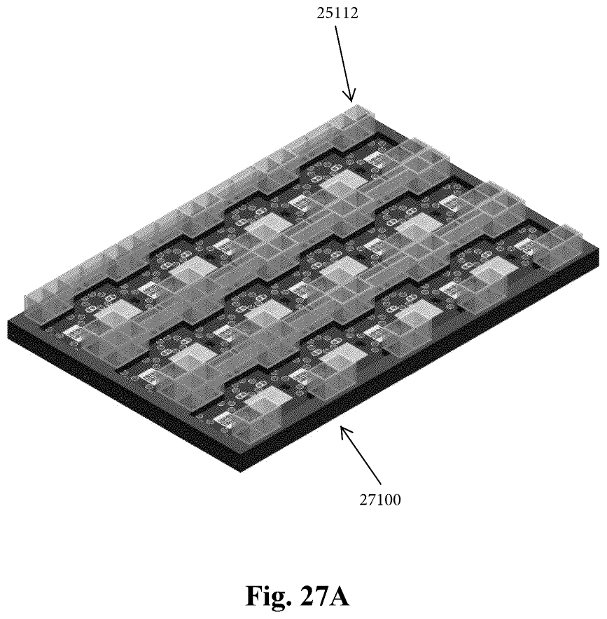

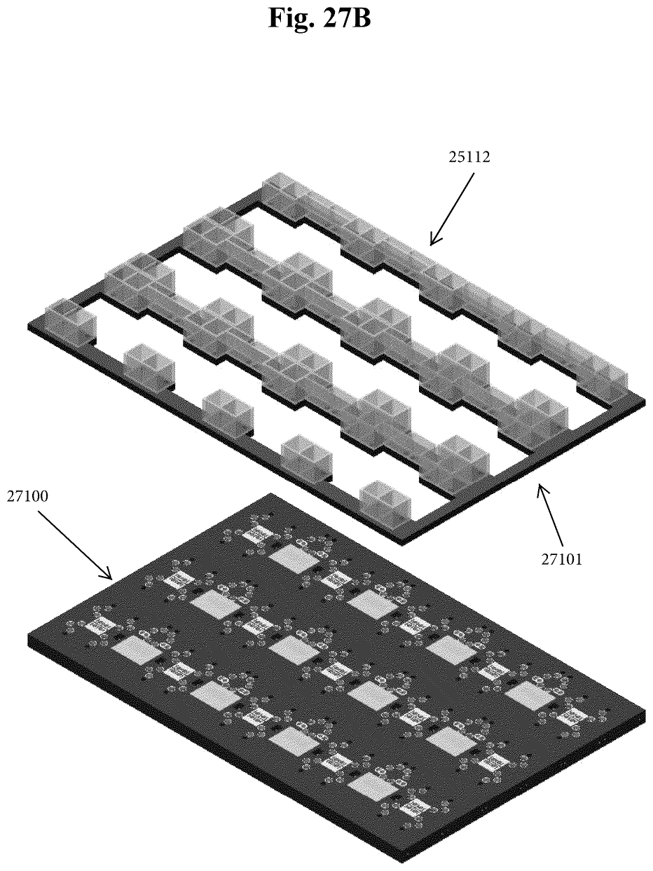

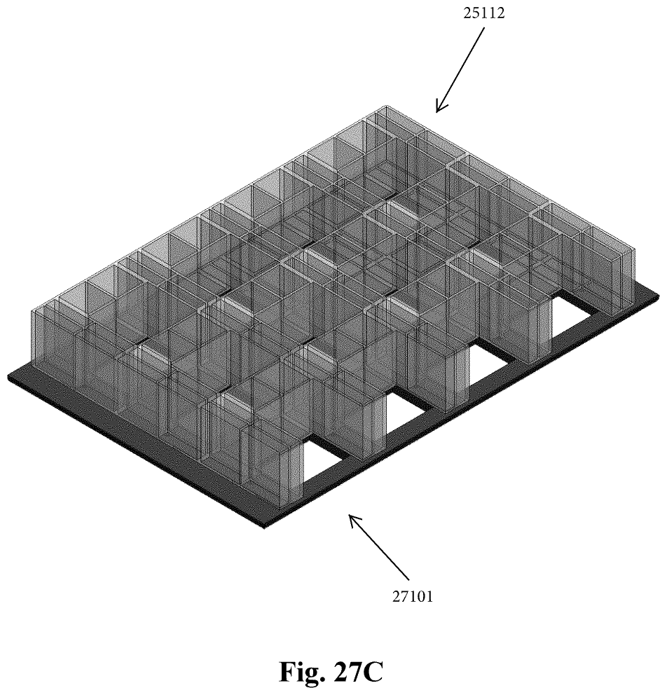

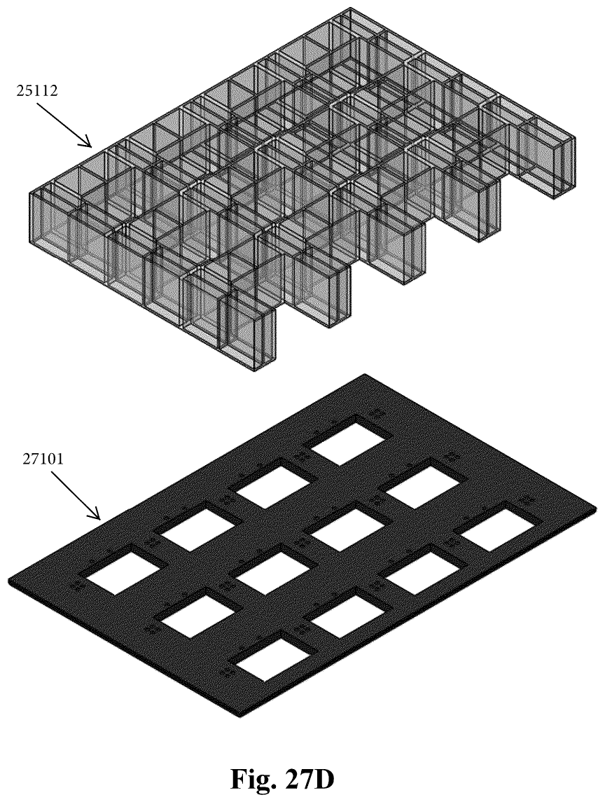

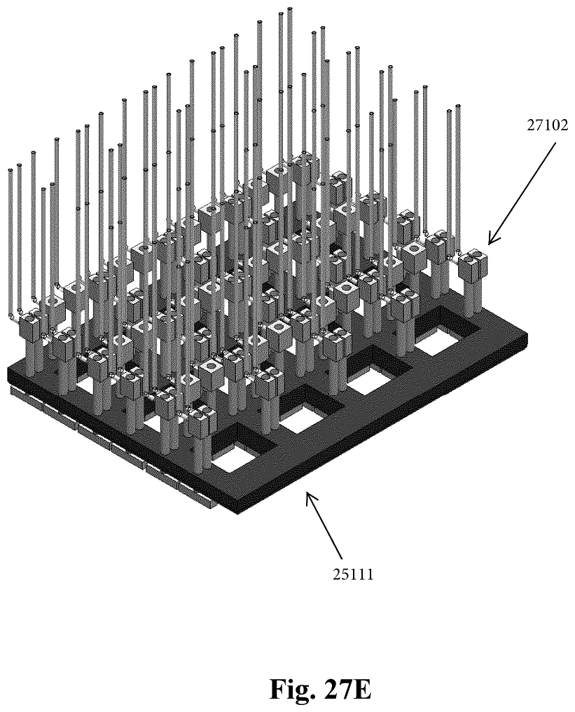

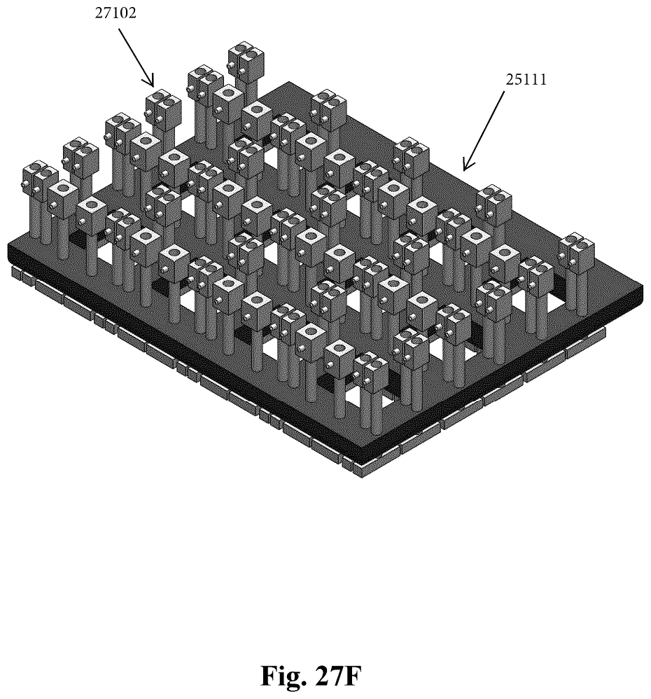

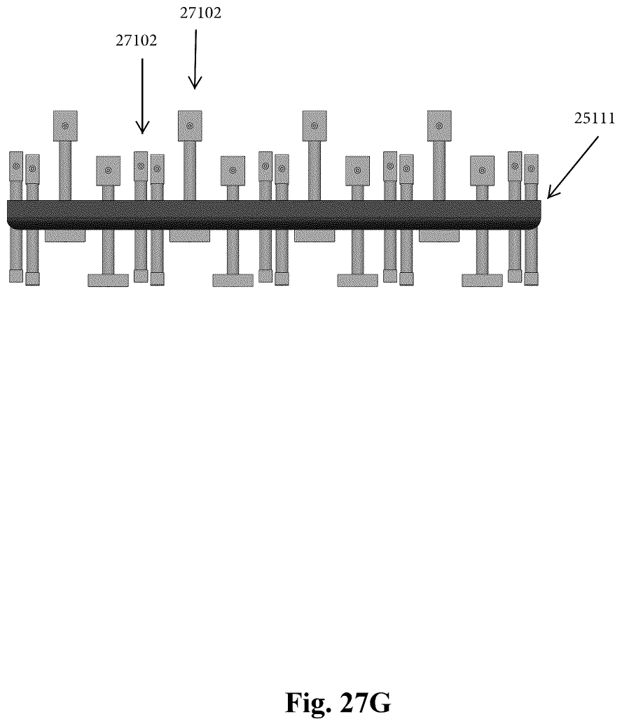

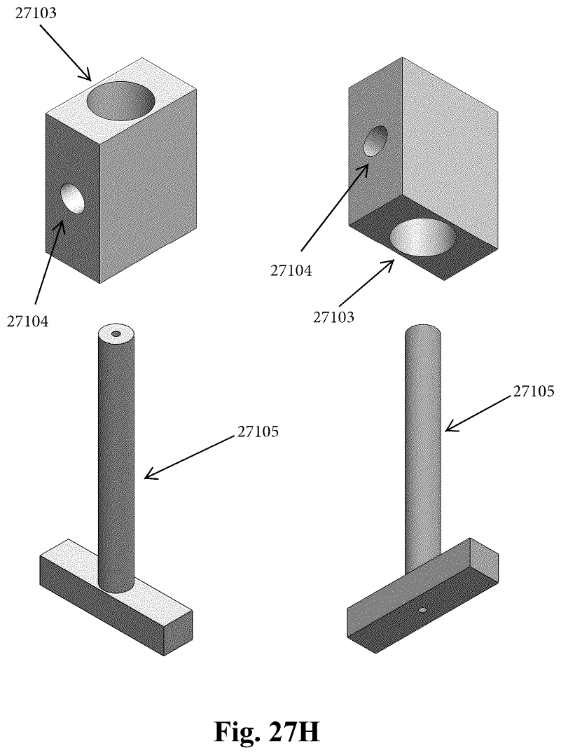

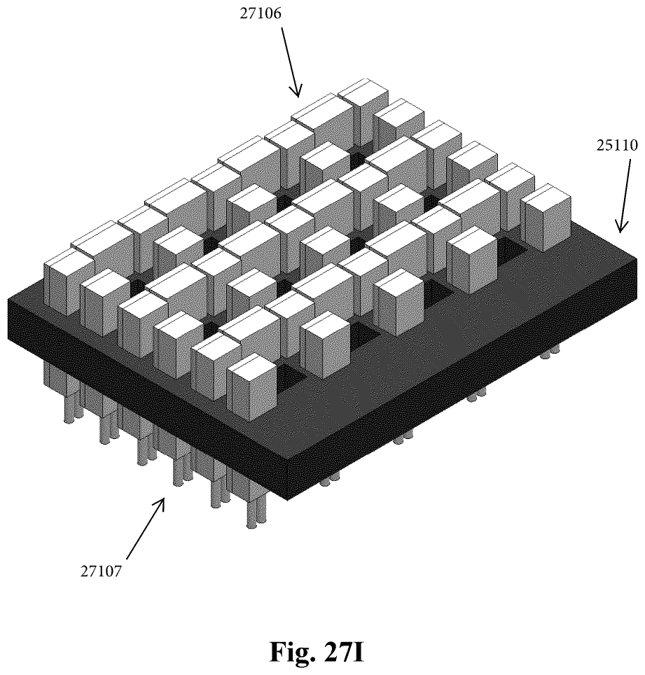

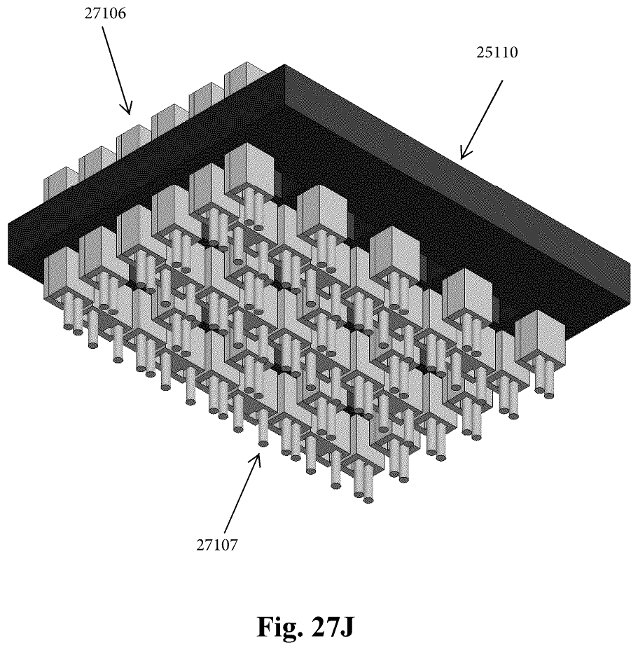

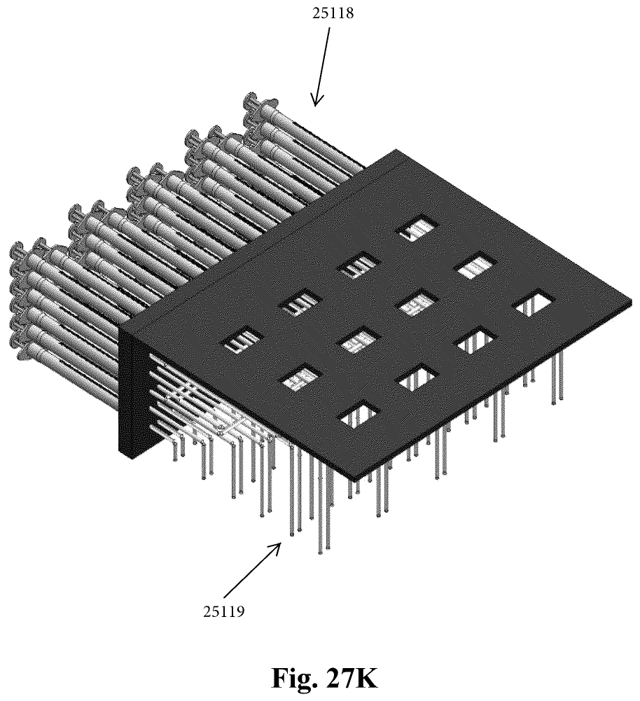

FIGS. 27A-D show a closer view of the micro media chamber 25112 placed on top of the human-on-chip plate 27100. Having the micro media chamber 25112 in such an arrangement will facilitate the maintenance of this machine since they are connected to a removable micro chamber media plate 27101. The main reason why this arrangement was used is to have a minimum distance between the fluid that will be used in the cell culture and the human-on-chip plate 27100. The close distance will eliminate the possibility of diluting the sample since the bulk of the fluid used will not need to travel for a long distance from the media storage container 26103 to the human-on-chip plate 27100. Moreover, the close proximity of the fluid stored in the micro media chamber plate 25112 to the human-on-chip plate 27100 will provide quick, accurate, and responsive feedback especially when drug cytotoxicity or efficacy is being tested since the drug will need to travel in a very small quantity for a very short distance without being diluted.

FIGS. 27E-G show the pistons 27102 and the piston plate 25111 that will be used to transfer the fluids from the media storage container 26106 into the micro media chamber 27101. The pistons will alternate between their extended and retracted position to move the fluid, these pistons 27102 can be actuated mechanically, fluidically, electrically, or pneumatically. In the side view of FIG. 27G, the different positions of pistons 27102 on the pistons plate 25111 can be seen.

FIG. 27H shows a detailed view of the pistons as represented. The piston shaft 27105 which can be mechanically, fluidically, electrically, or pneumatically actuated goes into a cylindrical cut-out 27103 that will receive the fluid from the media storage container 26106 through an inlet port on the side of the piston housing 27104. Once piston 27102 is in its retracted position the fluid is sucked into the cylindrical cut-out 27103 through the inlet port on the side of the piston housing 27104, after that the piston is actuated using one of the previously mentioned methods causing it to move to its retracted position covering the side inlet port and compressing the fluid forcing them to be transferred into the micro media chamber 25112.

At the interface with the media chamber plate is a piston plate that contains individually actuated pistons for every media chamber. Each piston in the piston plate is made of two subcomponents. The lower component of the piston has a bottom surface cross-section with the same size and shape as the micro-media chamber 25112 it operates. In addition, the piston contains a channel across its length that goes to the upper component of the piston. The upper component of the piston contains on its lower surface an interface with the lower piston component. This interface is open to a channel that connects the channel of the lower piston component open at the media chamber, to the side of the upper component of the piston where a tubing interface is located that takes the media from the micro-media chamber 25112 through the piston 27102 towards the top valving plate 25109. The full piston plate 25111 is assembled by inserting the lower piston components 27105 into the plate containing the holes for the pistons from below, and the upper piston components 27103 to their respective lower pistons 27105 from the other side. The top surface of the upper piston components contains a pressure plugging interface feature 27103 that is interfaced with a respective piston actuator 27106 contained in the piston actuator plate 25110.

FIGS. 27I-J show the actuators 27106 on piston actuator plate 25110. On the bottom side, the actuator shaft 27107 will be connected to the piston cylindrical cutout 27103 which actuates the pistons to their open and closed state by switching between the extended and retracted position of the actuator shaft 27107. At the interface with the piston plate 25111, is a piston actuator plate 25110 that contains an actuator for each piston. The actuator 27106 could be directly connected to the piston 27102, or through a connector that reaches the actuator located outside the system. The actuators included in the operating system 26100 interfaced with the piston plate 25111 as shown in FIGS. 27E-H are designed to be of small size, dissipate minimal heat, and move a small and controllable step (for example, squiggle motors). Other motors could be used in the system, such as solenoidal magnetic actuators or small stepper motors. For the actuators that are located outside the operating system, any actuator type could be used, as long as the interface is prone to minimal errors. The pneumatic or hydraulic actuators 26101 could be used to actuate the pistons from outside the system, as long as the tubing connecting the operating system external actuators to the system are thermally controlled, and sealed and the compressibility of the gasses is taken into account.

FIG. 27K shows the top media source plugs plate 25108 of the human operating system 26100, where in this embodiment the system is equipped with a set of syringe pumps 25118 that's connected to the pistons inlet port 27104 via connection tubes 25119. The syringe pumps can be replaced with other forms of fluid pumping like pressure-driven pumps.

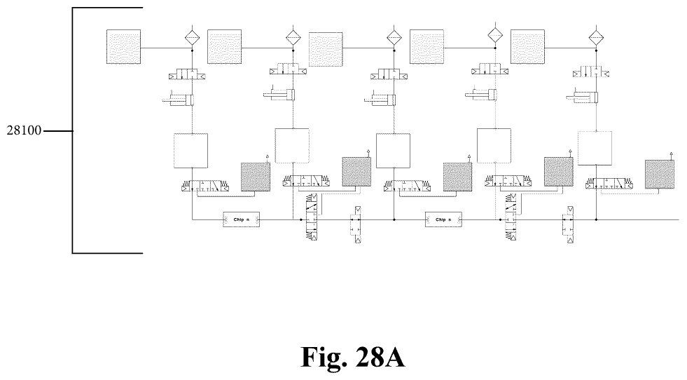

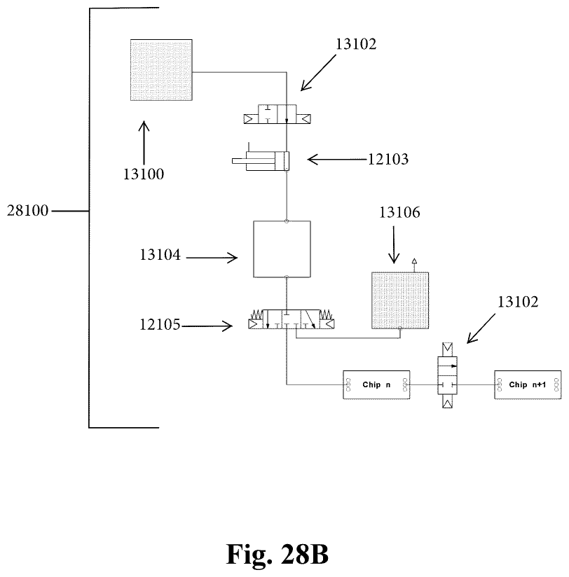

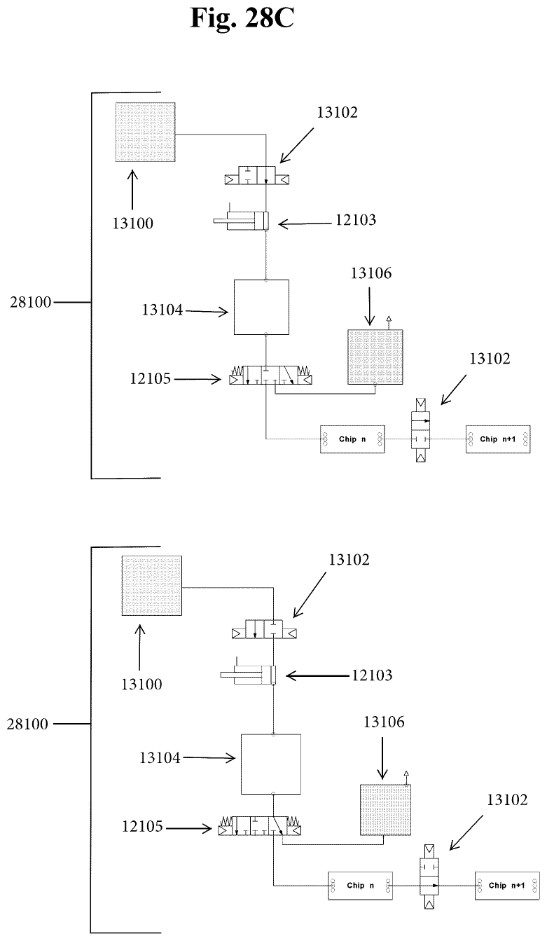

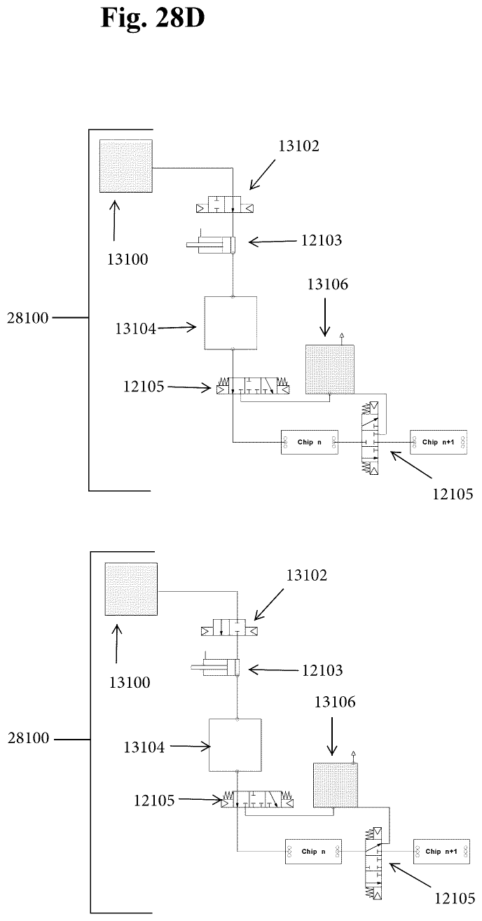

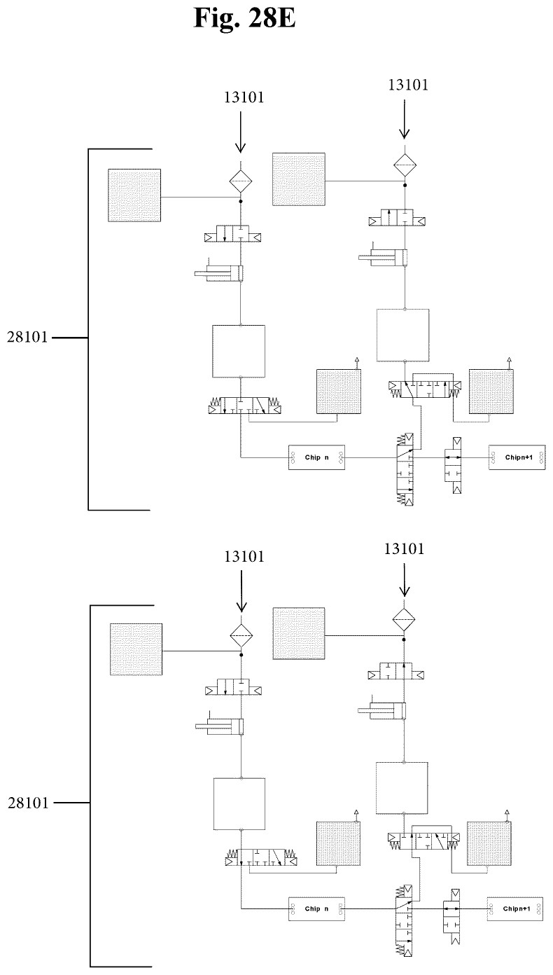

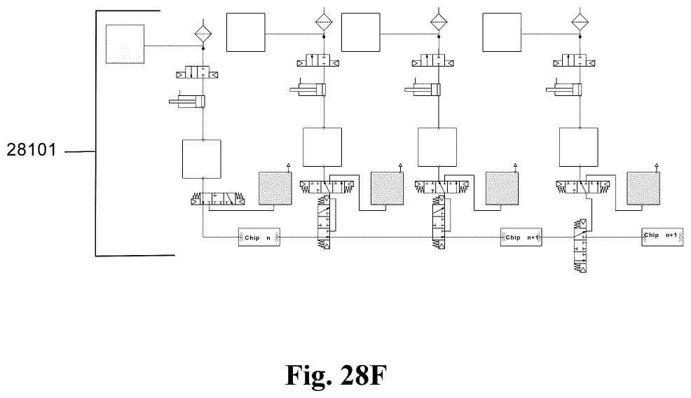

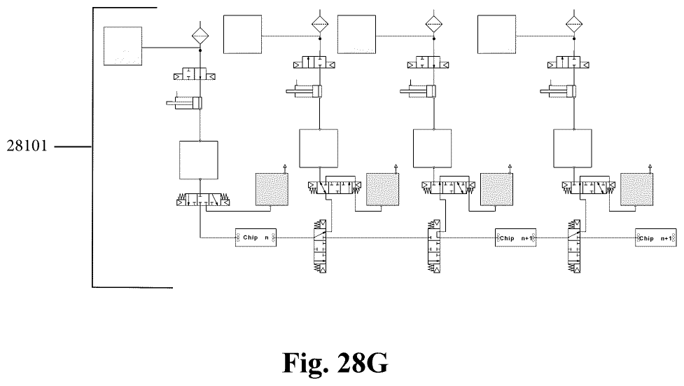

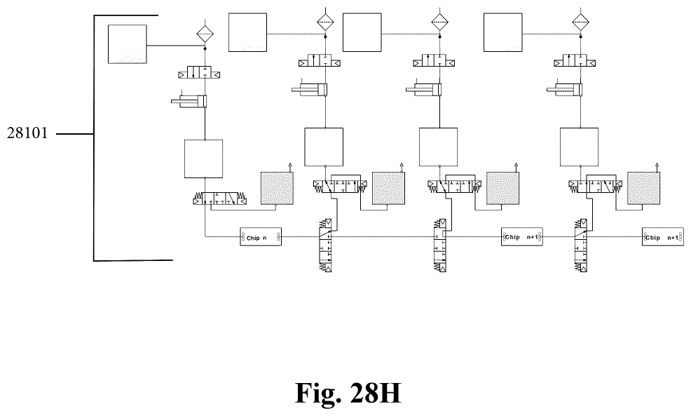

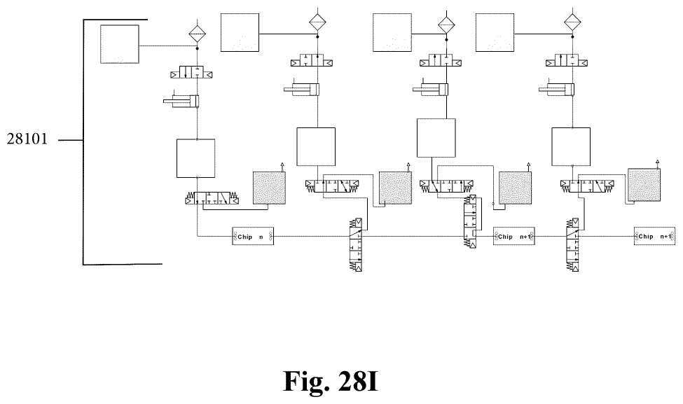

FIG. 28A shows a schematic view of the interface of the operating system with the biochip in the human-on-chip 28100 wherein the system has different levels of valves that can be actuated in multiple different ways separately. The first level of valves will control the media and other fluid flow into the media chamber, and the second level of valves control the fluid and media flow into the biochip or the waste chamber. The last level controls the fluid and media leaving the biochip, the media leaving the biochip can be induced into another single or multiple biochips wherein the valves can operate in different open/close positions driving the fluids into a different location.

FIG. 28B shows a schematic view of the initiation process of the human-on-chip 28100 where a sequence of preprogrammed instructions will test for any failure in the valve actuation, then the media or other fluids will be pumped from the mixing chamber 13100 into a 2/2 bistable pneumatically actuated valve 13102 that's switched into its open position allowing media to pass into a pneumatically actuated piston 12103 that will extend and retract to force the media into the media chamber 13104. This will be the priming stage that will follow the initiation process. A 3/3 spring-loaded pneumatically actuated valve 12105 will be in its closed position to prevent the fluid from escaping into a biochip 100 or a waste chamber 13106 while the media chamber 13104 is being filled.

FIG. 28C shows schematically the steps after the priming stage where the 2/2 bistable pneumatically actuated valve 13102 is switched into the close position preventing the media from being forced back into the mixing chamber 13100 by the pneumatically actuated piston 12103. In the next step the 3/3 spring-loaded pneumatically actuated valve 12105 will switch into the open position directing the fluid flow into the waste chamber 13106 then the pneumatically actuated piston 12103 will be actuated to push the fluid into the waste chamber. This step is to flush the system 28100 in preparation for cell seeding.

FIG. 28D shows a schematic of a human-on-chip 28100 after the flushing stage wherein the media was pumped into the media chamber 13104 from the mixing chamber 13100 through a 2/2 bistable pneumatically actuated valve 13102 by a pneumatically actuated piston 12103. In this embodiment, the 3/3 spring-loaded pneumatically actuated valve 12105 is in its open position directing the flow into the biochip. A 3/3 spring-loaded pneumatically actuated valve 12105 was placed on the biochip outlet to control the fluid coming out of the biochip 100, this valve can direct the fluid to the waste chambers 13106 or to another biochip 100 on the human-on-chip plate 10104.

FIG. 28E shows another schematic embodiment of the human-on-chip 28101 where the system is equipped with an air trap 13101. In this embodiment the system operates in the same stages as mentioned in FIGS. 28B-D, where the system will run the initiation sequence then the priming and flushing stage, and after that the cell seeding and media flow. In this embodiment, the human-on-chip flow diagram 28101 is equipped with an air trap 13101 that will allow the manipulation of gas content in the media without the formation of bubbles inside the system. Each biochip is connected to two different assemblies of the media chamber, valves, and waste chamber, such arrangement will allow the control of fluids going in/out, and the type of fluid going into the ducts 101, 102 and the stroma 103 or it will allow each biochip 100 to derive fluid from a separate media chamber through setting the 3/3 spring-loaded pneumatically actuated valve 12105 is a certain open/close position.

FIGS. 28F-28I show the human-on-chip flow diagram 28101 wherein multiple biochips 100 are connected to multiple media sources, all interlinked by a valving system. The human operating system will consist of the human-on-chip plate 10104 which will house the twelve different biochips capable of hosting multiple cell cultures. The cell culture can consist of a single cell type or can be a co-culture of at least one different cell type, seeding in the stroma and ducts of the biochip. A sample of fluid can be extracted from the biochip one and then transferred to a different biochip on the human-on-chip plate 10104.

FIGS. 29A-B show the human-on-chip plate flow diagram 23209 with the measuring and sensing devices. One feature of this Human operating system 23100 is the integration of multiple measuring and sensing devices directly into the system. The measuring devices include the microscope/spectroscope/O2/CO2 assembly 23216, and an ATR-FTIR 23110 alongside the sampling site 24102. Each measuring and sensing device is characterized by certain functionality that the system will rely on to monitor and maintain the cultured tissues and will measure the cell reaction, efficacy, or cytotoxicity of the introduced drug. The sampling site can be accessed manually by the technicians or automatically using the 3-axis robotic external sampling pipette 24100.

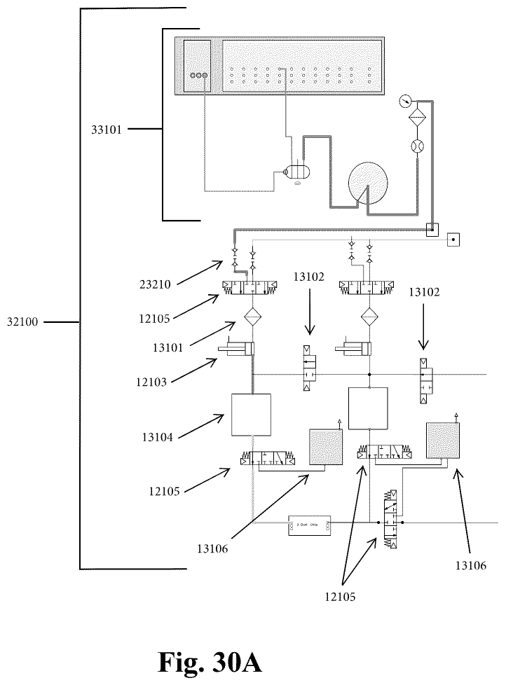

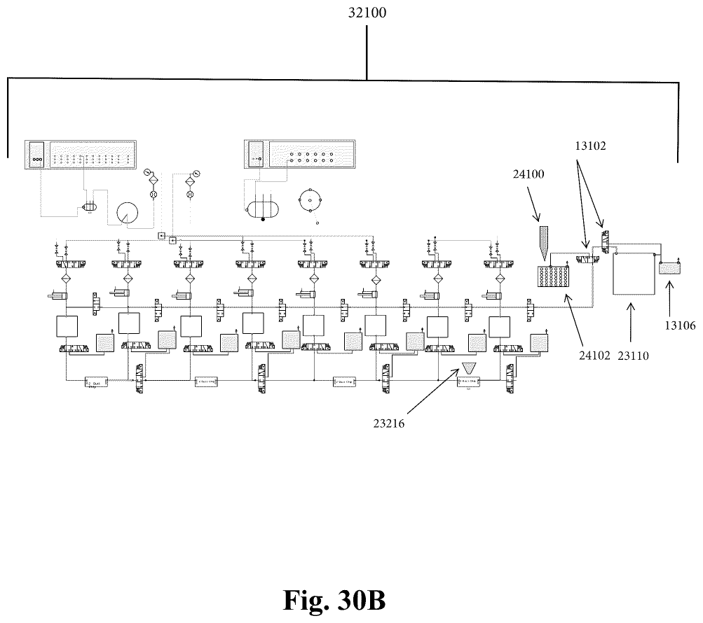

FIG. 30A shows schematically the operation of the Human operating system 32100 wherein the cells are extracted from the cell mixing chamber 33101 into the quick connect 23210 that connects the human-on-chip plate 10104 to the human operating system 23100. A 3/3 spring-loaded pneumatically actuated valve 12105 will have 3 major functions, first of which is to allow the flow of the cell into the human-on-chip plate 10104. After that the valve can switch into its second position allowing the media flow. And the third functionality will be in a closed position which will prevent anything from going in or out of the human-on-chip plate 10104. The third functionality of this valve is especially important since the human-on-chip plate 10104 is removable and since it is equipped with a pneumatically actuated piston 12103 that will force the fluid into the media chambers 13104 causing the fluid to go back to the mixing chamber 13100. An air trap 13101 mediates the 3/3 spring-loaded pneumatically actuated valve 12105 and the pneumatically actuated piston 12103. After that the fluid will flow into the media chamber 13104. In some cases, since this is an interlinked system a 2/2 bistable pneumatically actuated valve 13102 is connected to the channel in between the media chamber 13104 and the pneumatically actuated piston 12103. based on the position of the valve fluid can either flow to the media chamber 13104 or can travel to the next junction. this arrangement allows the sampling out of either a single biochip or from multiple biochips without the need for the sample to pass through all biochips found on the human-on-chip plate 10104. In this figure, the cells had passed through all the previously mentioned parts into the media chamber 13104 and then through the 3/3 spring-loaded pneumatically actuated valve 12105 located underneath the media chamber into the biochip. The 3/3 spring-loaded pneumatically actuated valve 12105 may be located underneath the media chamber and control the flow direction by either blocking the flow, diverting the flow to the waste chamber 13106 to be excreted out of the system or allowing the flow to go into the biochip for flushing, cell seeding, and refeeding.

FIG. 30B shows a full representation of the human operating system 32100 with four biochips wherein the valving system connects the four different biochips to each other and to the different components of the human operating system 23100. The measuring and sensing assembly that consists of microscope/spectroscope/O2 sensor/CO2 sensor assembly 23216 will monitor the cultured tissue before and after inducing the drugs. After that, the operator can choose to extract a sample where the different valves will switch into their open or closed state in which they will create a pathway for the sample to travel to reach a set of 2/2 bistable pneumatically actuated valve 13102 that can control the direction of the sample either to the integrated ATR-FTIR 23110 or to the sampling site 24102 wherein a 3-axis robotic external sampling pipette 24100 can move over and extract the sample to be studied and analyzed outside the system. Another possible route will be for the sample to travel into the ATR-FTIR 23110, once that measurement is done the sample will be ejected into waste chamber 13106. An alternative route will be directly for the sample to be ejected out to the waste chamber 13106 through a 2/2 bistable pneumatically actuated valve 13102 without going into the ATR-FTIR 23110.

### Mixing Chambers

FIGS. 32A-B illustrate several isometric views of the mixing chamber and its parts, according to an example of the present disclosure. In an embodiment, the fluid consisting of cells or biological or chemical components, which we call media, is first inserted manually by the user into the mixing chambers, where gasses are bubbled and a heating element heats the fluids. The system then automatically distributes the media to either one or more biochips, the waste chamber, or any other component of the operating system. O2, CO2, and temperature sensors are used to maintain physical parameters at the levels set by the user.

In an embodiment, the pumping mechanism is pressure driven and is actuated pneumatically through air tubes multiplexed from a pressure control system.

In another embodiment, the pumping system mechanism is driven by a syringe pump where fluid is pushed through the syringe through the system.

In another embodiment, the pumping mechanism is peristaltic pump driven where the fluid in the containers is sucked and pumped into the system.

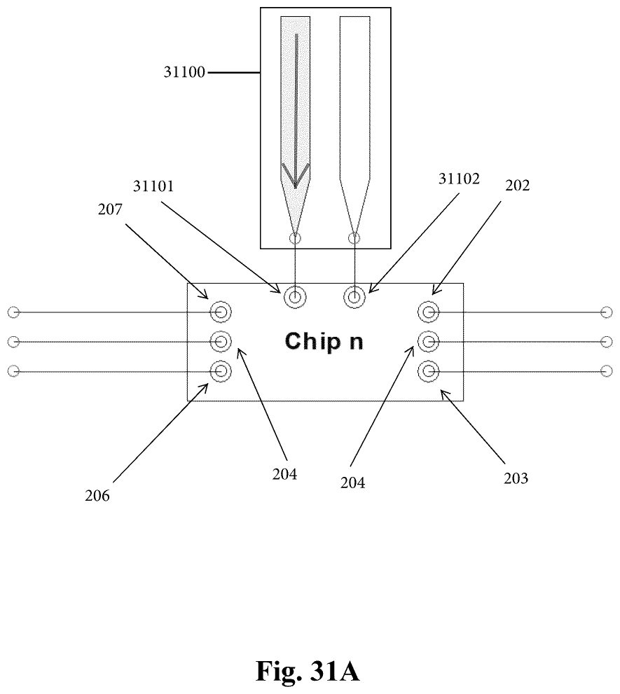

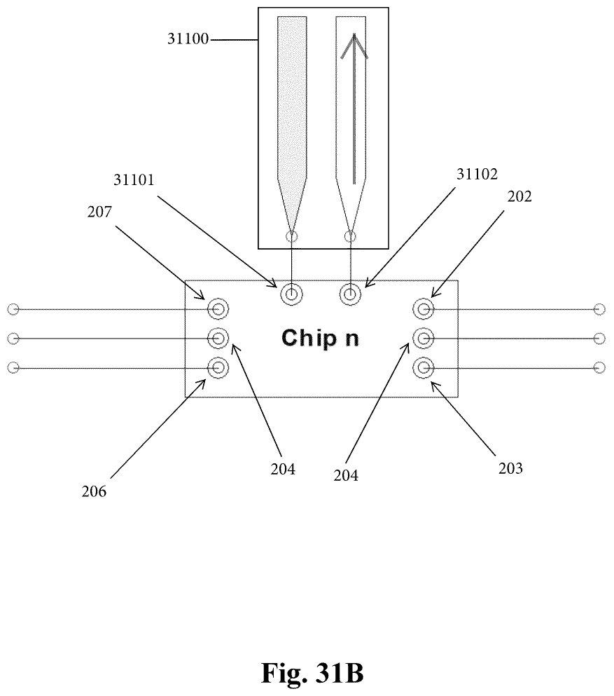

FIG. 31A and FIG. 31B show schematically the process of injecting biological gel into the biochip 100, since the biochip 100 is made up of micro-fluidic channels introducing fluid with high viscosity (like gel) might cause clogging. The biochip 100 contains multiple ports that include the first duct inlet 202 and outlet 207, second duct inlet 203 and outlet 206 where the scaffold system is implemented for the cells to attach to and form a 3-dimensional cylindrical duct. The second compartment of the biochip 100 is the stroma that's equipped with an inlet/outlet 204, since the stromal channels have a smaller inner diameter, injecting gel using the inlet and outlet might cause clogging. Hence, a dedicated stroma gel inlet 31101 and stroma gel outlet 31102 may be implemented. To prevent the gel from going through the perforated scaffold, a double-headed pipette 31100 may be used. The pipette tips used are spaced away to align with the stroma gel inlet 31101 and outlet 31102. One side of the pipette will be pumping the gel into the biochip 100 and the other will be pumping the excess gel out of the biochip 100. The gel ports 31101 and 31102 are equipped with a biocompatible self-healing membrane that prevents exposure of the gel to the outer atmosphere and causes potential contamination.

FIGS. 32A-D show the mixing chamber 13100 and its different components. The mixing chamber 13100 will house the cells and media that will be used by the human operating system 23100. The two major sections of mixing chamber 13100 are cell mixing chamber 33101 and media mixing chamber 32107. The cell mixing chamber 33101 will store the cells in their favorable conditions which will then be used by the human operating system 23100 to be seeded in the biochip 100 in the human-on-chip plate 10104. The media mixing chamber 32107 contain the media used to maintain the cultured tissues for a prolonged period of time. Both chambers are equipped with a gas source 32103 that can induce different types of gases to mimic different conditions or to maintain similar conditions as in the human body. FIG. 32D shows a closer view of the individual components of the mixing chamber 13100 wherein the cell mixing chamber 33101 contains three rows of cell chambers 33107 each row is made up of 12 individual cell chambers 33107, an assembly of 36 cell chambers 33107 will form the cell mixing chamber 33101. For the media chamber 32106, the same arrangement may be adopted where an array of 12 media chambers 32106 is interlinked into each other forming the Media mixing chamber 32107.

FIG. 32E shows the pressure/gas source assembly 32100, wherein two units, one for the media mixing chamber 32107 and one for the cell mixing chamber 33101, will act as the pressure source to supply the system with the pressure needed to pump fluids from the mixing chamber 13100 to the human-on-chip plate 10104. The pressure/gas source assembly 32100 is divided into two sections housed in the pressure/gas container 32102, the first section will be the pressure/vacuum source 32101 and the second section is the gas source 32103. Since each of the media mixing chamber 32107 and cell mixing chamber 33101 consist of 3 rows of components the pressure/vacuum source 32101 accommodates this arrangement by having 3 different pressure outputs 32105 that can be controlled individually. The gas source 32103 will be supplying the cells and media with the necessary gas mixture to maintain favorable conditions for tissue culture. Since the cells mixing chamber 33101 is made up of 36 individual cell chambers 33107 while the media mixing chamber 32107 has only 12 media chambers 32107 the gas source 32103 differs between the two, wherein the cell mixing chamber 33101 is equipped with 36 gas output 32104 while the media mixing chamber 32107 have 12 gas output 32104 that supplies the needed gas mixture.

FIG. 32F shows the individual media chamber assembly 32200 where the media chamber 13104 will store the media to be later used for referring the tissue culture. The cells require a certain mixture of gases to be continuously perfused into the media, for this reason, a gas output 32104 from the pressure/gas assembly 32100 will be connected to the media gas input 32201 on the media chamber 13104. An O2/CO2 sensor 32204 is placed underneath to monitor the dissolved gas concentration in the media, based on this reading the system will regulate the amount of gas being perfused. A media sampling port 32202 is implemented on the top cover of media chamber 13104, this port can be accessed manually by the operator to perform a regular quality test on the media. A media pressure input 32206 will be connected to the pressure/vacuum source 32101, the pressure will drive the media from the media chamber 13104 to the human-on-chip plate 10104. To do so, the media chamber 13102 is equipped with a media output port 32203 wherein the media will pass through the port to the tubing and then to the human-on-chip plate 10104.

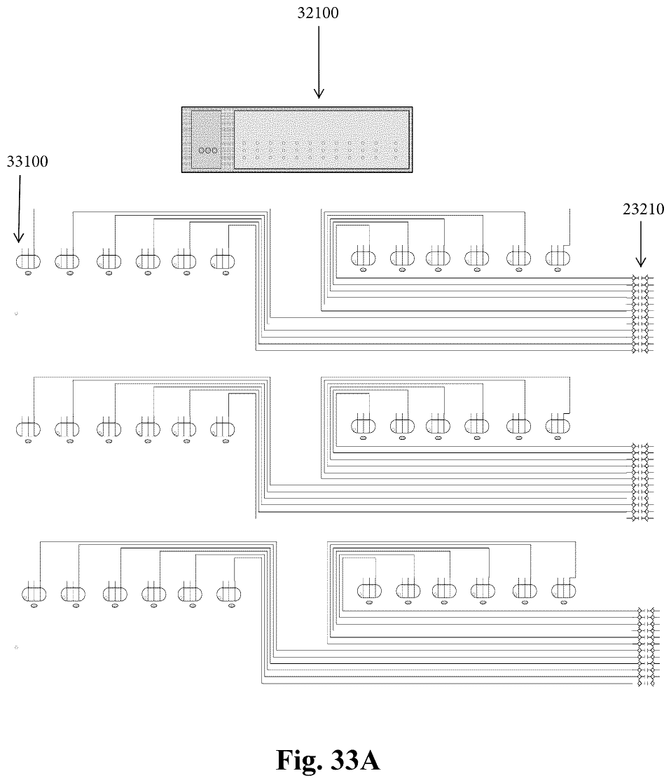

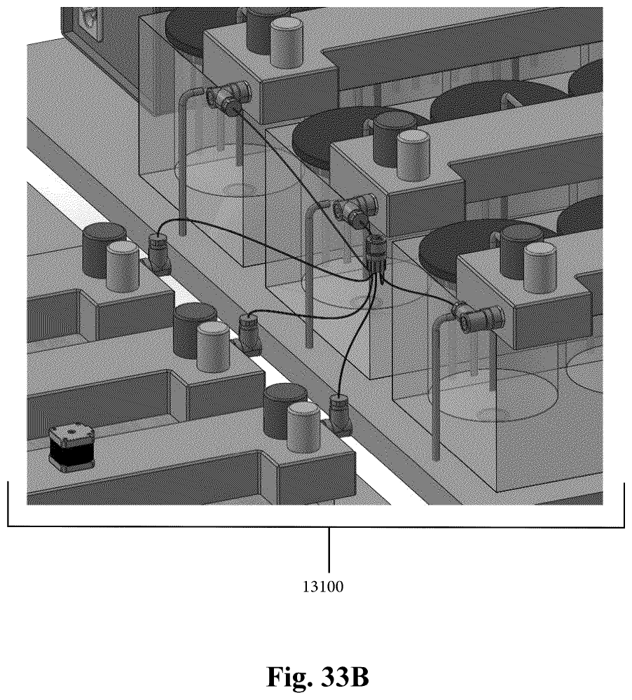

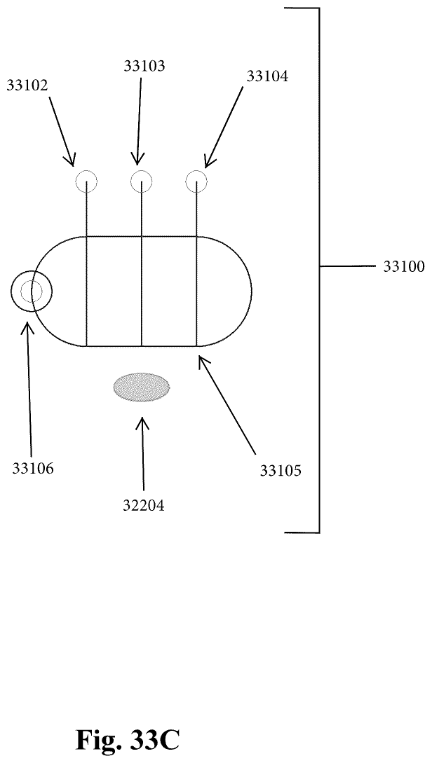

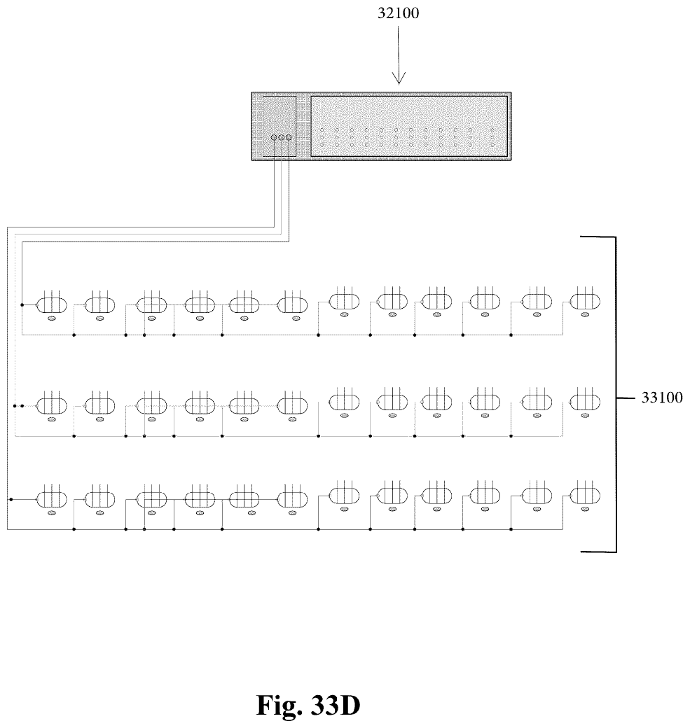

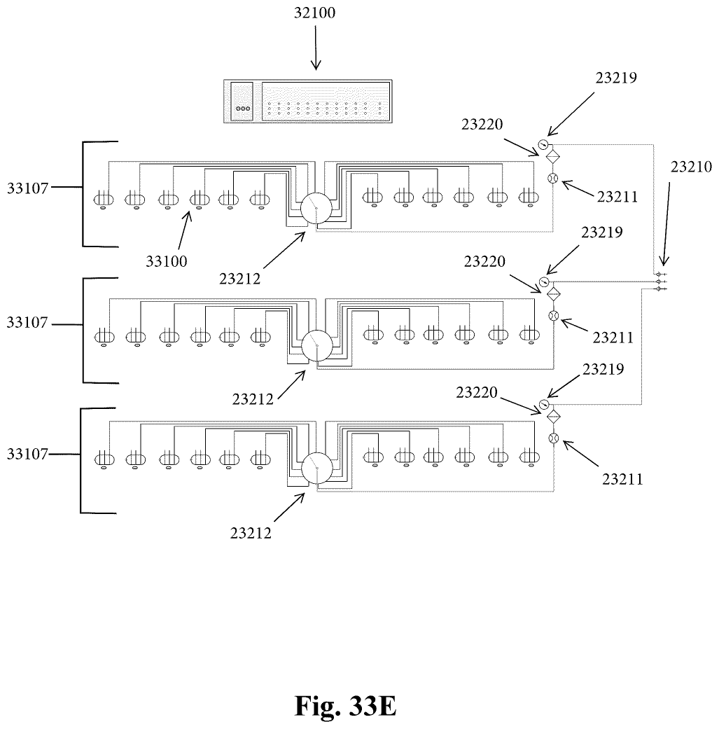

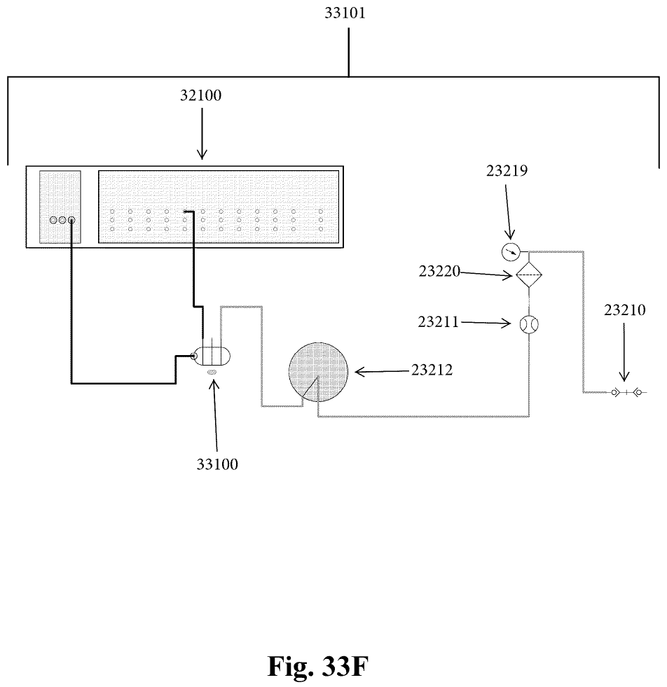

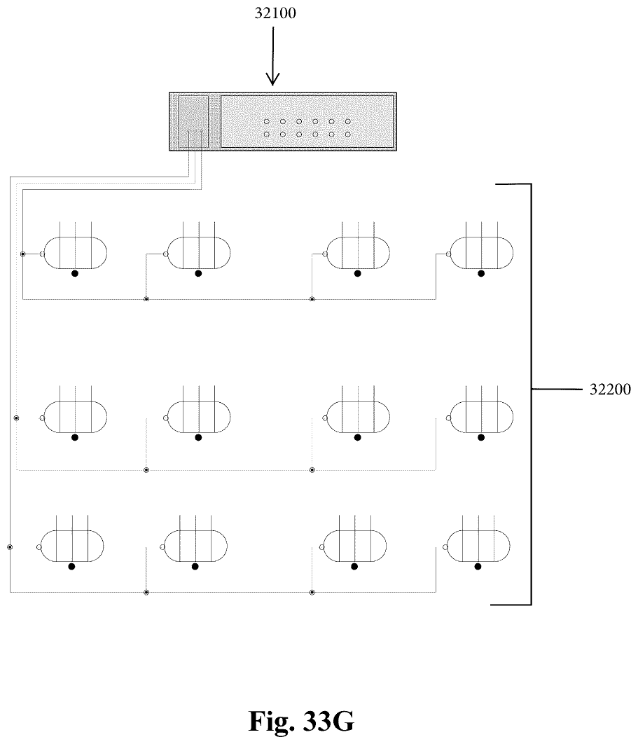

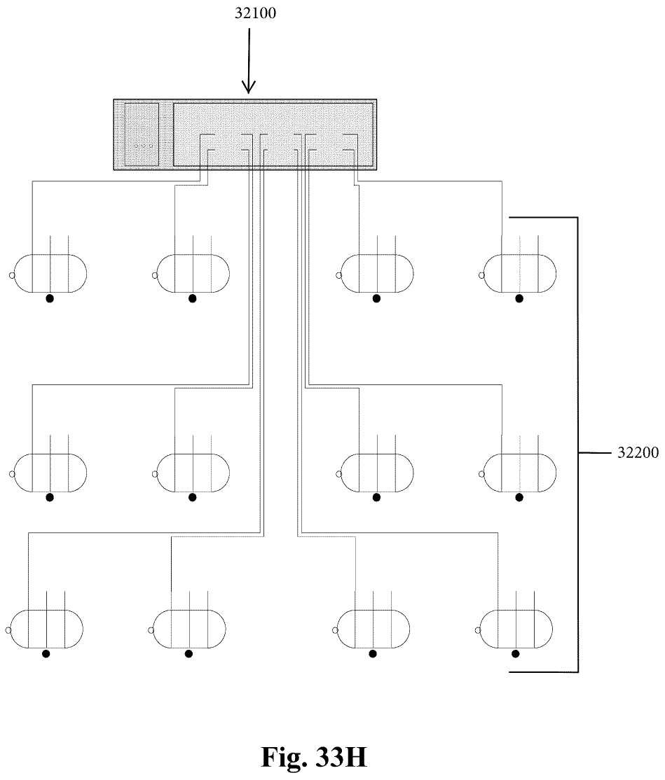

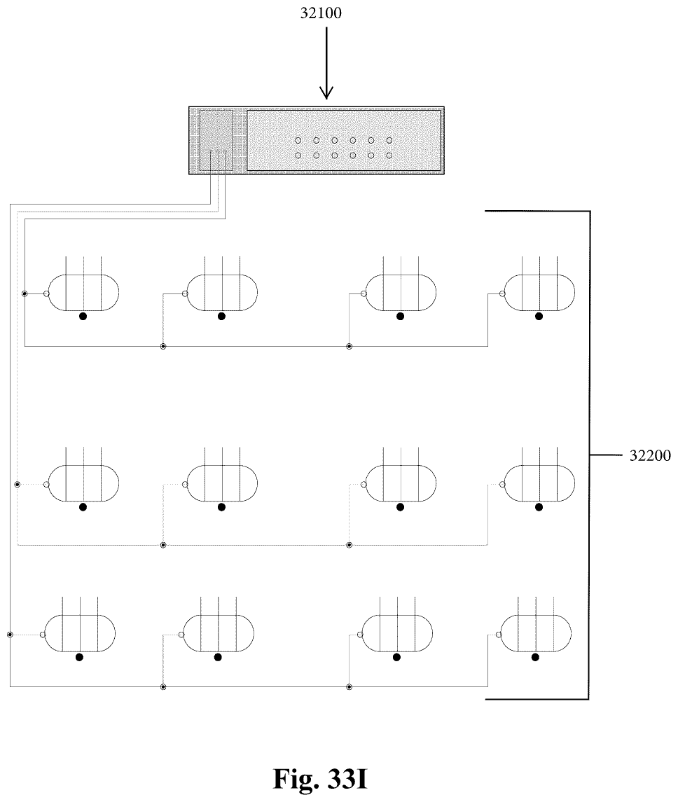

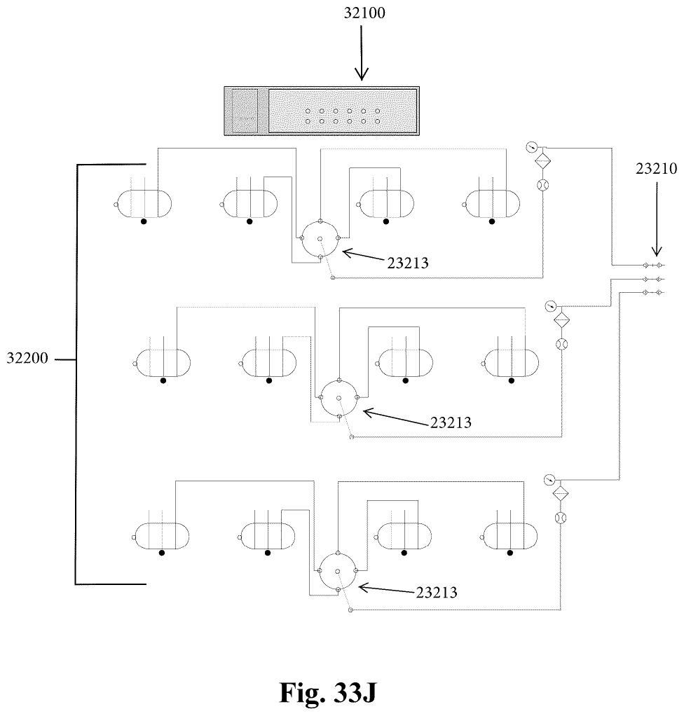

FIGS. 32G and 33J describe a schematic view of an individual media chamber 13104 connected to the media mixing chamber 32106. The assembly starts by connecting the pressure/gas source assembly 32100 to the media chamber assembly 32200. Connection lines will connect the pressure and the gas output from the pressure/gas source to the pressure and gas input in the media chamber 32200. The media will be supplied with the required gas mixture, and once it's ready the pressure will drive the fluids from the media chamber assembly 32200 through the media output port 32203 to the 4/1-way valve 23213. The 4/1-way valve 23213 rotates between 4 different positions. If the valve spool clicks in one of these positions, the 2 openings will align allowing fluid to flow from the media chamber 32200 through the valve into a flow meter 23211. A flow meter 23211 is used to monitor the fluid flow and acts as a feedback signal for the system. The flow meter is used to control the flow since the system uses pressure to drive the fluid. Using pressure to drive the fluid might cause the formation of air bubbles for this reason an air trap 23214 is placed directly after the flow meter, which will eliminate the possibility of an air bubble escaping into the fluidic operating system 23200. To further support the signals derived from the flow meter 23211 a media pressure gauge 23215 will monitor the fluid pressure, and both sensors will provide feedback signals measuring the flow rates or detecting the presence of any blockage in the tubing. The fluid will then make its way through the tubing into a quick connect 23210 that will connect the mixing chamber 13100 to the fluidic operating system 23200.

FIG. 33A shows schematically an embodiment of the cell mixing chamber 33101, wherein each individual cell chamber assembly 33100 is directly connected to a quick connector 23210 which is interim connected to the fluidic operating system 23200. The pressure/gas source assembly 32100 will pressurize the cell chamber causing the cells to flow into the human-on-chip plate 10104 passing through the fluidic operating system 23200.

FIG. 33B shows the mixing chamber 13100 consisting of the cell mixing chamber 33101 and the media mixing chamber 32107 with a focus on the quick connectors 23210 that will be used to link the mixing chamber 13100 with the fluidic operating system 23200. The quick connector facilitates the maintenance of the human operating system 23100.

FIG. 33C shows a detailed view of the cell chamber assembly 33100 where the cells will be stored to later be used in the system. The cells will need to be maintained in a stable environment, one of the major conditions is the gas mixture. The cell chamber is equipped with a gas input port 33102 that will connect to the pressure/gas source 32100, and an O2/CO2 sensor 32204 will monitor the gas concentration in the cell chamber 33105 and then relay the feedback to the operating system for any adjustments. The cell chamber is equipped with sourcing port 33103 that can be used to inject cells into cell chamber 33105 or to take a test sample. A pressure line will be connected from the pressure/vacuum source 32101 to the cell pressure input 33106 on the cell chamber 33105. The pressure will be used to pump the cells from the cell chamber to the human-on-chip plate 10104.

FIG. 33D shows the connection for the pressure source 32101 of the pressure/gas source assembly 32100 into the cell chamber pressure input 33106. The cell chamber assembly 33100 consists of 36 cell chambers 33105 that form the cell mixing chamber 33101 described in FIG. 32.

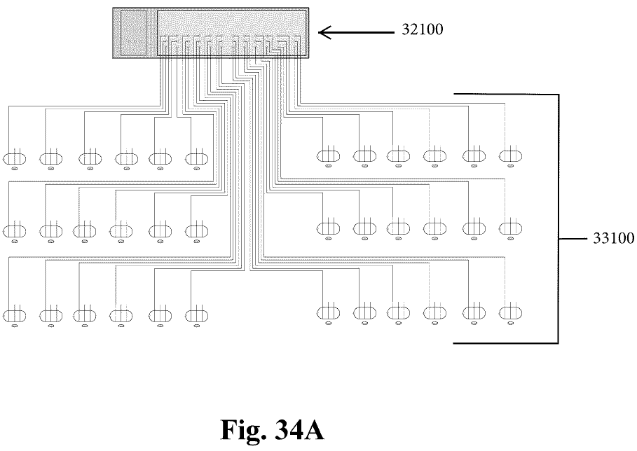

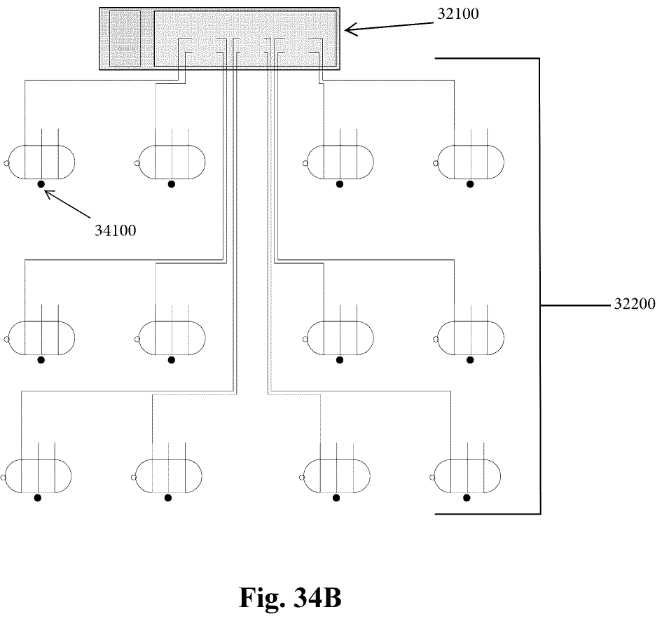

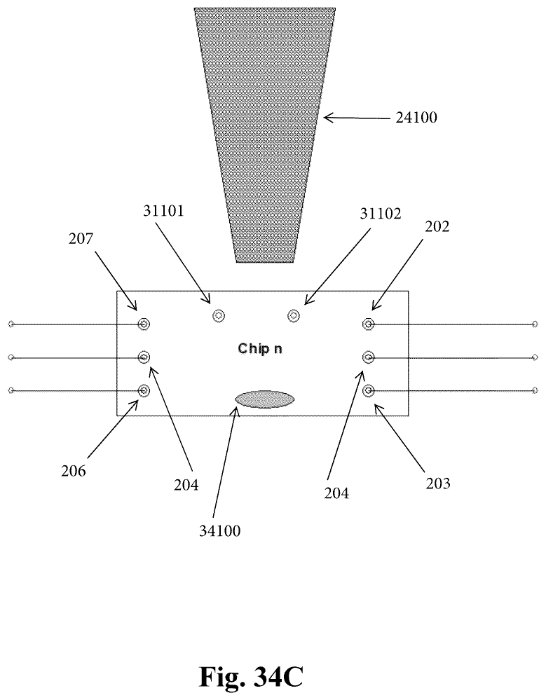

FIG. 33E-F and FIG. 34A show a schematic diagram of the cell mixing chamber 33101 explained in FIG. 32B. Each row of the cell chamber assembly 33100 forms a set of cell chambers 33107. Taking a closer look at the individual cell chamber assembly 33100 in FIG. 33F, the pressure/gas assembly 32100 is connected to the cell chamber assembly 33100, supplying it with the necessary gas mixture to maintain cell viability. The other connection point will be from the pressure output 32105 of the pressure/gas assembly 32100 to the cell pressure input 33106. The pressure will drive the cells from the cell chamber 33100 to the 12/1-way valve 23212. The 12/1-way valve 23212 rotates between 12 different positions, all the 12 cell chambers assembly 33100 are connected to this valve. Once the valve rotates in one of the 12 positions, the 2 port openings inside the valve assembly will align allowing fluids to pass through. The mixture of fluid and cells will travel through the connection tubing passing by the detection sensors. The detection sensor consists of a flow meter 23211 and a cell pressure sensor 23219. The sensors will work together to monitor the flow rate of the fluid mixture and provide feedback to the control system. This arrangement will allow the detection of any issues like clogging or blockage inside the micro-channels. As described in FIG. 33B the fluid mixture will go through the quick connector 23210 into the fluidic operating system 23200.

FIGS. 33G-H, and FIG. 34B show the connections between the pressure/gas source 32100 and the media mixing chamber 32107. As explained in FIGS. 32G and 32C, the pressure/gas source assembly 32100 supplies the media mixing chamber 32107 with the necessary pressure through the media pressure input 32206. The pressure/gas source will drive the media from the media chamber 32200 to the fluidic operating system 23200. Gases that will be perfused in the media with other nutrients will be supplied by the gas output 32104 on the pressure/gas source 32100. Controlling the amount of gas being pumped into the media will allow the operators to perform experimental tests that require a special diffused gas mixture ratio, for example when inducing hypoxia.

### Sensing Mechanism and Control for the CO2, O2 and Temperature

FIG. 34A illustrates a schematic diagram of an embodiment of the mixing chamber 13100 where all the containers have access to O2, CO2, and N2 bottles and O2 and CO2 sensors 32204, in an example of the present disclosure. While the media is in the containers of the mixing chamber 13100, gasses are bubbled and a heating element heats the fluids. O2, CO2, and temperature sensors are used to maintain physical parameters at the levels set by the user.

In an embodiment, the O2 and CO2 are pumped directly into the containers of the mixing chamber, in a closed loop control system, where optical or biochemical sensors measure the ratio of the diffused O2 and CO2 in the fluid.

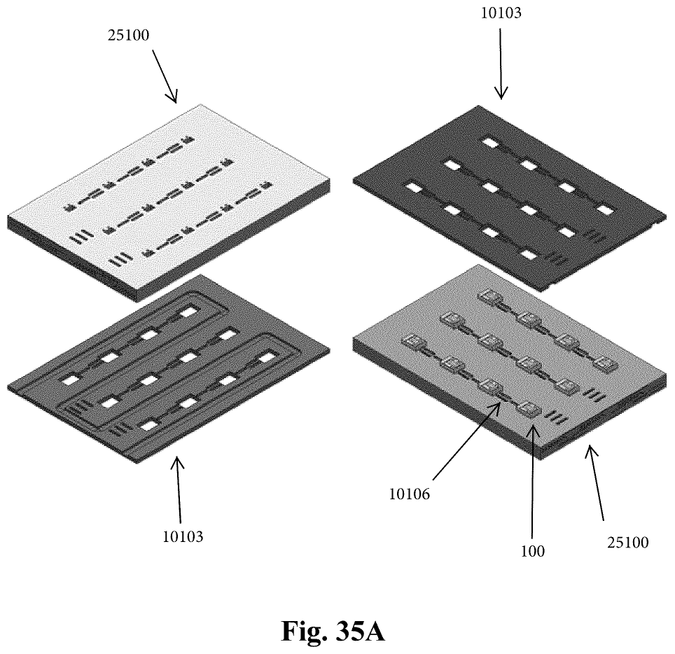

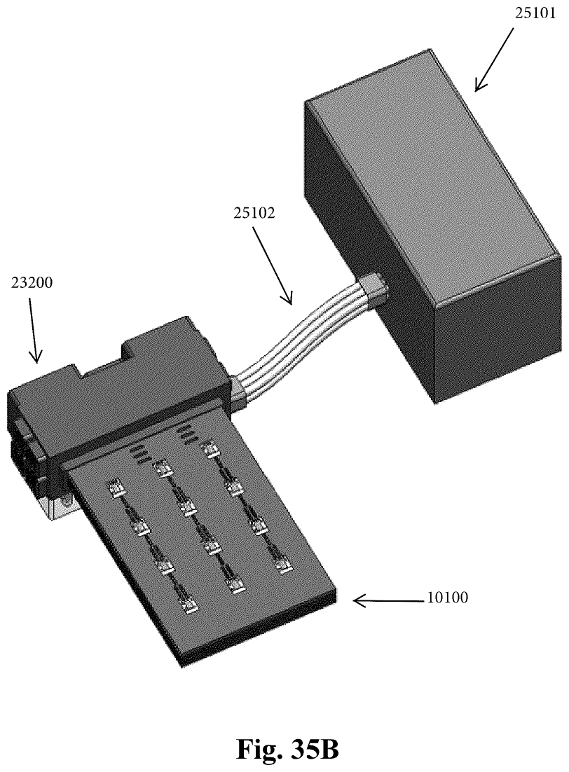

In an embodiment, the temperature is induced through a heating element and is measured using a heat sensor, where the temperature is controlled through a feedback control system as shown in FIGS. 35A and B.

FIG. 34C shows the oxygen and carbon dioxide sensors. Since a cell culture is highly sensitive to the concentration of O2/CO2, multiple sensing devices may be implemented in the system to trace back any fluctuations in the gas mixture. The first sensing level will be in the mixing chamber 13100 wherein each individual cell chamber 33105 and media chamber 13104 will be equipped with an O2/CO2 sensor that will measure and monitor the gas concentration. After that, the second sensing level will be through a chip embedded O2/CO2 sensor 34100 that will perform a continuous real-time measurement. The data collected by the chip embedded O2/CO2 sensor 34100 will provide significant information about the cell viability and functionality, especially in the case of testing tumor invasion and growth inside the biochip 100, where cancerous cells have hypoxic conditions. In some cases where the experiment requires hypoxic conditions, and such condition is needed in specific biochips the chip embedded O2/CO2 sensor 32100 will provide an exact and reliable reading in each specific biochip. To support the chip embedded O2/CO2 sensor 34100 reading a 3-axis robotic external O2/CO2 sensor 24100 is implemented. This sensor can move in 3-dimensional space over the biochips 100 found on the human-on-chip plate 10104. The 3-axis robotic external O2/CO2 sensor 24100 will work as an overall independent sensor, wherein the obtained data will be cross-referenced with the reading from the Chip embedded O2/CO2 sensor 34100 to eliminate any false measurements. The system will rely on this data to perform any modification in the gas mixture provided by the pressure/gas source 32100.

FIGS. 35A and B describe the heating and cooling process on the human-on-chip plate 10104. The heat exchange unit 25101 is connected to the fluidic operating system via tubings 25102 that will circulate heating and cooling fluid. The fluid will pass through the fluidic operating system 23200 into the human-on-chip plate 10104, one of the multiple layers of this plate is the heating plate 10103. The heating plate 10103 is machined from the inside to create pathways that the cooling and heating fluid can pass through to alter the temperature of the human-on-chip plate 10104. Since the biochips 100 are placed inside the human-on-chip plate 10104 the heat will be transferred, thus stabilizing the system at the optimal environment for the culturing tissues.

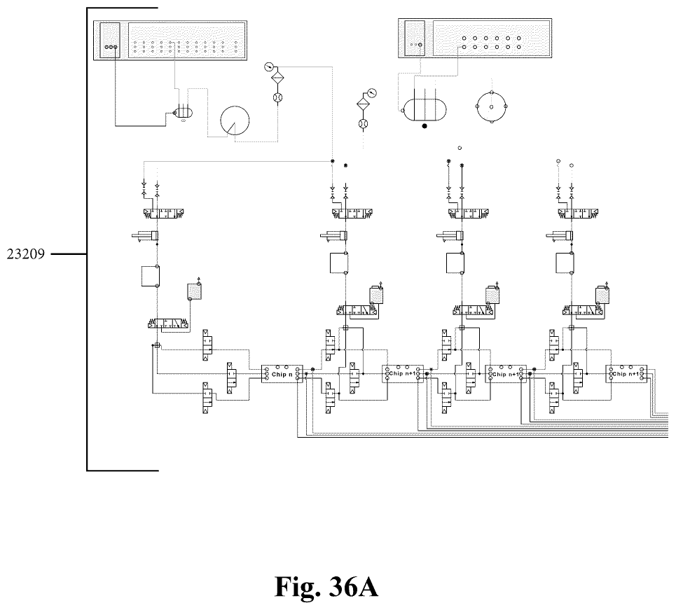

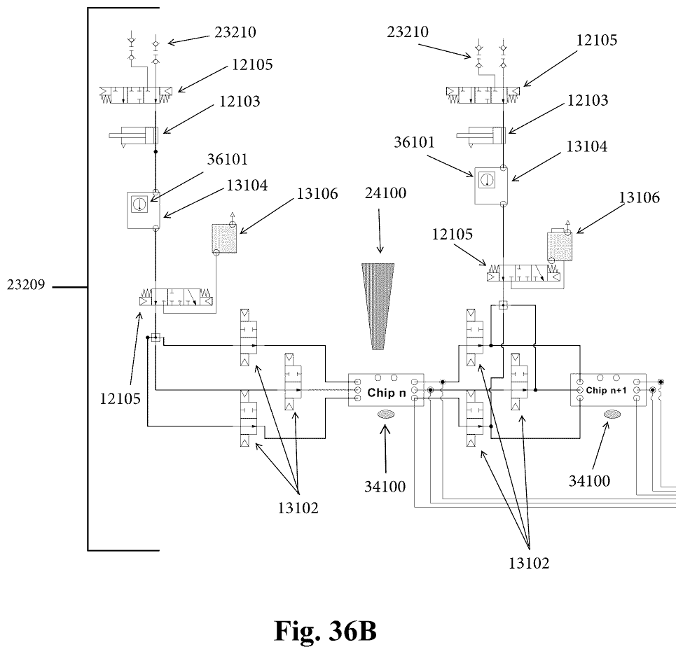

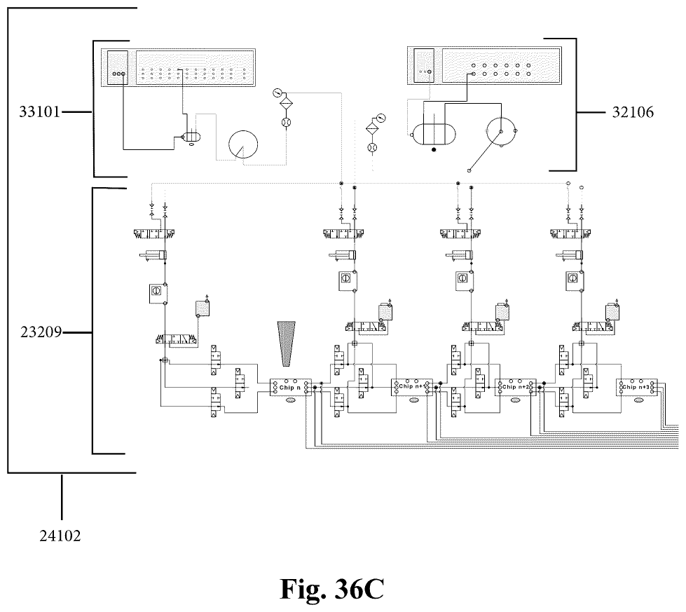

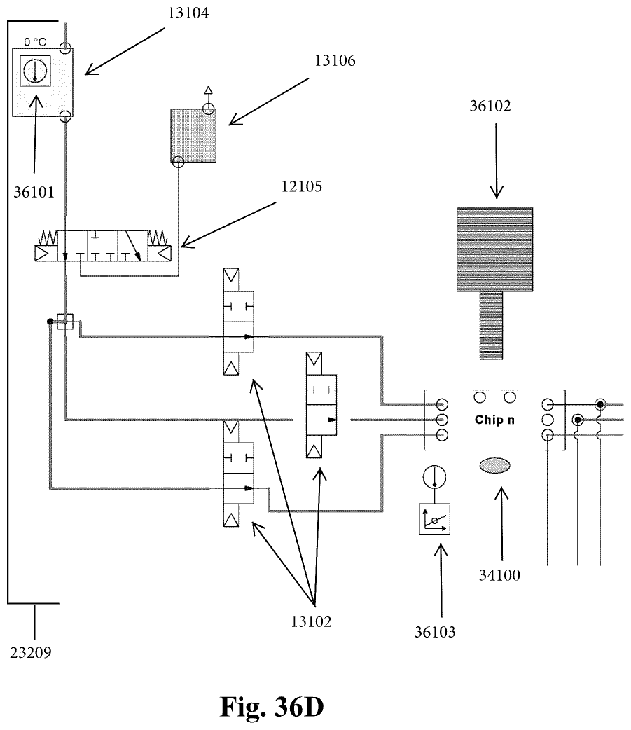

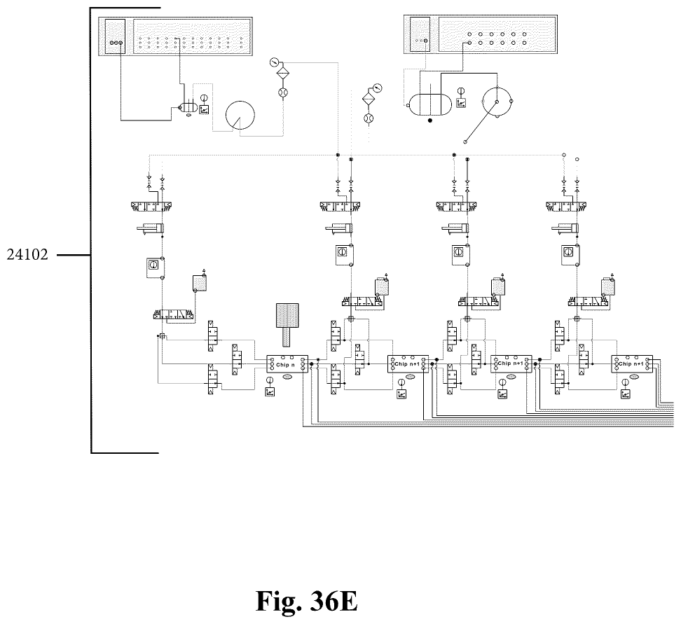

FIG. 36A shows the system initiation process, where the system will run a self-diagnosis sequence. the first stage in this sequence is to test the valves' functionality by actuating them between their open/closed position. If any valve reports an error, an operator may be notified. Once valve functionality is confirmed, the system will initiate the flushing process of all the components. This step may include washing the valves and the microchannels removing any contaminants or debris from the system and will verify that none of the valves or microchannels are blocked. The final initiation step is to circulate media in the system and the heating process will start in preparation for cell seeding. The 02/CO2 sensors described in FIG. 34C will measure the concentration of the gas mixture confirming the initiation for cell seeding.

FIGS. 36B-E show the different temperature sensors found in the system. The media and cells will be stored in the mixing chamber 13100. At this stage, the initial warm-up of the mixture will start. After the media reaches the desired temperature, it will be transferred from the media mixing chamber 32106 into the biochip 100. The media will pass by 3/3 spring-loaded pneumatically actuated valve 12105 into the media chamber 13104. The media chamber 13104 is equipped with a media chamber temperature sensor 36101 that will report back to the system forming a feedback control to maintain the desired temperature. Then the 3/3 spring-loaded pneumatically actuated valve 12105 will switch to the open position allowing the media to flow into the 2/2 bistable pneumatically actuated valve 13102, a set of three valves will control whether the flow will go into the first duct, second duct, stroma channel or into all at the same time. The chip embedded O2/CO2 sensor 34100 and 3-axis robotic external O2/CO2 sensor 24100 may measure the gas concentration as described in FIG. 34C. The same or a similar process may take place while injecting the cells from the cell mixing chamber 33101 into the human on-chip plate 10104. The final stage of temperature sensing takes place on the biochip directly. As illustrated in FIG. 36D in a human-on-chip flow diagram 23209, each biochip is equipped with an embedded temperature sensor 36103 that monitors the temperature internally. A thermal camera 36102 fixed to the 3-axis robot 21100 is used to image the human-on-chip plate 10104 for any cold or hot spots, this camera will help to assess the overall temperature of the plate.

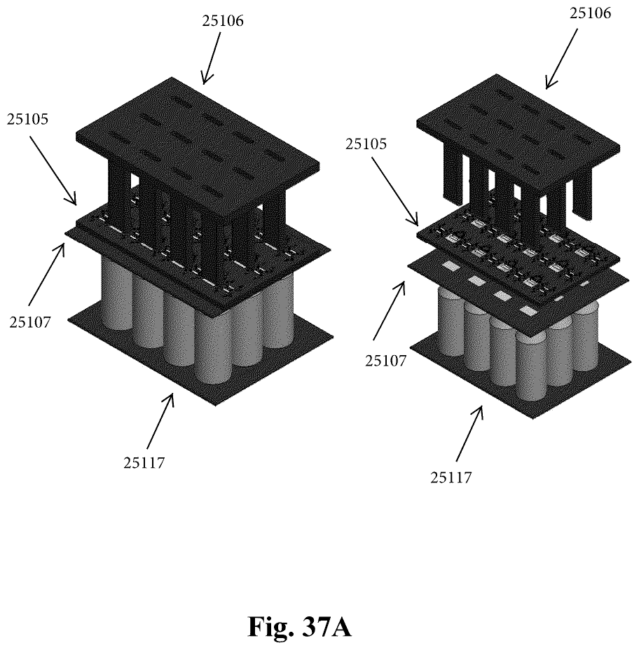

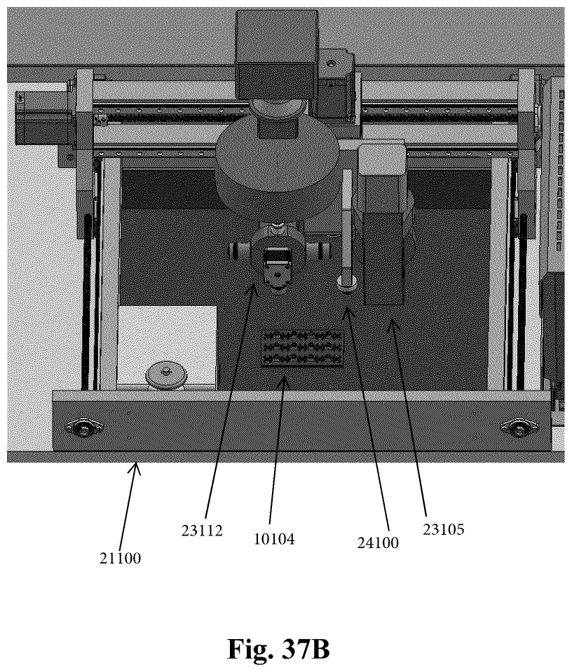

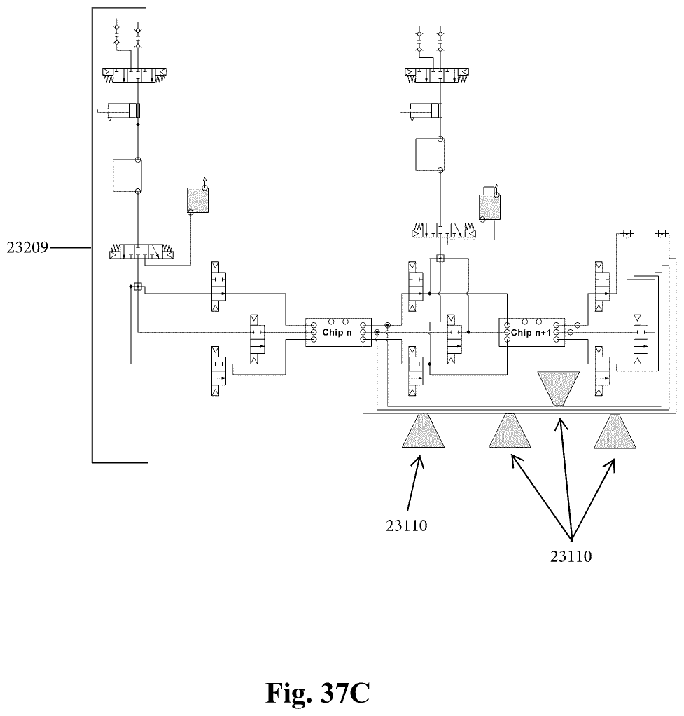

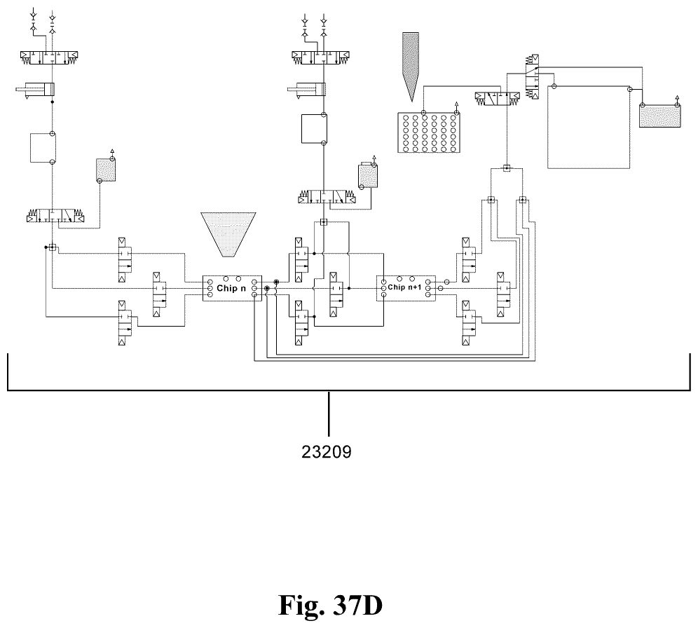

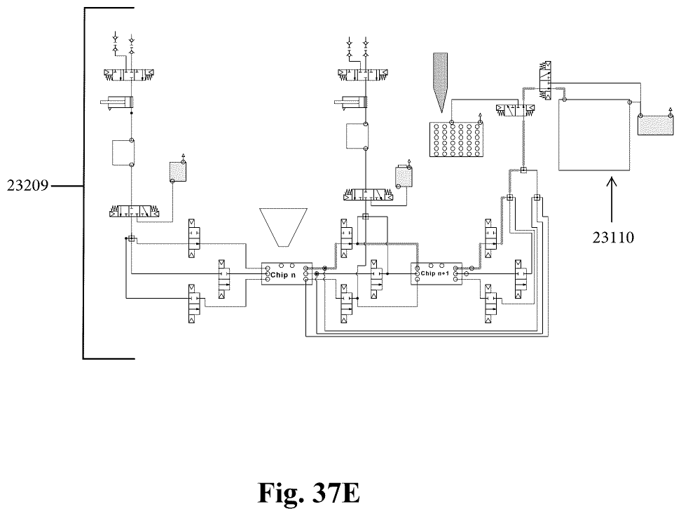

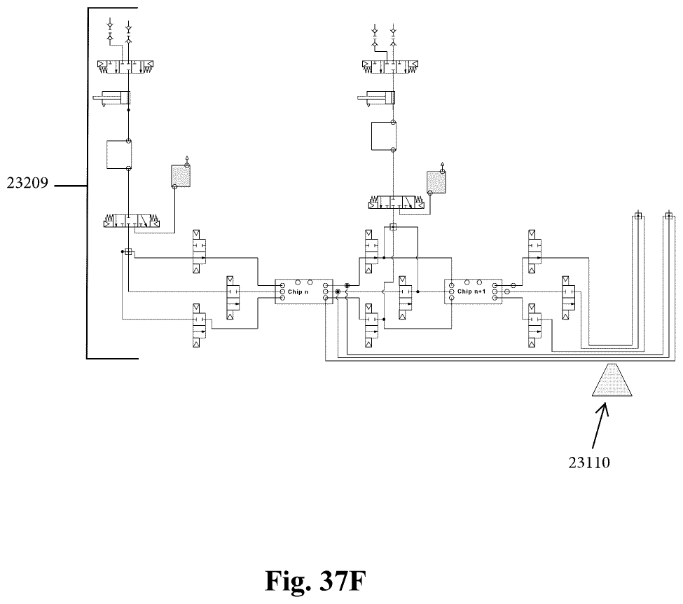

FIG. 37A shows an embodiment of the human operating system with the embedded measurement devices that consist of a spectroscope array 25106 and a microscope array 25117. The spectroscope array 25106 requires a spectroscope sensing plate 25107 that is placed underneath the human-on-chip plate 25105, where this sensing plate 25107 receives the emitting light from the spectroscope array 25106 through the biochip 100. Since the microscope array 25117, is placed underneath the spectroscope sensing plate 25107, it may disrupt the microscopic imaging. For this reason, the sensing plate can move to the side, providing a clear sight for the microscope lenses to image the tissue culture.

In another embodiment, the light source needed by the microscope can be replaced by the emitted light from the spectroscope.

FIG. 37B shows an embodiment of the human operating system where the measuring devices are fixed on a 3-axis robot. The measuring devices consist of a microscope 23112 equipped with a multi-lens turret that can rotate to switch between different magnification levels. Next to the microscope 23112 is the 3-axis robotic external O2/CO2 sensor 24100 and the spectroscope 23105. All the measuring devices can hover over the human-on-chip plate 10104 and perform analytical measurements.

### Solution Insertion

In an embodiment of the operating system 26100, in order to fill the media chamber, as illustrated in FIG. 28F-I, two steps are performed repeatedly until the media chamber is filled. In the first step, the media chamber valve is closed to block the flow to the biochip or to the waste channels. At the same time, the valve above the media chamber leading to the media source plate is open, to insert the fluid from the source plate. After opening the valves, the piston is pulled up, to pump the fluid from the source plate towards the media chamber. Since the volume of the media chamber is small (on the order of a few 100s of microliters), smaller than the volume of the tubing reaching the media chambers from the source plate, the piston will reach the top-most position before the media reaches the media chambers in the pipes. When the piston reaches its top-most position, the valve below the media chamber and the waste channel valve is opened and the valve going to the biochip channels and the top valve before the media source plate is closed. After opening and closing the respective valves, the piston is pushed down to remove the excess air from the media chamber. When the piston reaches its bottom-most position, step one may be repeated and then step two until the media fills the pipes and reaches and fills the media chamber. In an embodiment, the solution may be cell culture media.

### Solution Replacement

To empty the media chamber, as illustrated in FIG. 28C, a user of the system should perform two steps repeatedly until the media chamber is empty. In the first step, close the valve below the media chamber to block the flow to the biochip or to the waste channels, and open the valve above the media chamber before the media source plate, to push the fluid into the source plate. After opening the valves, push the piston down, to pump the fluid from the media chamber towards the source plate. Since the volume of the media chamber is small (on the order of a few 100s of microliters), the volume of the tubing reaching the media chambers from the source plate is larger, and thus the piston will reach the bottom most position before the media reaches the source plate in the piping. When the piston reaches its bottom-most position, open the valve below the media chamber and the waste channel valve and close the valve going to the biochip channels and the top valve before the media source plate. After opening and closing the respective valves, pull up the piston to fill the media chamber with additional air. When the piston reaches its top-most position, repeat step one and then step two until the media empties the pipes and reaches the source plate. In an embodiment, the solutions may be cell culture media.

### Cell Insertion

To seed cells into the biochip channels, described in FIG. 28D, the user could first insert the media containing cells into the media chamber in the same way described in FIG. 28B, and perform the method to fill the media chambers. Then the user can open the valve below the media chamber on the two sides of the waste valve to give access to the flow to the biochip, and close the valve of the waste channels to block the flow towards the waste channels. Then the user can close the valve above the media chamber before the media source plate, to block the flow from and to the source plate. After opening the valves, the user should open the outlet waste valve to push excess air or media outside the system to replace them with the cell-filled media, and then push the piston down, to pump the fluid from the media chamber towards the biochip channel. From the outlet, the user can open the channel between the different biochips if needed to seed the same cells into the different biochips so that the outlet waste channel opened would be the last biochip one needs the cells to reach. After the cells reach their intended location, the user may close all valves and stop pumping, and wait until the cells attach. After the cells precipitate on the ducts bottom surface and attach, the user may flip the full operating system, and then open the same valve and do the same steps, and then pump cells into the biochip ducts, and wait for the cells to attach now on the top surface of the ducts. After the cells precipitate on the ducts top surface and attach, the user may flip the full operating system to its initial form, and continue the experiments.

In an embodiment, cells may be inserted into the biochip by any available channel, including but not limited to the side channel. Cells may be inserted suspended in an appropriate liquid or in an appropriate polymer which will solidify.

In an embodiment, skin tissue may be cultured into the plurality of biochips to allow human-on-chip operating system to perform protocols related to, but not limited to, assessment of skin health and physiology.

In an embodiment, cells, media or any other substance may be inserted into a specific location within any channel of a biochip or any biochip within an operating system. Cells may be, also but not exclusively, sampled by delivering an appropriate enzyme or other reagent which may cause cellular detachment at specific locations. Once the cells are detached, the fluid control system will collect the suspended cells.

### Media Sampling

In an embodiment, to collect the media sample from the biochip channels, described in FIG. 28E, a user of the system could perform two steps repeatedly until the media chamber is filled with the intended sample quantity. In the first step, for both the inlet and outlet fluid channels, the user may open the valve below the media chamber on the two sides of the waste valve to give access of the flow from and to the biochip, and close the valve of the waste channels to block the flow towards the waste channels. Then the user may close the valve above the media chamber before the media source plate, to block the flow from and to the source plate. After opening and closing the intended valves, the user may push the piston controlling the inlet fluid down, and at the same rate and volume, pull up the piston controlling the outlet piston, to collect the intended sample volume in the outlet media chamber. After collecting the intended sample in the media chamber, the user may initiate step two which is transferring the collected sample up the system, by emptying the outlet media chamber. The emptying process is as described before in FIG. 28C.

In an embodiment, to collect the media sample from any of the biochip channels within the operating system, the media or any other liquid substance may be collected from the side channel.

### Cell Sampling

In one embodiment of the operating system, the same cells or tissue types can be grown across different biochips with a common duct(s). Each biochip thus represents sub-samples of the same organ. Each individual biochip can then be removed independently, at different time points, from the circulating system and taken out of the system for downstream analysis. This allows for the sampling at different time points.

In one embodiment of the operating system, the same cells or tissue types can be grown across different biochips with a common duct(s). At the time of drug dosing, the biochips can be isolated from each other. Each individual biochip can then be sampled or removed independently for downstream analysis.

To collect the cell sample from the biochip channels, described in FIG. 28F, one may perform four steps until the media chamber is filled with the intended sample quantity. In the first step, the biochips are connected together along their ducts making them one continuous tissue sample, thus each biochip taken at different time lapses, is a sample of the same tissue. Thus, in the first step this interface is needed. In the second step, the user may close the valve between the different biochip entities, to make each biochip duct a sample to be collected at a specific time-point. At this phase, the user may block the intended valves and pump, as described before, to replace the media with trypsin in the media chamber of the inlet of the biochip where the cell sample is aimed to be taken. In the third step, the user may pump trypsin into the intended media channels as described before, and collect the sample of cells into the outlet media chamber in the same manner the media sample is collected. In step four, after collecting the intended sample in the media chamber, transfer the collected sample up the system, by emptying the outlet media chamber. The emptying process is as described before in FIG. 28C.

In an embodiment, cells, media or any other substance may be sampled from a specific location within any channel of a biochip or any biochip within an operating system. Cells may be, also but not exclusively, sampled by delivering an appropriate enzyme or other reagent which may cause cellular detachment at specific locations. Once the cells are detached the fluid control system will collect the suspended cells. Sampling may occur from the inlet and outlet holes of the biochip. In some instances, the cell sampling may occur from the side channels of the operating system.

### Inserting Multiple Reagents Sequentially

In one embodiment of the operating system, reagents may be added sequentially. The media chamber is filled with the first reagent as described previously, and this is then inserted into the stromal chamber of the biochip. The media chamber is then emptied, as previously described, and filled with a different reagent which can then be inserted in the stromal chamber. This can be repeated as many times as necessary. The time between the different reagents being added to the stromal chamber is controllable and programmable. They can be added one after the other immediately or after a set interval of time.

In an embodiment, inserting reagents sequentially is essential, not only for dosing with multiple chemicals but also when running an assay including but not limited to in biochip immunohistochemistry and cell health assays.

### Tubular Architecture and Barrier Functions of Ductal Tissues

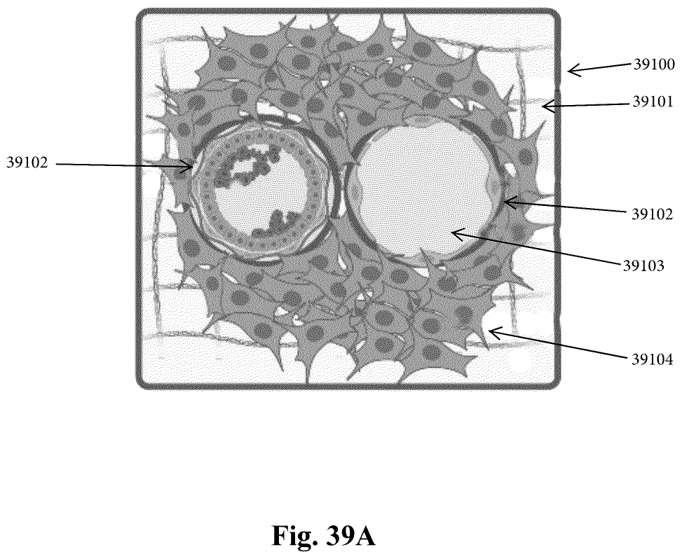

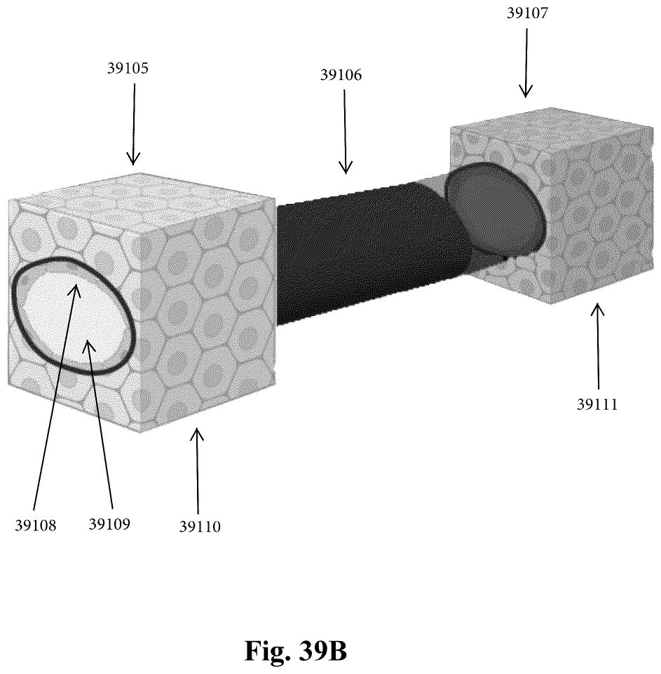

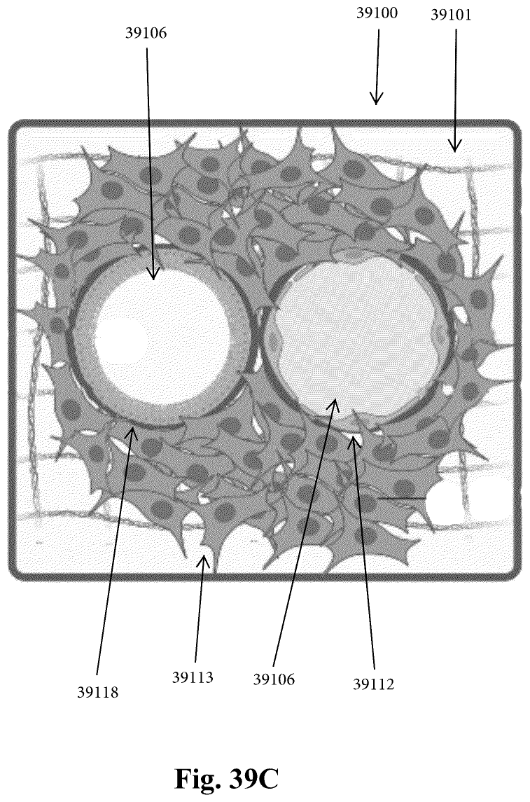

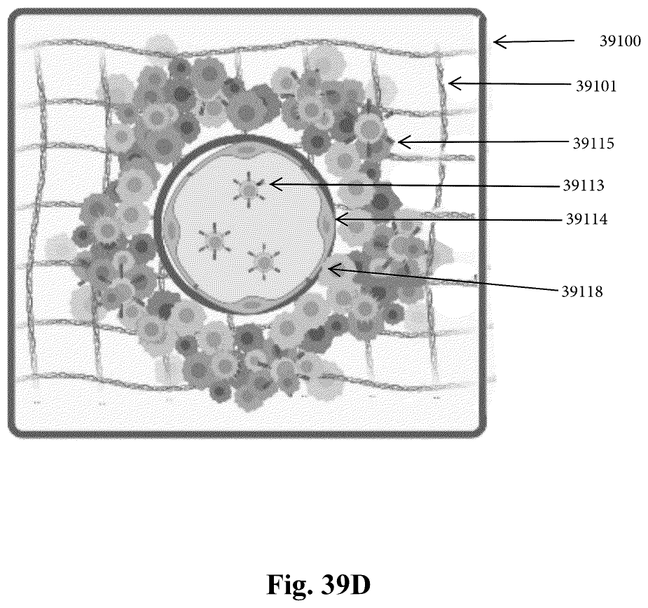

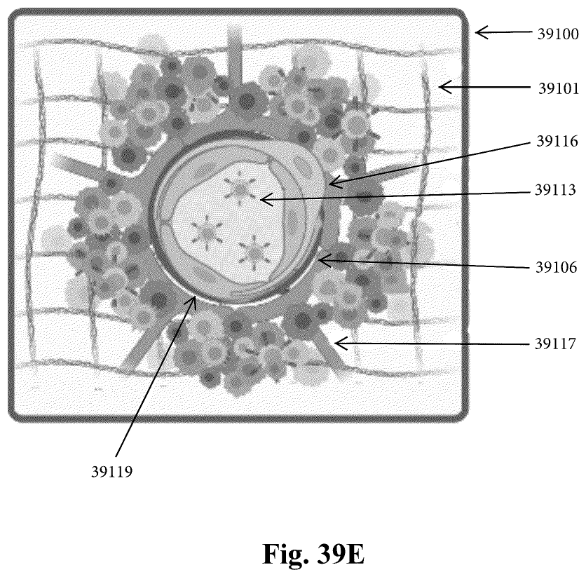

FIG. 39E shows an embodiment, where the system is used as a blood-brain barrier model for drug screening and permeation studies. Here the duct may be populated by brain microvascular endothelial central nervous system cells 39119 interfaced by tissue composed of pericytes 39116, astrocytes 39117 and neurons. This may then be used to perform a drug permeability test and/or toxicity and efficacy analysis on the neural tissue.

### Culturing of Multiple Tissue in Different Chips Connected by a Common Duct/Vessel

FIG. 39B illustrates a perspective view of the embodiment of multiple cultured tissues in biochips connected with a common duct/vessel 39106. In one embodiment of the operating system, it can be used to study the microbiota-gut-brain axis. Here, the stromal chambers 39105 and 39107 of two biochips 100, one containing intestinal epithelium cells layered with intestinal mucus and patient-derived microbiome 39110. The media in the stromal chamber 39105 is hypoxic. The duct, which connects various biochips, is seeded with vascular endothelial cells 39108 and the media flowing through it is fully oxygenated 39109. The other biochip 100 contains cultured neurons, astrocytes, and microglia 39111 embedded in a hydrogel matrix. Effects of different microbiota on the brain biochip can be assessed by microscopy or sampling of the neural cells and running ex-system protein and gene expression assays.

FIG. 39B is a perspective view of the embodiment of multiple cultured tissues in our biochips connected with a common duct/vessel 39106. In an embodiment, the system may be configured such that each biochip contains a different tissue and organ system, but these are connected in parallel and or series by common ducts or ducts which emulate the vascular and/or lymphatic systems 39109. Some biochips may have multiple ducts which may be interconnected with one or more of the other biochips 100. Taken together this would be a human-on-a-chip and is valuable to screen for off-target and/or systemic effects of drugs and the permeability of a drug through different anatomical barriers. It would also give a more accurate indication of the drug's half-life in-vivo.

FIG. 39B illustrates in an embodiment, a system that provides for at least two different tissues 39110 and 39111 to be grown in contact via a duct or ducts (vascular or lymphatic vessel or both). This also allows for tissue-tissue interaction studies.

In an embodiment, the system may be configured to simultaneously assess hepato- and nephron-toxicity in drug screenings. As both the kidney and the liver are responsible for the clearing and metabolism of xenobiotics, it is essential to assess drug efficacy in the presence of these two tissues. In this embodiment, one or more biochips contain a renal-epithelium and another (or multiple) biochips hepatic cells, as described elsewhere herein. The two tissues are connected by a duct lined with endothelial cells 39108. The drugs are dissolved within the duct, and cytotoxicity, proliferation, and apoptosis assays can be carried out on the two biochips 39105 and 39107 using spectroscopic assays. The system may also be configured to include one or more tissues in separate biochips, which are the targeted organ(s) of the tested drug.

FIG. 39A illustrates a cross-sectional view of the embodiment of a tubular architecture of tissue in a 2-duct biochip 100. In an embodiment, the system provides for at least two different tissues 39102 to be grown in direct contact, one within the duct surrounded by a second tissue 39104 within the stroma. The stroma contains another duct 39103 that contains a vascular or lymphatic duct connecting the coculture tissues to other tissues within the human-on-chip plate 10104. This allows for tissue-tissue interaction studies.

An embodiment of the system whereby a human-on-chip plate 10104 can contain different independent biochips each with a specific tissue. The plate can also simultaneously contain another set of biochips which form a human-on-chip which are interconnected by one or more ducts to other biochips.

### Liquid-Air-Interface Lung-on-a-Chip

In an embodiment, as shown in FIG. 39C, the system may be used in a model for testing gas exchange in the lungs to study lung physiology, disease, and the effect of drug candidates. The human epithelial and lung endothelial cells 39113 are cultured within the stromal gel 39101 in the external compartment 39100 containing one or two ducts 39109. One duct has human small airway epithelial cells 39118 grown attached to the inner walls of the ductal scaffold. Air is pumped through the lumen of the duct 39106. If a second duct is present, it may contain vascular cells 39112, epithelial cells, and pericytes to recapitulate capillaries. This duct has a constant flow of red blood cells at 5-0.03 cm/s and a constant pressure of 30 mmHg. Equally, in an embodiment, air could be pumped through the stromal channel 39100 of the biochip and cells can be grown in one or multiple ducts present in the biochip.

### Immune System Model

In an embodiment, the biochip 100 is capable of hosting an immune system model wherein the extracellular matrix 39101 will be injected into the biochip's stromal channel 39100. The extracellular matrix will form a biocompatible environment that will increase the stromal tissue 39115 attachment affinity. Inside the ductal channel 39106 endothelial cells will be seeded, covering the perforated 3D scaffolding system. The ductal channel will then be loaded with immune cells 39113, which will travel across the duct and might diffuse through the endothelial cells layer to the stromal channel.

In another embodiment the biochip 100 can host endothelial cells and pericytes 39114 inside the ductal channel 39106 after that immune cells 39113 are injected into the duct. Using the onboard real-time microscope imaging, the immigration of the immune cell to the stroma can be monitored and studied. The migrated immune cells may bypass the astrocytes and act on the stromal tissue forming Glioblastoma with immune cell infiltration 39116.

In an embodiment, as shown in FIG. 39D, the system provides for components of the circulating immune system, such as but not limited to T-cells, natural killer cells, and/or B-cells, to be added to individual or multiple biochips within the system. This allows for tissue (or tumor)-immune system interaction studies as well as immunotherapy efficacy studies.

In an embodiment, the system provides for engineered immune components such as, but not limited to, CAR-T cells to be added to individual or multiple biochips within the system. This allows for the system to be used in assessing the efficacy and toxicity of immunotherapies.

In one embodiment of the operating system, the immune system is modeled. This can include one or more of the following organs being mimicked on one or more biochips within the system: spleen, bone marrow, and/or lymph-nodes. These can be used either as standalone organs or connected to other organs to study immune-system interactions with other components.

### Biopsy Culture for Precision & Personalized Medicine Drug Prognosis

In an embodiment, the system may be used for precision medicine, where a biopsy or primary cells from a patient can be grown in 3D space to recapitulate the physiological/disease architecture. Drugs can be then administered to the system, according to the processes and methods disclosed herein, and specific toxicity and efficacy testing can be carried out to quantify the effectiveness of the drug or combination of drugs.

In an embodiment, primary cells from different patients can be grown on different biochips independent of each other.

In an embodiment, biochips containing different cells from the same patient can be connected to each other to form a human-on-plate.

### Microscopy Measurement Interface

In another embodiment of the operating system, the system is able to perform real-time live-cell imaging inside the biochip 100 as well as end-point imaging for the visualization of, but not limited to, cells, cell components and or tissue morphology. These can aid in the assessment of, among other things, cell proliferation, migration and/or death. The integrated system software then analyzes the images delivering quantitative data.

In one embodiment of the operating system, it performs tissue culture imaging using fluorescent and bright-field microscopy in time-lapse. In an additional embodiment of the operating system, the system is able to fix and complete all the steps to perform immunohistochemistry and/or immunofluorescence staining, subsequent automated imaging using fluorescent and/or bright-field microscopy followed by high throughput image analysis.

In another embodiment of the operating system, it can be used for testing and screening of drug and pharmaceutical ingredients.

### Spectroscopy Measurement Interface

In one embodiment of the operating system, high throughput absorbance and colorimetric assays could be performed to assess the physiological state of the tissue. Some but not all of the specific parameters which could be measured include cell health, viability, enzyme function, or cytochrome activity. In another embodiment of the operating system, high throughput fluorometric assays could be used to assess cell metabolism, viability, health, cell death, absorption, distribution, metabolism, and excretion (ADME) toxicology, or other parameters. In another embodiment of the operating system, high throughput luminescence assays could be performed to assess cell health, metabolism, viability, cell proliferation, ADME toxicology, luciferase activity, inflammation, oxidative stress, or apoptosis and/or any other assay which uses this technology.

In another embodiment of the operating system, a combination of fluorescence, colorimetry, absorbance and luminescence spectroscopy allows for single or multiplex assays which assess cell function and processes such as, but not limited to, basic indicators of cell health (cell proliferation, cell viability and cytotoxicity, the specific mechanism of cell death, such as apoptosis or necrosis) and/or cellular processes (e.g., metabolism, inflammation, cytochrome p450 activity, oxidative stress, or autophagy). In brief, in these assays a reagent is added to the biochip which interacts with a cellular component of interest and emits luminescence, fluorescence or alters the turbidity of the media which is recorded by the in-system spectrophotometer.

### Fourier Transform Infrared Spectroscopy Measurement Interface

In an embodiment of the operating system, an ATR-FTIR can be used as a measuring technique for cell expression and functionality by delivering a media sample of a minute quantity from the biochip through a set of valves and tubes that operate in a software-controlled sequence at multiple time points.

In another embodiment of the operating system, the ATR-FTIR will perform tests investigating the introduced drug efficacy or toxicity, that includes but is not limited to, element footprint, amide I, amide II, and molecular structure.

In another embodiment of the operating system, ATR-FTIR which can detect at least one sample of at least 2.3 micrometers in thickness, the ATR-FTIR system contains at least one ATR crystal with a certain reflective index. The ATR crystal might have a reflective index that ranges from 1.38 to 4.2. In an additional embodiment, the presently disclosed system may utilize the use of sample aperture signal noise reduction and/or a software base signal noise reduction by implementing mapping mode in reference to the pre-created FTIR database.

In another embodiment of the operating system, an Automated ATR cover with sensors to detect if the sample is situated properly in its place is included. An ATR crystal cleaning system consists of valves, tubes, and buffer solution controlled through software. After the cleaning is done, a test is performed by checking the reflective index and cross-referencing it with the actual index. If the reflective index is equal to the actual reflective index this means that the ATR crystal is clean. If not, another cycle of cleaning will be performed by the operating system.

ATR-FTIR measures the cell reaction to the drug by providing quantitative data on drug uptake, metabolism, and cell expression. A set of selective data from the ATR-FTIR will be stored in the operating system and then cross-referenced with the other measuring techniques to further validate the obtained results.

In FIGS. 37C-F, a third major measuring device may be an ATR-FTIR that may be integrated into the human operating system 23100 in different embodiments. In an embodiment, the human-on-chip plate 23209 schematic diagram shows each individual microchannel equipped with an ATR-FTIR 23110. This allows the system to monitor the duct channel and stroma channel individually. In another embodiment, the multiple ATR-FTIR 23110 can be replaced by one ATR-FTIR 23110 placed at the end of the system as shown in FIG. 37F in 23209.

In another embodiment, the ATR-FTIR 23110 will be placed in a separate dedicated unit inside the human operating system 23100. Tubing will route the sample of fluid to the ATR-FTIR shown in FIG. 37E.

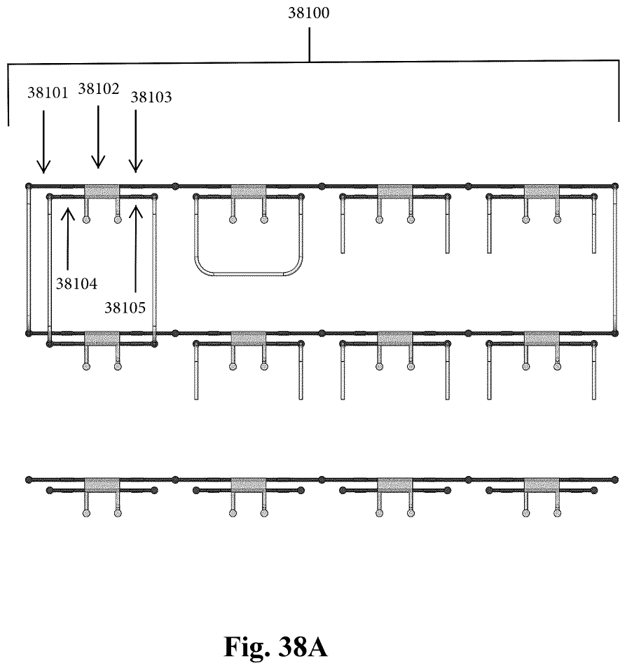

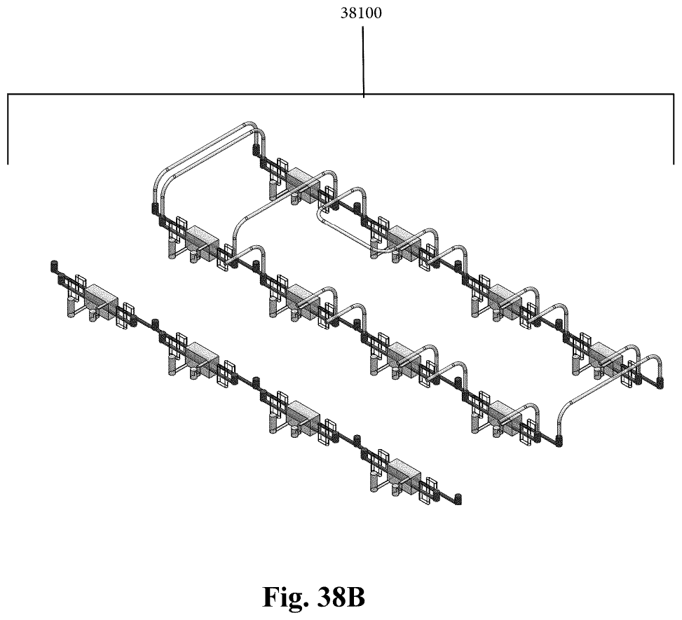

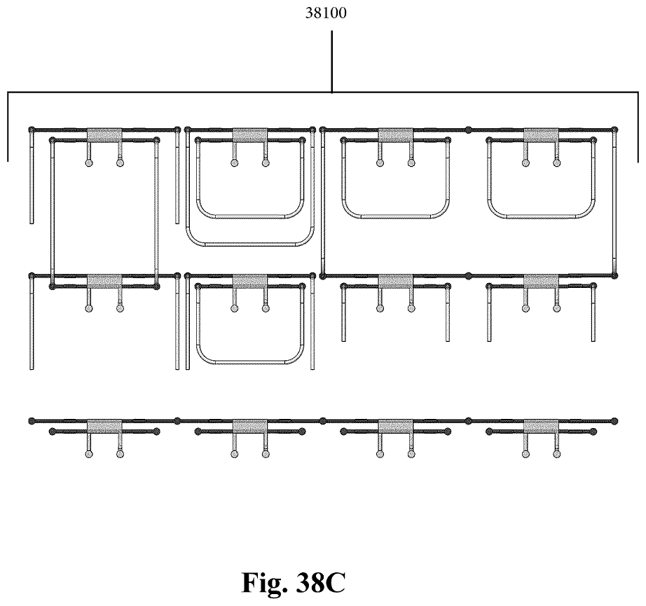

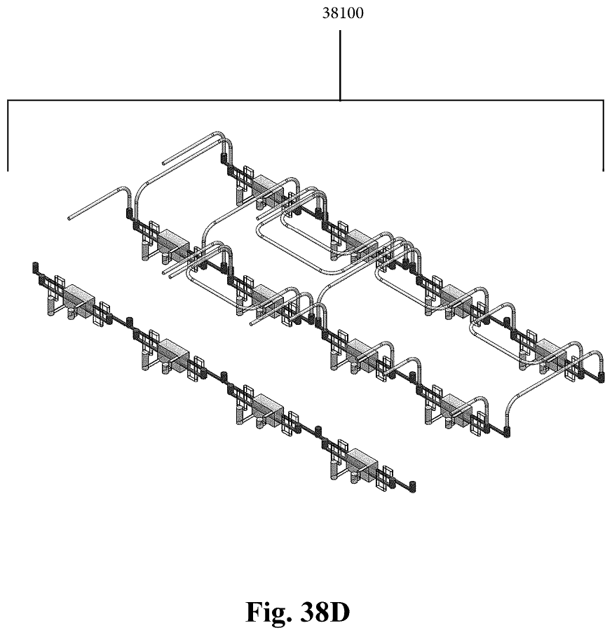

FIGS. 38A-D show the top and perspective view of the biochip interconnections 38100, according to an example of the present disclosure. In an embodiment, the biochip interconnections 38100 have a wide range of flexibility that provides the ability to freely connect biochips in the human-on-chip plate 10104. In an embodiment, multi-organs circulatory system is mimicked by connecting several biochips in a closed loop system, wherein the first biochip duct one outlet 38103 is connected to the next biochip duct one inlet 38101 and the last biochip duct one outlet 38103 is connected to the first biochip duct one inlet 38101. In another embodiment, a single organ circulatory system is mimicked by connecting duct two outlet 38105 to duct two inlet 38104 of the same biochip. In another embodiment, a biochip is connected to several biochips through the first biochip's duct one inlet 38101 and duct one outlet 38103 to mimic a multi-organs circulatory system, while also having the biochip's own single organ circulatory system through the biochip's duct two inlet 38104 and duct two outlet 38105. In another embodiment, a biochip is connected to several biochips through the first biochip's duct one inlet 38101 and duct one outlet 38103, to mimic a multi-organs circulatory system, while also having coculture circulatory system through the biochip's duct two inlet 38104 and duct two outlet 38105. In an another embodiment, multi-organs circulatory system that is connected to the media chamber outlet and the operating system is mimicked by connecting the first biochip duct one inlet to the media chamber and the duct one outlet 38103 to the next biochip duct one inlet 38101, and the last biochip duct one outlet 38103 is connected to the operating system for sampling or to the waste. In another embodiment, a single organ circulatory system is mimicked by connecting duct 2 outlet 38105 to duct two inlet 38104 of the same biochip. In this embodiment, the stroma in each biochip is controlled separately, while in another embodiment, stromas can be connected in series or parallel.

In an additional embodiment, a biological system-on-chip plate includes a plurality of ducted biochips, fluidic channeling means in fluidic communication with at least one biochip. The fluidic channeling means includes a plurality of fluidic pathways and selectable connection means providing the ability to selectively connect or disconnect fluidic communication there throughout the human-on-chip biochips. In an additional embodiment, the ducted biochips have inlet and outlet ports. In an embodiment, the ports of at least two biochips are fluidically connected to at least one fluidic pathway. In an embodiment, the fluidic channeling means enables transfer or circulation of media through ducts or stromal regions of multiple biochips. The selectable connection means enables the arrangement of biochips in series or parallel as required for a particular experiment or application. In an additional embodiment, at least one selectable connection means is configured to enable a permanent selection process to either connect or disconnect fluidic communication therethrough. In an embodiment, at least one selectable connection means includes valving means that provide reversible connection and disconnection. The valving means enable isolation of a biochip during an experiment.

In another embodiment of the biochip interconnections, a single biochip is connected in a double circulatory system, wherein the 2 circulatory systems are independent of each other. Here the first duct outlet is connected to the first duct inlet forming the first loop, then the second duct outlet is connected to the second duct inlet forming the second loop. In such an arrangement, the fluid will be in a close circulation loop.

It should be understood that various changes and modifications to the example embodiments described herein will be apparent to operating system skilled in the art. Such changes and modifications can be made without departing from the spirit and scope of the present subject matter and without diminishing its intended advantages. It is therefore intended that such changes and modifications be covered by the appended claims. Also, it should be appreciated that the features of the dependent claims may be embodied in the systems, methods, and apparatus of each of the independent claims.

Many modifications to and other embodiments of the disclosure set forth herein will come to mind to one skilled in the art to which this disclosure pertains, once having the benefit of the teachings in the foregoing descriptions and associated drawings. Therefore, it is understood that the disclosure is not limited to the specific embodiments disclosed, and that modifications and other embodiments are intended to be included within the scope of the appended claims. Although specific terms are employed herein, they are used in a generic and descriptive sense only and not for the purpose of limitation.

What is claimed:

1. A biochip operating system, the operating system comprising:
at least one biological system on chip plate;
at least one valving means;
at least one actuation means for actuating the valving means;
at least one fluidic handling means;
at least one analytical device; and
a control system for controlling the actuation means and valving means,
and the analytical device.

2. The biochip operating system of claim 1, wherein the analytical device may inspect biochips utilizing at least one of microscope images or spectral measurements.

3. The biochip operating system of claim 1, wherein the analytical device may inspect media in the fluidic handling means or fluidic pathways.

4. The biochip operating system of claim 1, wherein the operating system facilitates, semi-automates or automates at least one aspect of experimental set up including at least one of cleaning, sterilizing or preparing the system; priming the system with a biocompatible fluid; populating the at least one biological system on chip with correct cell types in correct locations as required for a desired experiment; selectably connecting the at least one biological system on chip in a correct arrangement for the desired experiment; and/or metering a correct amount of media or other compounds into the system.

5. The biochip operating system of claim 1, wherein the operating system facilitates, semi-automates or automates at least one aspect of experimental execution including at least one of circulating the media between chips as required for an experiment; controlling flow rates of fluidic movements; extracting samples, possibly at specific times or time intervals; inspecting using analytical means at specific times or time intervals and/or isolating and fixing biochips for later analysis.

6. The biochip operating system of claim 1, wherein the operating system further comprises a motion stage to move the biological system on chip plate relative to the analytical device and/or actuation means.

7. A biochip for growing multiductal tissue, the biochip comprising:
a chassis;
a plurality of internal regions;
wherein the chassis contains the plurality of the internal regions,
wherein at least one of the internal regions is in permeable or semipermeable communication with at least one other internal region; and
a plurality of fluidic media.

8. The biochip of claim 7, wherein a plurality of internal regions are tubular ducts.

9. The biochip of claim 7, wherein at least two tubular ducts are arranged with an inlet and outlet to permit through flow of a first fluidic medium.

10. The biochip of claim 7, wherein at least two tubular ducts are arranged with an inlet and outlet distinct from those of a first duct to allow through flow of a second fluidic medium that can be distinct from the first fluidic medium.

11. The biochip of claim 9, wherein at least one region is a stromal compartment external to the ducts but inside the biochip chassis.

12. The biochip of claim 7, wherein the plurality of fluidic media are the same media.

13. The biochip of claim 7, wherein the plurality of fluidic media are different media.

14. The biochip of claim 7, wherein the plurality of fluidic media comprises a combination of fluidic media types.

15. A biochip fluidic control system, the fluidic control system comprising:
at least one human-on-chip containing at least one organ-on-chip system;
wherein the at least one organ-on-chip system contains at least one ductal scaffold interfacing with at least one surrounding compartment;
at least one valving system controlling flow to each compartment;
at least one micro-pumping mechanism that can pump and control the flow to at least one compartment;
at least one actuator controlling valves on the chip and leading to the chip;
at least one actuator controlling fluid pumping to each compartment;
wherein the at least one valving system could open or close and shift flow pathways to each compartment.

16. The biochip fluidic control system of claim 15, wherein the at least one valving system may be at least one of pneumatically, mechanically, electrically, and fluidically actuated.

17. The biochip fluidic control system of claim 15, wherein the at least one valving system connects the at least one biochip to the control system.

18. The biochip fluidic control system of claim 15, wherein the at least one valving system connects the at least two biochips in a human-on-chip connection plate.

19. The biochip fluidic control system of claim 15, wherein the at least one valving system controls the at least two biochips in a human-on-chip connection plate.

20. The biochip fluidic control system of claim 15, wherein the at least one valving system controls the at least one inlet and outlet of a biochip.

21. The biochip fluidic control system of claim 15, wherein the at least one valving system connects, and controls the connection between, at least one duct to another duct.

22. The biochip fluidic control system of claim 15, wherein the at least one valving system connects at least one stroma to another stroma.

23. The biochip fluidic control system of claim 15, wherein the at least one valving system connects, and controls the controls the connection between, at least one duct to at least one stroma.

24. The biochip fluidic control system of claim 15, wherein the at least one valving system connects at least one biochip to at least one measuring device.

25. The biochip fluidic control system of claim 15, wherein the at least one valving system contains at least one bistable valve.

26. The biochip fluidic control system of claim 15, wherein the at least one valving system contains at least one open close valve.

27. The biochip fluidic control system of claim 15, wherein the at least one valving system contains at least one bistable valve and at least one open close valve.

28. The biochip fluidic control system of claim 25, wherein the at least one bistable valve maintains a state position by a beam, magnet, or any drilled geometrical shape.

29. The biochip fluidic control systems of claim 26, wherein the at least one open close valve maintains a state position by a drilled geometrical shape.

30. The biochip fluidic control systems of claim 15, wherein the at least one valving system controls media insertion in at least one biochip.

31. The biochip fluidic control systems of claim 15, wherein the at least one valving system controls cell insertion in at least one biochip.

32. The biochip fluidic control systems of claim 15, wherein the at least one valving system maintains a cell culture by media insertion.

33. A human-on-chip plate, the human-on-chip plate comprising:
at least one 3D culture biochip;
at least one micro-valve;
at least one microfluidic channel;
at least one inlet or outlet plate port;
wherein outlet ports from one chip may be connected to another chip by routing valves,
wherein multiple chips may be connected in parallel, in series or in combination and one or more chips in the human-on-chip plate may be bypassed,
wherein each fluid connection may be altered independently and at any time,
wherein the at least one micro-valve connects or separates different compartments of the biochip,
wherein at least one inlet or outlet port provides access to and blocks the at least one microfluidic channel;
wherein the at least one micro-valve may allow for sampling, changing the model flow map, and/or introducing or reducing at least one fluidic chamber,
wherein the at least one micro-valve, which may be controlled in a bistable position by actuators and sensors, is positioned at channel ports of the at least one chip,
wherein the at least one micro-valve is positioned immediately adjacent to the at least one biochip and may be controlled in a bistable position.

34. The human-on-chip plate of claim 33, further comprising at least one organ-on-chip system including at least one ductal scaffold interfacing with at least one surrounding compartment;
wherein, at least one organ-on-chip system containing at least one ductal scaffold interfacing at least one surrounding compartment.

35. The human-on-chip of claim 33, wherein the plate may be rapidly connected from a bottom or a side, and wherein fluids will be exchanged.

36. The human-on-chip plate of claim 33, wherein the human-on-chip plate is interchangeable, wherein ports on the human-on-chip will align with fitting in the biochip, and wherein the at least one micro-valve is fixed or removable.

37. The human-on-chip of claim 33, wherein the human-on-chip plate includes interconnected built-in chips.

38. The human-on-chip of claim 33, wherein the at least one micro-valve is fixed or removable.

39. The human-on-chip plate of claim 33, wherein the human-on-chip allows for fluid to be accessed from either a top or a side.

40. A multiplex logic micro-valve system, the micro-valve system comprising:
a plurality of micro-valves;
at least one fluidic pathway including at least one channel, and an inlet and outlet port;
at least one bistable mechanism;
at least one actuating mechanism;
at least one structure which encloses other elements of the micro-valve
wherein the fluidic pathway has no dead volumes,
wherein multiplexing between the plurality of valves reduces an amount of actuating inputs,
wherein the micro-valves may control fluid within each channel so that flow can be either hydrostatic or in motion.

41. The micro-valve system of claim 40, wherein at least one logic multiplex micro-valve is used to control fluid in the system.

42. The micro-valve system of claim 40, wherein the micro-valves may direct a sample to at least one sampling port.

43. The micro-valve system of claim 40, wherein the bistable mechanism is a bistable beam on a flexible tube valve.

44. The micro-valve system of claim 40, wherein the fluidic pathway may connect at least one biochip to a plurality of components within a human-on-chip system.

45. The micro-valve system of claim 40, wherein the bistable mechanism has two stable positions.

46. The micro-valve system of claim 40, wherein at least one channel contains a balloon that is pneumatically actuated.

47. The micro-valve system of claim 43, wherein the system may only require power when switching between two states, and once actuated, the bistable beam will remain in position.

48. The micro-valve system of claim 40, wherein the micro-valve is a bistable magnetic drilled-piston valve.

49. The micro-valve of claim 40, wherein the bistable mechanism contains magnetic channels.

50. The micro-valve system of claim 43, wherein the bistable beam of the bistable mechanism may be mechanically actuated and when closed, squishes a flexible tube.

51. The micro-valve system of claim 40, wherein the bistable beam of the bistable mechanism may be either solenoidly or pneumatically actuated.
